(19) United States
(12) Patent Application Publication
(10) Pub. No.: US 2024/0216584 A1
(43) Pub. Date: Jul. 4, 2024

(54) METHODS, SYSTEMS, AND DEVICES FOR GENERATING DEFINED FIBER CONSTRUCTS

(71) Applicant: Arizona Board of Regents on Behalf of The University of Arizona, Tucson, AZ (US)

(72) Inventors: Marvin Slepian, Tucson, AZ (US); Daniel Palomares, Tucson, AZ (US); Kaitlyn Ammann, Tucson, AZ (US); Syed Hossainy, Tucson, AZ (US)

(21) Appl. No.: 18/549,770

(22) PCT Filed: Mar. 10, 2022
(86) PCT No.: PCT/US2022/019714

§ 371 Date: Sep. 8, 2023

Related U.S. Application Data
(60) Provisional application No. 63/159,382, filed on Mar. 10, 2021.

(51) Int. Cl. A61L 27/54; A61L 27/18; A61L 27/38; A61L 27/56; D01D 5/00; D04H 1/435; D04H 1/56; D04H 1/728

(52) U.S. Cl. CPC A61L 27/54; A61L 27/18; A61L 27/3826; A61L 27/56; D01D 5/0038; D01D 5/0076; D01D 5/0092; D04H 1/435; D04H 1/56; D04H 1/728; D10B 2331/041; D10B 2509/00

(57) ABSTRACT

Systems, devices, and methods for generating fiber constructs via electrospinning are disclosed herein. The system for generating fiber constructs includes an ejection device and a collector. The collector contains a collection surface with patterns formed by conductive trace(s). The conductive trace(s) is arranged such that the resulting electric field lines are either undisturbed electrostatic field lines or blurred electrostatic field lines aligned around each conductive trace. Methods of generating fiber constructs using the system via electrospinning includes applying an electrostatic charge to the ejection device or a component thereof. The formed fiber construct has a grammage that is at least 5% higher than a grammage of a fiber construct formed on an un-patterned collector under the same conditions. The electrospun fiber constructs may be used for forming engineered tissues or implants, wound dressings, drug delivery formulations, and implant or device coatings.

### CROSS-REFERENCE TO RELATED APPLICATION

This application claims benefit of and priority to U.S. Provisional Application No. 63/159,382 filed Mar. 10, 2021, which is incorporated herein by reference in its entirety.

### STATEMENT REGARDING FEDERALLY SPONSORED RESEARCH OR DEVELOPMENT

This invention was made with Government Support under Grant No. T32 HL007955 funded by the National Institutes of Health. The government has certain rights in the invention.

### FIELD OF THE INVENTION

The invention is generally in the field of methods, systems, and devices for generating fiber constructs.

### BACKGROUND OF THE INVENTION

A fundamental goal of tissue engineering is repair, augmentation or frank replacement of organs or organ components due to compromise occurring as a result of disease or injury (Roy, et al., BioMed Research International 2020, 2020, 1-23; Sreekala, et al., Materials Today: Proceedings 2020). A vital component of tissue engineering is fabrication of functional scaffolds, or stroma, upon which organ-specific cells may populate, function and thrive (Tang, et al., Tissue Engineering Part B: Reviews 2020; Honig, et al., Journal of Functional Biomaterials 2020, 11, 47; Lowen and Leach, Advanced Functional Materials 2020). Native tissue stroma are typically complex three-dimensional porous structures with a wide range of interstices, pore sizes, complex layered arrangements and other irregular topographies. The spectrum of stromal configurations relates to differing needs and optima for specific cell types and organ function. Attempting to recapitulate these structures in vitro or in vivo has been difficult to date. A variety of approaches have been utilized to attempt this including: growing cell monolayers with subsequent cell-based remodeling of the underlying matrix; degrading fabricated matrices to create pores; decellularizing/devitalizing organs ex vivo to generate residual basal matrices for repurposed use; cell aggregation techniques with admixed matrix elements; and a range of layered matrix fabrication techniques (Fitzpatrick and McDevitt, Biomaterials Science 2015, 3, 12-24; Rena, et al., Journal of Tissue Engineering and Regenerative Medicine 2015, 11, 942-965; Chung and Park, Tissue Engineering Part A 2009, 15, 1391-1400; D'Amore, et al., Biomaterials 2016, 107, 1-14). However, each of these methodologies has defined, and often significant limitations (Sreekala, et al., Materials Today: Proceedings 2020; Leong, et al., Biomaterials 2003, 24, 2363-2378; Rajab, et al., Artificial Organs 2020, 44, 1031-1043).

Electrospinning utilizes an applied electrical field to induce an electrical potential between an extruded polymer solution and a grounded collector (Huang, et al., Composites Science and Technology 2003, 63, 2223-2253; Teo, et al., Science and Technology of Advanced Materials 2011, 12, 013002; Teo and Ramakrishna, Nanotechnology 2006, 17, R89-R106; Zhang and Chang, Advanced Materials 2007, 19, 3664-3667). In a typical electrospinning system, the grounded collector is an anchored, flat conductive metal surface, generating a solid fibrous sheet of electrospun material (Huang, et al., Composites Science and Technology 2003, 63, 2223-2253; Teo, et al., Science and Technology of Advanced Materials 2011, 12, 013002). More complex approaches including rotating cylinders, liquid collectors that spindle fibers, and related methodologies have been examined in efforts to control fiber properties and configuration (Huang, et al., Composites Science and Technology 2003, 63, 2223-2253; Teo, et al., Biotechnolgy Journal 2006, 1, 918-929; Wu and Hong, Bioactive Materials 2016, 1, 56-64; Baji, et al., Composites Science and Technology 2010, 70, 703-718; Nemati, et al., NanoConvergence 2019, 6).

In practice, electromagnetic force creates a fine jet of polymer solution, allowing solvent to rapidly evaporate during extrusion, leaving behind polymeric fibers on the electrically grounded collector (Huang, et al., Composites Science and Technology 2003, 63, 2223-2253; Teo, et al., Science and Technology of Advanced Materials 2011, 12, 013002).

Previous work has focused on modulating both the material and collector properties to further improve the biocompatibility of electrospun substrates (Balguid, et al., Tissue Engineering Part A 2009, 15, 437-444; Wong, et al., Polymer 2008, 49, 4713-4722; Teo, et al., Biotechnology Journal 2006, 1, 918-929).

Nevertheless, the use of electrospinning to produce the variety of different scaffolds needed in tissue engineering remains limited by the random nature of the geometry and topography of constructs typically generated by electrospinning systems and methods.

There remains a need for improved systems and methods for the fabrication of electrospun fiber constructs.

Therefore, it is the object of the present invention to provide systems for the fabrication of electrospun fiber constructs.

It is a further object of the present invention to provide methods of using an electrospinning system to form electrospun fiber constructs.

It is a further object of the present invention to provide methods of using the electrospun fiber constructs.

It is a further object of the present invention to provide devices incorporating the electrospun fiber constructs, such as for use in tissue engineering, delivery of therapeutic and/or diagnostic agents.

### SUMMARY OF THE INVENTION

Systems and methods for generating fiber constructs via electrospinning are disclosed herein. Also disclosed are uses for the fiber constructs and devices and compositions incorporating the fiber constructs. The systems for generating fiber constructs includes an ejection device and a collector. Typically, the ejection device includes a reservoir and an ejector. The collector includes a collection surface configured to receive the polymer that is ejected by the ejection device and arrange it to form fiber constructs having a selected pattern. The collection surface contains a conductive trace or a plurality of conductive traces and a non-conductive region or a plurality of non-conductive regions located thereon. The conductive trace or the plurality of conductive traces form(s) a pattern on the collection surface. Typically, the collector contains a conductive trace area from about 5% to about 99% of the total of the conductive trace area and a non-conductive area.

The collection surface can have any desired pattern. For example, the pattern can include regularly spaced and/or irregularly spaced traces. Examples of patterns on the collection surface are lines, checkerboard, squares, spirals, helices, zigzags, zigzags, sinusoidal curves, non-sinusoidal wave forms, and circles, and a combination thereof.

When in use, an electrostatic charge is applied to the ejection device or a component thereof, which produces electrostatic field lines between the ejection device and the collector. Typically, the conductive trace(s) in the pattern is arranged such that the resulting electric field lines are undisturbed electrostatic field lines and/or blurred electrostatic field lines aligned around the conductive trace or each conductive trace in the plurality of conductive traces.

Optionally, the system for generating fiber constructs via electrospinning also includes a power supply, a pressurization component, a temperature control component, a position control component, one or more sensors, a controller, or an output device, or a combination thereof.

Methods of generating fiber constructs via electrospinning using the systems described herein are also disclosed. Generally, the method includes applying an electrostatic charge to the ejection device or a component thereof. A polymer solution is contained within the reservoir of the ejection device. The polymer solution contains one or more biocompatible polymers and a solvent, and optionally one or more active agent(s) and/or one or more additive(s). Examples of suitable polymers for use in the polymer solution are natural polymers, synthetic biodegradable polymers, or synthetic non-biodegradable polymers, or a combination thereof.

Following application of the electrostatic charge, the polymer solution in the reservoir of the ejection device is extruded out of the ejection device via the ejector. The ejector is configured to direct the polymer solution towards the collection surface of the collector. During ejection, the polymer solution is further directed to form patterned fiber constructs via the electrostatic field lines between the ejector and the collection surface. Following ejection, the polymer solution and forms the fiber construct thereon. Typically, the formed fiber construct has a grammage that is at least 5% higher than a grammage of a fiber construct formed on an un-patterned collector under the same conditions.

Optionally, the method includes selecting a collector containing a pattern on the collection surface that is configured to form fiber construct having a grammage that is at least 5% higher than a grammage of a fiber construct formed on an un-patterned collector under the same conditions. This selection step occurs prior to the step of applying an electrostatic charge to the ejection device or a component thereof.

The resulting electrospun fiber constructs contain polymeric fibers formed from the polymer solution. The electrospun fiber construct can have a grammage in a range from about 18 μg/mm² to about 100 μg/mm². The electrospun fiber construct can include an open area, for example the open area can be from about 8% to about 99% of the total of the open area and a fiber area.

The polymeric fibers of the electrospun fiber construct can have the same or similar diameter; for example, the diameters can be in a range from about 10 nm to about 10 μm. Optionally, the polymeric fibers in a construct have two or more different diameters, where each diameter is in a range from about 10 nm to about 10 μm.

The electrospun fiber construct can be used on its own or may be combined with active agents, excipients, and/or carriers to form compositions. The electrospun fiber constructs may be used for forming engineered tissues or implants, wound dressings, drug delivery formulations, and implant or device coatings. In these embodiments, the electrospun fiber constructs may include active agents, excipients, and/or carriers suitable for the particular use.

### BRIEF DESCRIPTION OF THE DRAWINGS

FIG. 1 is a schematic of an exemplary electrospinning system. The exemplary system includes a flow-controlled syringe pump, a polymer (PLGA) solution, a high voltage supply, and a grounded collector. The grounded collectors were acid-etched to create differing fiber designs.

FIGS. 2A-2D are images of exemplary target designs and resultant electrospun PLGA fibers. FIG. 2A shows five conductive collector designs etched on printed circuit board (scale bars represent 1 mm). FIG. 2B shows PLGA fiber patterns electrospun onto corresponding collector designs (scale bars represent 1 mm). FIG. 2C shows low-powered 50×SEM images of PLGA patterned fibers (scale bars represent 50 μm). FIG. 2D shows high-powered 5000× SEM images of PLGA fiber constructs on the conductive portion of the collector (scale bars represent 4 μm).

FIGS. 3A-3B are graphs showing stress versus strain of PLGA fibers and films. FIG. 3A shows stress versus strain curves of PLGA fibers. FIG. 3B shows stress versus strain curve of spin-coated PLGA film. Samples were strained until failure. Each curve represents data collected from a single run.

FIG. 4 is a bar graph showing contact angle of electrospun PLGA scaffolds. Contact angle ranged from 98 (Lines) to 134 (Solid). Increased contact angle corresponds to greater hydrophobicity. Values are mean±standard error (n=9). * indicates p<0.05; ** indicates p<0.01; **** indicates p<0.001.

FIG. 5A is a bar graph showing cell adhesion and retention on PLGA scaffolds after 24 hours. Vascular smooth muscle cells adherent post washing were counted per 10× field utilizing fluorescence imaging after 24 hours. Values represent mean cell count±standard error (n=10, measurements were taken from 2 different fiber constructs, at 5 different areas for each fiber construct); * indicates p<0.05 in reference to spin coat sample; # indicates p<0.05 in reference to solid PLGA fiber scaffold. FIGS. 5B-5E are graphs showing corresponding fiber scaffold properties. FIG. 5B shows cell count (per high power field 10×) as a function of fiber target coverage. FIG. 5C shows cell count (per high power field 10×) as a function of residual open area. FIG. 5D shows cell count (per high power field 10×) as a function of normalized fiber excess. FIG. 5E shows cell count (per high power field 10×) as a function of fiber grammage. Values represent mean cell count.

FIGS. 6A-6D are images of exemplary target designs. FIG. 6A is an image of an exemplary lined pattern. FIG. 6B is an exemplary image of sinusoidal pattern. FIG. 6C is an image of an exemplary checkerboard pattern. FIG. 6D is an image of an exemplary zigzag pattern.

FIGS. 7A-7D are diagrammatical illustrations of electrostatic field lines aligned around conductive trace(s). FIG. 7A shows undisturbed electrostatic field lines aligned around a conductive trace in the form of a line. FIG. 7B shows undisturbed electrostatic field lines aligned around conductive traces arranged in three parallel lines. FIG. 7C shows blurred electrostatic field lines aligned around conductive traces in the form of six parallel lines. FIG. 7D shows blurred electrostatic field lines aligned around conductive traces arranged as five disorganized lines.

FIGS. 8A-8M are schematic illustrations of exemplary target designs. FIG. 8A is an illustration of an exemplary anti-parallel double helix pattern. FIG. 8B is an illustration of an exemplary double helix pattern. FIG. 8C is an illustration of an exemplary asymmetrical zigzag pattern. FIG. 8D is an illustration of an exemplary spiral pattern. FIG. 8E is an illustration of an exemplary overlapping circles pattern. FIG. 8F is an illustration of an exemplary double helix pattern. FIG. 8G is an illustration of an exemplary anti-parallel double helix pattern. FIG. 8H is an illustration of an exemplary asymmetrical zigzag pattern.

FIG. 8I is an illustration of an exemplary anti-parallel double helix pattern. FIG. 8J is an illustration of an exemplary overlapped sinusoidal pattern. FIG. 8K is an illustration of an exemplary intersecting wavy lines pattern. FIG. 8L is an illustration of an exemplary double spiral pattern. FIG. 8M is an illustration of an exemplary sinusoidal pattern.

FIG. 9 is a schematic showing the angle (θ) between a horizontal plane which intersects with the ejector and is perpendicular to the collection surface and an imaginary longitudinal line that intersects with the horizontal plane and runs through the center of the tip of the ejector.

### DETAILED DESCRIPTION OF THE INVENTION

### I. Electrospinning Systems

Electrospinning systems for generating defined fiber constructs are described herein. Generally, the electrospinning system includes an ejection device and a collector.

The collector contains a conductive pattern on a surface that is contacted by the ejected polymer (“collection surface”). Collection surfaces can have preformed conductive patterns to achieve particular electrostatic field distributions. In these cases, a collection surface that has the desired conductive patterns to achieve particular electrostatic field distribution is selected. Optionally, a conductive pattern is formed on a collection surface to achieve a desired electrostatic field distribution.

When in use, the collection surface is configured to receive the polymer that is ejected by the ejection device and arrange it to form fiber constructs having a selected pattern.

### A. Components of Electrospinning System

### 1. Ejection Device

Typically, the ejection device includes a reservoir and an ejector attached thereto.

a. Reservoir

When in use, the reservoir holds a spinning dope, such as a polymer solution or any other materials capable of being electrospun, such as a polymer melt. Typically, the reservoir is a container that may be sealed in a pressure-tight manner, such as a syringe. The reservoir can have two longitudinal ends, where one end is sealed and the other end is attached to a connecting portion of an ejector.

b. Ejector

The ejector is attached to a longitudinal end of the reservoir via a connecting portion of the ejector, such that when in use, the spinning dope is extruded and/or drawn by an electrostatic force through the ejector. Exemplary ejectors include, but are not limited to, needles and nozzles.

The ejector can have a single connecting portion via which a reservoir is attached to or two or more connecting portions where each connecting portion is attached to one reservoir. For example, a co-axial nozzle contains two connecting portions where a first connecting portion is attached to a first reservoir and a second connecting portion is attached to a second reservoir.

i. Diameter of Needle or Nozzle

The ejector has a tip portion through which the spinning dope is extruded. Typically, the tip portion of the ejector has an inner diameter sufficient for the spinning dope to extrude from the reservoir and form a jet towards the collector. For example, when the ejector is a needle or nozzle, the needle or the top of the nozzle can have an inner diameter in a range from about 0.08 mm to about 4.5 mm, from about 0.1 mm to about 4.0 mm, from about 0.1 mm to about 3.5 mm, from about 0.1 mm to about 3.0 mm, from about 0.1 mm to about 2.5 mm, from about 0.1 mm to about 2.0 mm, from about 0.1 mm to about 1.5 mm, from about 0.1 mm to about 1.0 mm, from about 0.08 mm to about 2.5 mm, from about 0.08 mm to about 2.0 mm, from about 0.08 mm to about 1.5 mm, from about 0.08 mm to about 1.0 mm, from about 0.08 mm to about 0.5 mm, or from about 0.1 mm to about 0.5 mm, such about 0.35 mm. Optionally, the needle or nozzle is a standard needle or nozzle from 34 gauge to 6 gauge (the larger the number the smaller the diameter). For example, the needle or nozzle is a standard needle or nozzle from 34 gauge to 6 gauge, from 34 gauge to 8 gauge, from 30 gauge to 6 gauge, from 30 gauge to 8 gauge, from 28 gauge to 6 gauge, from 28 gauge to 8 gauge, from 34 gauge to 10 gauge, from 30 gauge to 10 gauge, from 28 gauge to 10 gauge, from 28 gauge to 12 gauge, from 28 gauge to 15 gauge, from 28 gauge to 18 gauge, or from 25 gauge to 20 gauge, such as 23 gauge. Use of the term “about” describes values either above or below the stated value in a range of +/−10%.

ii. More than One Reservoir and/or Ejector

Optionally, the ejection device can include more than one reservoir and/or more than one ejector. For example, the ejection device includes two or more reservoirs, where each reservoir is configured to contain a polymer solution that is the same as or different from the polymer solution in the other reservoir(s). The two reservoirs are attached to a single nozzle, such as a coo-axial nozzle, where one reservoir is attached to a first connecting portion of the nozzle and the other reservoir is attached to a second connecting portion of the nozzle. The nozzle may include more than one tip portion, where the inner diameter of at least one tip portion is the same as or different from the inner diameter of the other tip portions.

Optionally, the ejection device includes two or more reservoirs, where a first reservoir is configured to contain a polymer solution and a second reservoir is configured to contain an active agent solution or suspension. The reservoirs are attached to a single nozzle, such as a co-axial nozzle, where the first reservoir is attached to a first connecting portion of the nozzle and the second reservoir is attached to a second connecting portion of the nozzle. The nozzle may include more than one tip portion, where the inner diameter of at least one tip portion is the same as or different from the inner diameter of the other tip portions.

Optionally, the ejection device includes more than one reservoir and more than one ejector, where each reservoir is attached to one ejector. For example, the ejection device includes two reservoirs and two needles of the same inner diameter or different inner diameters. In some embodiments, each reservoir is configured to contain a polymer solution that is the same as or different from the other reservoir. In some embodiments, a first reservoir is configured to contain a polymer solution and a second reservoir is configured to contain an active agent solution or suspension.

Each reservoir is attached to one needle. For example, the ejection device includes an array of reservoirs and two or more nozzles. Each reservoir holds a polymer solution that is the same as or different from the other reservoirs. At least two reservoirs are connected to one co-axial nozzle and each of the reservoirs is connected to its own nozzle. Each nozzle may include more than one tip portion, where the inner diameter of at least one tip portion is the same as or different from the other tip portions. For example, the ejection device includes an array of reservoirs and two or more nozzles. At least one reservoir holds a polymer solution and at least one reservoir holds an active agent solution or suspension. At least two reservoirs are connected to one co-axial nozzle and each of the reservoirs is connected to its own nozzle. Each nozzle may include more than one tip portion, where the inner diameter of at least one tip portion is the same as or different from the other tip portions.

### 2. Collectors

Generally, the electrospinning system includes a collector or more than one collector. When in use, the collector is grounded. The collector contains a collection surface that is contacted by the ejected polymer. The collection surface can have any shape; for example the surface can have the shape of a rectangular, square, circle, polygon, or triangle.

The collection surface contains a conductive trace or a plurality of conductive traces, and a non-conductive region or a plurality of non-conductive regions. Each trace in a plurality of traces is discontinuous with the other traces on the conductive surface. Each trace can be in the form of a continuous straight line, a continuous curved line, a continuous wavy linear, a combination of connected straight or curved line segments. A single trace is a continuous, or uninterrupted conductive material. For example, a trace can be in the shape of a line, a spiral, a helix, a zigzag, a sinusoidal curve, non-sinusoidal wave forms (e.g. saw tooth wave), or a circle. The term “non-conductive region” refers to a non-conductive portion of the contact surface that is defined by conductive traces. Each non-conductive region in a plurality of non-conductive regions on a conductive surface is discontinuous with the other non-conductive regions, i.e. each non-conductive region is separated from the other non-conductive regions on all of its sides by one or more conductive traces.

The conductive trace or the plurality of conductive traces form(s) a pattern on the collection surface. The collection surface can have any desired pattern. For example the pattern can include regularly spaced and/or irregularly spaced traces.

a. Conductive Traces

The collection surface of the collector contains a conductive trace or a plurality of conductive traces. The conduction trace or the plurality of conductive traces forms a conductive pattern on the collection surface.

i. Materials Forming Traces

The conductive trace is formed by a conductive material. Conductive materials suitable for forming the conductive traces include, but are not limited to, gold, chromium, platinum, iron, nickel, copper, silver, aluminum, p-dot, stainless steel, mercury, tungsten, a metal alloy of metal described herein, carbon-based materials (such as carbon powder, carbon nanotubes, etc.), semi-conductors (such as silicon and germanium), and oxides/sulfides of the metal described herein.

For example, the conductive trace is formed by a metal, such as gold, chromium, platinum, iron, nickel, copper, silver, tungsten, or stainless steel.

For example, the conductive trace is formed by copper.

ii. Dimension of Traces

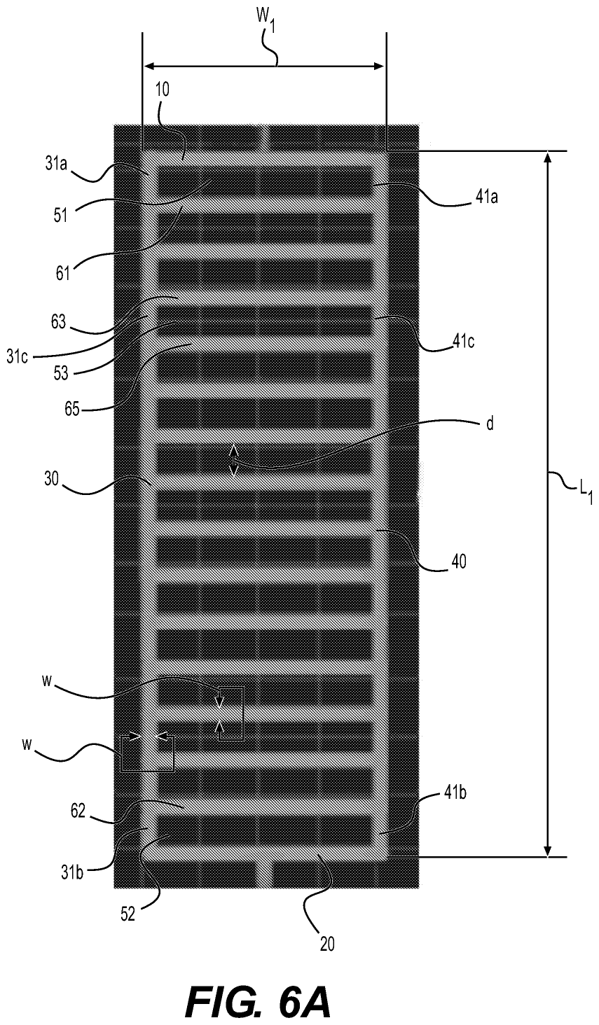

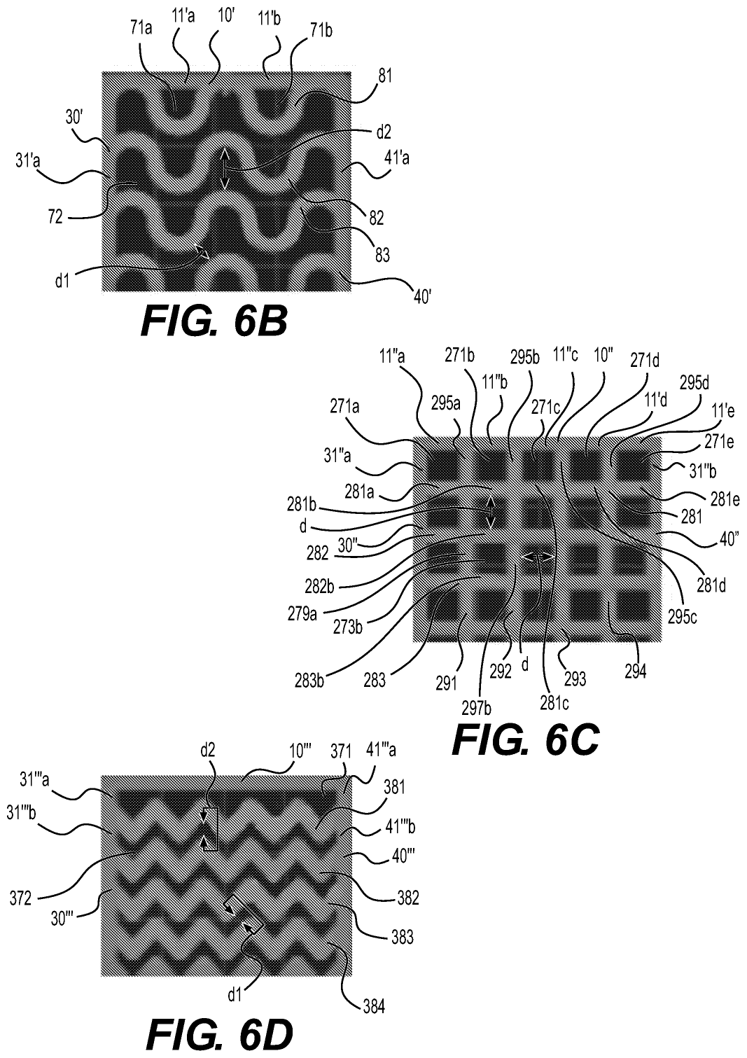

The conductive trace has a length and a width, which together is referred to as the dimensions of the conductive trace. For example, as shown in FIG. 6A, conductive traces of two different dimensions form a pattern. A first group of conductive traces, such as 30 and 40, has a length (L₁) of 30.00 mm and a width (w) of 0.635 mm; a second group of conductive traces, such as 10 and 20, has a length (W₁) of 10.00 mm and a width (w) of 0.635 mm. The values shown for the length and width of each conductive trace in FIG. 6A are exemplary. These dimensions can be the same or different, or modified as needed, to form a desired construct on a particular scale. However, each of these dimensions can be independently varied, as needed, to form a desired construct on a particular scale. The length and width of each conductive trace define an area for each conductive trace, i.e. L₁×w. When the collection surface of the collector contains two or more conductive traces, each conductive trace can have a length or width that is the same as, similar to or different from the other conductive traces. The dimensions of the conductive trace can be varied based on the shape and size of the collection surface depending on the scale of the collection surface. The dimensions of the conductive trace can be varied to influence the distribution of electrical field lines between the ejection device and the collector and thus influence the degree of overlap among the electrical field lines. The dimensions of the conductive trace together with the pattern formed by the conductive trace or plurality of conductive traces as described below, can be selected to produce a fiber construct having particular parameters, e.g. fiber deposition density (i.e. grammage) and open area of the construct described below.

Optionally, the conductive trace can have a width in a range from about 10 μm to about 10 mm, from about 20 μm to about 10 mm, from about 50 μm to about 10 mm, from about 0.1 mm to about 10 mm, from about 10 μm to about 5 mm, from about 10 μm to about 1 mm, from about 50 μm to about 5 mm, from about 50 μm to about 1 mm, from about 0.1 mm to about 5 mm, or from about 0.1 mm to about 2.5 mm, such as about 0.6 mm.

The total length of a conductive trace formed from a plurality of connected line segments is that sum of the lengths of each line segment. The length of a trace containing one or more curved line segments is the sum of the arc length for each curve.

iii. Patterns

On the collection surface, the conductive trace or the plurality of conductive traces forms a conductive pattern on the collection surface of the collector. The collection surface can have any desired pattern. For example, the pattern can include regularly spaced and/or irregularly spaced traces.

When in use, an electrostatic charge is applied to the ejection device or a component thereof, which produces electrostatic field lines between the ejection device and the collector. Typically, the conductive pattern is selected to achieve a desired electrostatic field distribution between the ejector and the grounded collector. The terms “electrostatic field” and “electric field” are used interchangeably herein.

Collection surfaces can have preformed conductive patterns to achieve particular electrostatic field distributions. Optionally, the collection surface is initially un-patterned or does not contain the complete desired pattern and a conductive pattern is formed on a collection surface to achieve a desired electrostatic field distribution.

The conductive pattern is designed such that when an electrostatic charge is applied to the ejection device or a component thereof, the resulting electrostatic field lines produced between the ejector and the collector are either undisturbed lines or blurred lines aligned around each conductive trace (see, e.g., FIGS. 7A-7D). Optionally, when an electrostatic charge is applied to the ejection device or a component thereof, the resulting electrostatic field lines aligned around each conductive trace are undisturbed, i.e. distinct from each other (see, e.g., FIGS. 7A and 7B). For example, the resulting electrostatic field lines aligned around each conductive trace do not overlap with each other. Optionally, when an electrostatic charge is applied to the ejection device or a component thereof, the resulting electrostatic field lines or a portion of the group of electrostatic field lines aligned around each conductive trace overlap with each other. The overlap of electrostatic field lines results in cancelation or other modulation of the electrical field, producing blurred or otherwise distorted electrostatic field lines. For example, when electrostatic field lines aligned around each conductive trace or a portion of the electrostatic field lines aligned around each conductive trace completely or partially overlap with each other, the resulting electrostatic field lines are blurred (see, e.g., FIGS. 7C and 7D).

Optionally, when an electrostatic charge is applied to the ejection device or a component thereof, the resulting electrostatic field lines or a portion of the group of electrostatic field lines between the ejector and the grounded collector are distorted and show fringe effects near the edge of the pattern on the collection surface (see, e.g., FIG. 7D).

Exemplary patterns formed by the conductive trace or the plurality of conductive traces include, but are not limited to, lines, checkerboard, squares, spirals, helices, zigzags, zigzags, sinusoidal curves, non-sinusoidal wave forms, and circles, and a combination thereof. For example, the pattern formed by a single conductive trace can be a spiral, a helix, a circle, zigzags, sinusoidal curves, non-sinusoidal wave forms, such as sawtooth waves, or a combination thereof. For example, the pattern formed by a plurality of conductive traces can be a combination of lines, checkerboard, squares, spirals, helices, zigzags, zigzags, sinusoidal curves, non-sinusoidal wave forms, and/or circles.

Optionally, the collection surface contains a plurality of conductive traces, where two or more conductive traces (“boundary conductive traces”) define the boundaries of a target on the collection surface. The term “target” refers to 2-D shape defined by boundary conductive traces. The target has an area which is the total of the conductive trace area and non-conductive area enclosed in the boundary conductive traces. For example, as shown in FIG. 6A, the collection surface contains boundary conductive traces 10, 20, 30, and 40 that define the boundaries of a target. A plurality of parallel straight line conductive traces is located within the boundary conductive traces. The term “non-conductive area” refers to the total surface areas of non-conductive regions within the target.

The total area of the target can be in a range from about 100 μm² to about 100 cm², from about 200 μm² to about 100 cm², from about 300 μm² to about 100 cm², from about 400 μm² to about 100 cm², from about 500 μm² to about 100 cm², from about 100 μm² to about 50 cm², from about 100 μm² to about 10 cm², from about 200 μm² to about 50 cm², from about 500 μm² to about 50 cm², from about 500 μm² to about 20 cm², from about 500 μm² to about 10 cm², from about 1 cm² to about 50 cm², from about 1 cm² to about 20 cm², or from about 1 cm² to about 10 cm², such as about 3 cm², about 4 cm², or about 5 cm².

The pattern on the collection surface can be selected to produce a fiber construct having particular parameters, e.g. fiber deposition density (i.e. grammage) and open area of the construct described below. Without being bound by theory, it is believed that these parameters are influenced by the imposed electric field. A driver of fiber deposition is the magnitude and direction of the electrical field generated between the ejector and the collector (Angammana and Jayaram, IEEE Transactions on Industry Applications 2012, 48, 808-815; Kong, et al., Journal of Materials Science 2007, 42, 8106-8112). For example, in a lines pattern, such as depicted in FIG. 6A, it is believed that the electric field forms distinct organized field lines and vectors between the ejector and grounded collector, with small variations near the collector. For example, the more dense and geometrically complex zigzag pattern increases distortion of the electric field, resulting in distorted field lines with fringe effects near the edges of the zigzag pattern of the collector occur. It is believed that this influences polymer jet trajectory, resulting in less overall deposition over the copper trace patterns.

b. Non-Conductive Regions

The collection surface of the collector contains a non-conductive region or a plurality of non-conductive regions. The non-conductive region(s) on the collection surface is defined by the conductive traces. Any suitable non-conductive materials can be used to form non-conductive regions on the collection surface. For example, the non-conductive regions are formed by clay, nylon, polytetrafluoroethylene (Teflon®), PEBAX®, polyurethanes, polyethylene, polypropylene, ceramics, fabrics, or gauze, or a combination thereof.

When the collection surface contains a single conductive trace such as a circle, the non-conductive region is enclosed within the conductive trace.

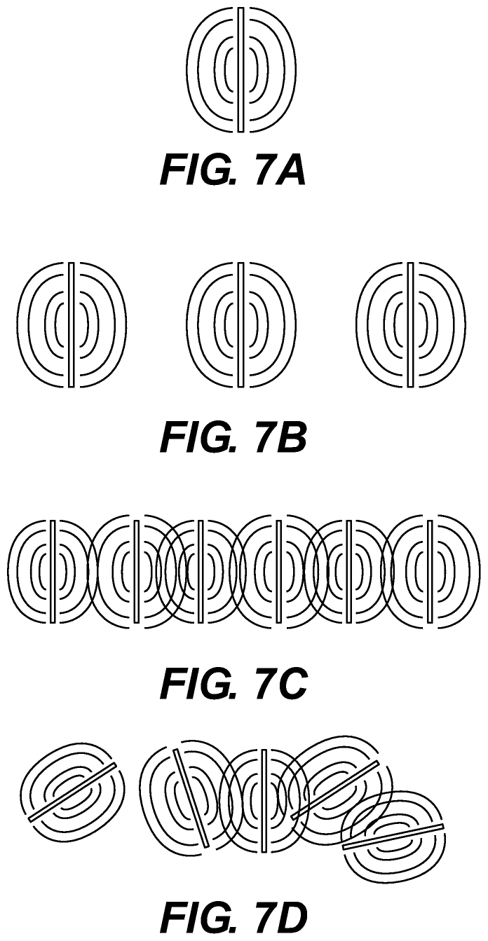

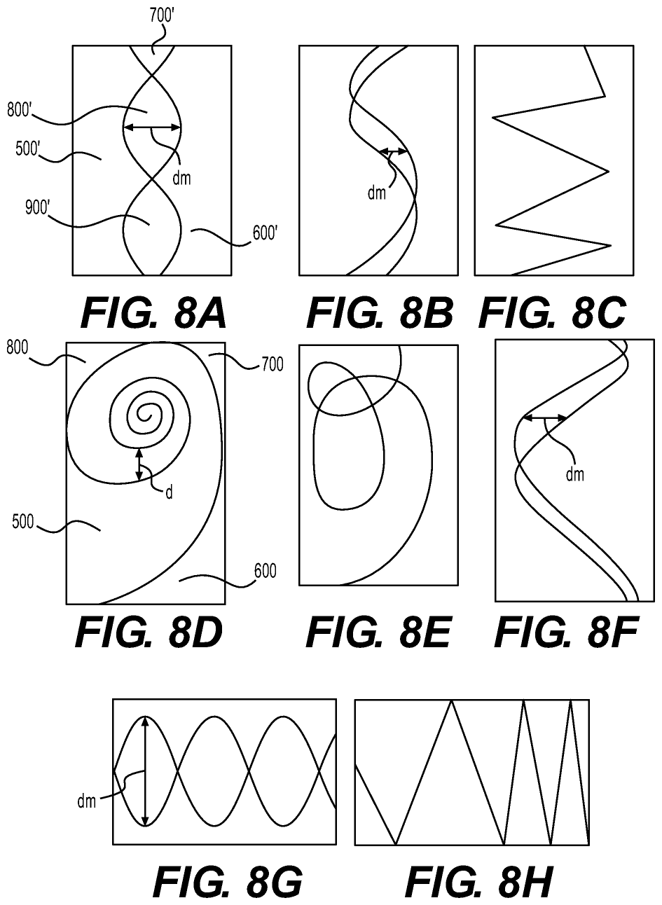

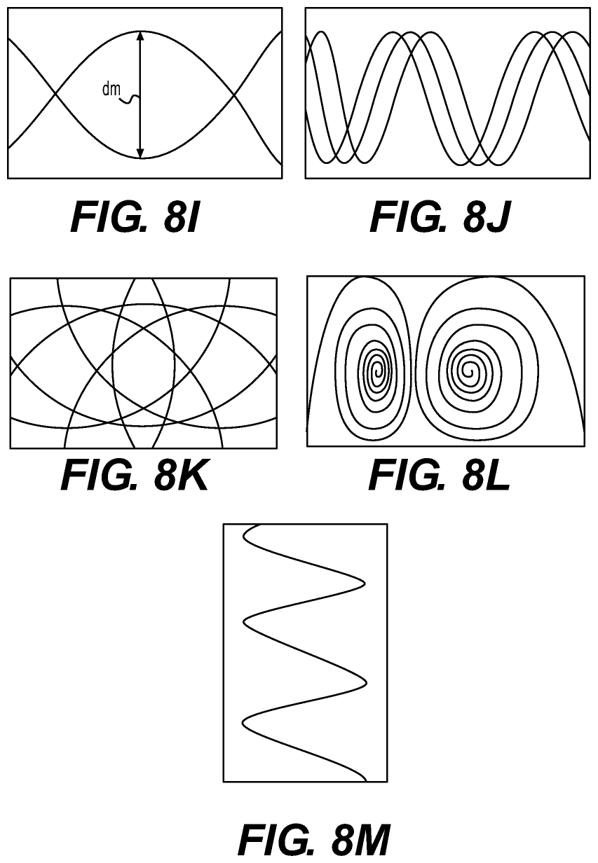

When the collection surface contains a single conductive trace such as a spiral, the collection surface contains non-conductive regions defined by the concentric circles of the spiral (see, e.g., FIG. 8D, 500, 600, 700, and 800). When the collection surface contains a double helix, the collection surface contains non-conductive regions defined by the two helical lines of the double helix (see, e.g., FIG. 8A, 500′, 600′, 700′, 800′, and 900′).

When the collection surface contains a plurality of traces, such as a plurality of straight-lined traces (e.g. FIG. 6A), the collection surface contains a plurality of non-conductive regions with each non-conductive region separated from the adjacent non-conductive region by straight lined trace.

When the collection surface contains a target, the non-conductive region(s) are located inside the boundaries of the target. The boundaries of the target are defined by one or more boundary conductive traces.

c. Exemplary Designs

Exemplary target designs are shown in FIGS. 6A-6D and 8A-8M.

As shown in FIG. 6A, boundary conductive traces 10, 20, 30, and 40 define a target having the shape of a rectangular. A plurality of parallel straight line conductive traces is located within the boundaries. The boundary conductive traces and plurality of parallel conductive traces define a plurality of non-conductive regions. The non-conductive region 51 at the top of the target is defined by the top boundary conductive trace 10, a conductive trace 61, a segment 31a of the left boundary conductive trace 30, and a segment 41a of the right boundary conductive trace 40. The non-conductive region 52 at the bottom of the target is defined by the bottom boundary conductive trace 20, a conductive trace 62, a segment 31b of the left boundary conductive trace 30 and a segment 41b of the right boundary conductive trace 40. Each of the other non-conductive regions in the target is defined by two adjacent conductive traces, a segment of the left boundary conductive trace, and a segment of the right boundary conductive trace. For example, non-conductive region 53 is defined by two adjacent parallel conductive traces 63 and 65, a segment 31c of the left boundary conductive trace 30, and a segment 41c of the right boundary conductive trace 40.

Each of FIGS. 6B-6D shows a portion of the target defined by boundary conductive traces.

FIG. 6B shows a top boundary conductive trace 10′, a portion of a left boundary conductive trace 30′, a portion of a right boundary conductive trace 40′, and three complete conductive traces 81, 82, and 83 in the shape of sinusoidal curves arranged parallel to each other. As shown in FIG. 6B, each of two discrete non-conductive regions 71a and 71b at the top of the target is defined by a segment 11′a and 11′b of the top boundary conductive trace 10′ and the conductive trace 81. The non-conductive region 72 is defined by two adjacent conductive traces 82 and 83, a segment 31′a of the left boundary conductive trace 30′, and a segment 41′a of the right boundary conductive trace 40′.

FIG. 6C shows a top boundary conductive trace 10″, segments of a left boundary conductive trace 30″, segments of a right boundary conductive trace 40″, and a plurality of conductive traces confined therein. The plurality of conductive traces include a first group of parallel straight line conductive trace and a second group of parallel straight line conductive traces. The first group of conductive traces are arranged perpendicular to the second group of conductive traces to form a plurality of conductive traces arranged in a checkerboard pattern. As shown in FIG. 6C, each of two discrete non-conductive regions 271a and 271e at the top of the target is defined by a segment 11″a or 11″e of the top boundary conductive trace 10″, a segment 281a or 281e of a first conductive trace 281, a segment 295a or 295d of the second conductive trace 291 or 294, and a segment 31″a of the left boundary conductive trace 30″ or a segment 41″a of the right boundary conductive trace 40″. Each of the three discrete non-conductive regions 271b-271d at the top of the target is defined by a segment 11″b, 11″c, or 11″d of the top boundary conductive trace 10″, a segment 281b, 281c, or 281d of a first conductive trace 281, and segments of the two adjacent second conductive traces, i.e. segment 295a of trace 291 and segment 295b of trace 292, segment 295b of trace 292 and segment 295c of trace 293, or segment 295c of trace 293 and segment 295d of trace 294. Other non-conductive regions, such as 273b, are defined by segments of two adjacent conductive traces of the first group, i.e. segment 282a of trace 282 and segment 283a of trace 283, and segments of two adjacent conductive traces of the second group, i.e. segment 297a of trace 291 and segment 297b of trace 292.

FIG. 6D shows a top boundary conductive trace 10′″, segments of a left boundary conductive trace 30′″, segments of a right boundary conductive trace 40′″, and four complete zigzag conductive traces 381, 382, 383, and 384 in parallel to each other. As shown in FIG. 6D, non-conductive region 371 at the top of the target is defined by the top boundary conductive trace 10′″, a first conductive trace 381, a segment 31′″a of the left boundary conductive trace 30′″, and a segment 41′″a of the right boundary conductive trace 40″″. Other non-conductive regions, such as non-conductive region 372, are defined by two adjacent conductive traces 381 and 382, a segment 31′″b of the left boundary conductive trace 30′″, and a segment 41′″b of the right boundary conductive trace 40′″.

FIG. 8A shows an exemplary anti-parallel double helix pattern. FIG. 8B shows an exemplary double helix pattern. FIG. 8C shows an exemplary asymmetrical zigzag pattern. FIG. 8D shows an exemplary spiral pattern. FIG. 8E shows an exemplary overlapping circles pattern. FIG. 8F shows an exemplary double helix pattern. FIG. 8G shows an exemplary anti-parallel double helix pattern. FIG. 8H shows an exemplary asymmetrical zigzag pattern. FIG. 8I shows an exemplary anti-parallel double helix pattern. FIG. 8J shows an exemplary overlapped sinusoidal pattern. FIG. 8K shows an exemplary intersecting wavy lines pattern. FIG. 8L shows an exemplary double spiral pattern. FIG. 8M shows an exemplary sinusoidal pattern.

d. Conductive Trace Arrangement

Each parallel conductive trace is at a distance (d) from an adjacent parallel conductive trace (see, e.g. FIGS. 6A-6D, d). Each conductive trace is separated from its adjacent conductive trace(s) via a non-conductive region. The distance between two adjacent parallel conductive traces can be varied based on the size of the collection surface depending on the scale of the collection surface. The distance is selected to produce undisturbed electrostatic field lines or blurred electrostatic field lines aligned around each conductive trace. In some embodiments, each parallel conductive trace is at least a sufficient distance from any adjacent parallel conductive trace to produce undisturbed electrostatic field lines around each of the parallel conductive traces.

Optionally, the distance between two adjacent parallel conductive traces is sufficient to produce undisturbed parallel electrostatic field lines between the ejector and the collector. For example, the distance between two adjacent parallel conductive traces is sufficient to produce electrostatic field lines between the ejector and the collector that do not overlap with one another.

i. Distance

Optionally, the distance between two adjacent parallel conductive traces is at least about 0.3 mm, at least about 0.35 mm, at least about 0.4 mm, at least about 0.45 mm, at least about 0.5 mm, at least about 0.6 mm, at least about 0.65 mm, at least about 0.7 mm, at least about 0.75 mm, at least about 0.8 mm, at least about 0.85 mm, at least about 0.9 mm, at least about 1.0 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, at least about 2.0 mm, at least about 2.1 mm, at least about 2.2 mm, at least about 2.3 mm, at least about 2.4 mm, at least about 2.5 mm, at least about 5 mm, at least about 10 mm, at least about 20 mm, at least about 30 mm, at least about 40 mm, at least about 50 mm, at least about 60 mm, at least about 70 mm, at least about 80 mm, at least about 90 mm, at least about 100 mm, in a range from about 0.3 mm to about 10 cm, from about 0.5 mm to about 10 cm, from about 1 mm to about 10 cm, from about 0.3 mm to about 1 cm, from about 0.5 mm to about 1 cm, or from about 1 mm to about 1 cm, such as about 1.3 mm.

Optionally, the two adjacent parallel conductive traces, such as two adjacent parallel zigzags, saw tooth waves, or sinusoidal curves, can be measured by a shortest distance and a farthest distance (see, e.g. FIGS. 6B and 6D, d₁ and d₂). Exemplary dimensions are noted in FIGS. 6A, 6B, 6C, and 6D. For example, as shown in FIG. 6A, the distance (d) between two adjacent parallel straight lines is 1.30 mm. As shown in FIG. 6B, the two adjacent parallel sinusoidal curves are measured by a shortest distance (d₁) of 0.75 mm and a farthest distance (d₂) of 2.00 mm. As shown in FIG. 6C, the distance (d) between two adjacent parallel straight lines is 1.30 mm. As shown in FIG. 6D, the two adjacent parallel zigzags are measured by a shortest distance (d₁) of 0.35 mm and a farthest distance (d₂) of 0.60 mm. However, these dimensions can be the same or different, as needed, to form a desired construct on a particular scale. In these embodiments, the shortest distance between the two adjacent parallel conductive traces is at least about at least about 0.3 mm, at least about 0.35 mm, at least about 0.4 mm, at least about 0.45 mm, at least about 0.5 mm, at least about 0.6 mm, at least about 0.65 mm, at least about 0.7 mm, at least about 0.75 mm, at least about 0.8 mm, at least about 0.85 mm, at least about 0.9 mm, at least about 1.0 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, at least about 2.0 mm, at least about 2.1 mm, at least about 2.2 mm, at least about 2.3 mm, at least about 2.4 mm, at least about 2.5 mm, at least about 5 mm, at least about 10 mm, at least about 20 mm, at least about 30 mm, at least about 40 mm, at least about 50 mm, at least about 60 mm, at least about 70 mm, at least about 80 mm, at least about 90 mm, at least about 100 mm, in a range from about 0.3 mm to about 10 cm, from about 0.5 mm to about 10 cm, from about 1 mm to about 10 cm, from about 0.3 mm to about 1 cm, from about 0.5 mm to about 1 cm, or from about 1 mm to about 1 cm, such as about 1.3 mm.

When the collection surface contains a single conductive trace, such as a spiral, the concentric circles of the spiral are separated by non-conductive regions such that each concentric circle is at a distance d from its adjacent concentric circle (see, e.g. FIG. 8D, d). When the collection surface contains a double helix, the two helical lines have intersection points and are also separated by non-conductive regions such that each helical line has a farthest distance dm from the other helical line of the double helix (see, e.g. FIGS. 8A, 8B, 8F, 8G, and 8I, dm).

e. Percentage of Conductive Trace Area

Typically, in the pattern on the collection surface the conductive trace area is from about 5% to about 99% of the total area of the target. The term “total area of the target” refers to the total exposed areas of a target defined by two or more boundary conductive traces on the collection surface. The total area of the target includes both conductive and non-conductive areas enclosed within the boundaries defining the target. For example, as shown in FIG. 6A, boundary conductive traces 10, 20, 30, and 40 define a target having a shape of rectangular. The total area of such a target is calculated by W₁×L₁. The values shown for W₁ (10.0 mm) and L₁ (30.0 mm) in FIG. 6A are exemplary. These dimensions can be the same or different, as needed, to form a desired construct on a particular scale.

Optionally, the conductive trace area is from about 5% to about 95% of the total area of the target, from about 5% to about 90%, from about 5% to about 85%, from about 5% to about 80%, from about 5% to about 75%, from about 5% to about 70%, from about 10% to about 95%, from about 10% to about 90%, from about 10% to about 85%, from about 10% to about 80%, from about 10% to about 75%, from about 10% to about 70%, from about 15% to about 95%, from about 15% to about 90%, from about 15% to about 85%, from about 15% to about 80%, from about 15% to about 75%, from about 15% to about 70%, from about 15% to about 65%, from about 15% to about 60%, from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 25% to about 95%, from about 25% to about 90%, from about 25% to about 85%, from about 25% to about 80%, from about 25% to about 75%, from about 25% to about 70%, from about 25% to about 65%, from about 25% to about 60%, from about 30% to about 95%, from about 30% to about 90%, from about 30% to about 85%, from about 30% to about 80%, from about 30% to about 75%, from about 30% to about 70%, from about 30% to about 65%, from about 30% to about 60%, from about 35% to about 95%, from about 35% to about 90%, from about 35% to about 85%, from about 35% to about 80%, from about 35% to about 75%, from about 35% to about 70%, from about 35% to about 65%, from about 35% to about 60%, from about 40% to about 95%, from about 40% to about 90%, from about 40% to about 85%, from about 40% to about 80%, from about 40% to about 75%, from about 40% to about 70%, from about 40% to about 65%, or from about 40% to about 60%, such as from about 40% to about 75%, from 40% to about 65%, from about 44% to about 74% or from about 44% to about 64% of the total area of the target.

### 3. Optional Components

Optionally, the electrospinning system also includes a power supply, a pressurization component, a temperature control component, a position control component, one or more sensors, a controller, an output device, or a combination thereof.

a. Power Supply

Optionally, the electrospinning system includes a power supply, such as a direct current power supply. The power supply can generate a high voltage, such as up to 30 kV, up to 25 kV, up to 20 kV, up to 15 kV, or up to 12 kV.

The power supply is connected to the ejection device or one or more components thereof. For example, the power supply is connected to the reservoir, the ejector, or both the reservoir and the ejector.

When in use, the power supply applies an electrostatic charge to the ejection device or one or more component thereof to generate an electrostatic field. When the electrostatic field intensity is sufficiently strong, forces on the surface of the spinning dope (e.g. a polymer solution) at the tip of the ejector (e.g. a needle or nozzle) overcome the surface tension; the surface of the spinning dope elongates and makes a jet in the direction of the applied field.

b. Pressurization Component

Optionally, the electrospinning system includes a pressurization component, such as a pressurized air tank or a vacuum pump. The pressurization component can be connected to the reservoir such that when in use, it applies pressure and/or vacuum to the spinning dope contained within the reservoir to push the spinning dope to the tip portion of the ejector.

For example, a pressurized air tank and/or vacuum pump is connected to the reservoir via a connecting tube(s) such that when in use, pressurized air and/or vacuum is supplied to the reservoir though the connecting tube. The supplied pressurized air and/or vacuum pushes the polymer solution within the reservoir to the tip portion of the ejector attached thereto.

c. Temperature Control Component

Optionally, the electrospinning system includes a temperature control component. The temperature control component can maintain the reservoir and the spinning dope at a predetermined temperature or vary the temperature of the reservoir and the spinning dope, and accordingly the viscosity of the spinning dope can be maintained or varied.

Typically, the temperature control component is arranged such that when in use, heating or cooling is provided to the exterior of the reservoir. For example, heating coils or electrical heat tracing cables are wrapped around the exterior of the reservoir to maintain or vary the temperature of the reservoir and spinning dope. For example, a tubing is wrapped around the exterior of the reservoir, where a heating or cooling medium is running through the tubing to maintain or vary the temperature of the reservoir and spinning dope.

d. Position Control Component

Optionally, the electrospinning system includes a position control component. The position control component can adjust the distance and/or alignment between the ejection device and the collection surface.

For example, the ejection device and/or collector is placed on top of a movable stage. In use, the stage can move vertically or in any position parallel to a horizontal plane to increase or decrease the distance and/or change the alignment between the ejection device and the collection surface.

For example, the ejection device and/or collector is connected to a movable arm. When in use, the stage can move vertically or in any position parallel to a horizontal plane to increase or decrease the distance and/or change the alignment (e.g. distance and/or angle) between the ejection device and the collection surface.

e. Sensors

Optionally, the electrospinning system includes one or more sensors to measure one or more parameters of the system when in use, such as pressure and/or temperature inside the reservoir, position of the ejection device and/or collector, and the voltage provided to the ejection device. For example, the electrospinning system includes a pressure sensor, a temperature sensor, a position sensor, or a voltage sensor, or a combination thereof. The sensor can be connected to the ejection device or a component thereof, or to the collector, or a combination thereof.

For example, a pressure and/or temperature sensor is connected to the reservoir to measure the pressure and/or temperature inside the reservoir such that these parameters can be controlled to maintain a set value or set range to provide a steady supply of the spinning dope to the tip portion of the ejector.

For example, a sensor is connected to the ejection device or a component thereof to measure the position of the ejection device. For example, a sensor is connected to the collector to measure the position of the collector. For example, a first sensor is connected to the ejection device or a component thereof and a second sensor is connected to the collector to measure the position of the ejection device and the collector.

For example, a sensor is connected to the ejection device or a component thereof to measure the applied voltage.

Optionally, the sensor includes a processor. Optionally, the processor can transmit one or more signals or data to a controller by a wireless transmitter. Optionally, the processor can receive one or more signals or data from the controller and accordingly vary one or more parameters of the system to a predetermined set point by a user. For example, the processor can perform one or more algorithms to maintain or vary the desired parameter at a predetermined set value or range.

f. Controller and Output Device

Optionally, the electrospinning system includes a controller. The controller contains a transmitter, such as a wireless transmitter, that can receive output(s) from the processor of the sensor.

Optionally, the controller is connected to a display to allow a user to make adjustments to any one of the set points to move the desired measured or controlled parameter to the set point.

### B. Configuration of Electrospinning System

Generally, when in use, the collector is located a distance away from the ejection device and aligned with the ejection device. The collection surface of the collector is faces the ejection device and configured to receive the ejecting polymer solution. The collector is located at an angle and a distance relative to the ejection device such that the ejecting polymer solution can contact the collection surface of the collector. When two or more collectors are included in the system, each collector can be located at an angle and a distance relative to the ejection device and to each other such that the ejecting polymer solution can reach the collection surface of each collector.

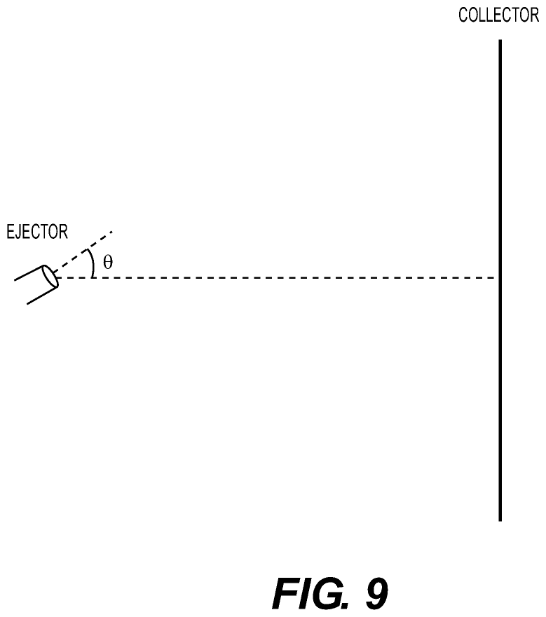

The collector can be located at an angle (θ) between a horizontal axis of the collector that is perpendicular to the collection surface and an imaginary longitudinal line running through the center of the tip of the ejector (see, e.g. FIG. 9, θ). The angle (θ) can be in a range from 0° to 90°, from 0° to 85°, from 0° to 80°, from 0° to 75°, from 0° to 70°, from 0° to 65°, from 0° to 60°, from 0° to 55°, from 0° to 50°, from 0° to 45°, from 0° to 40°, from 0° to 35°, from 0° to 30°, from 0° to 25°, from 0° to 20°, from 0° to 15°, from 0° to 10°, or from 0° to 5°, such as about 0°.

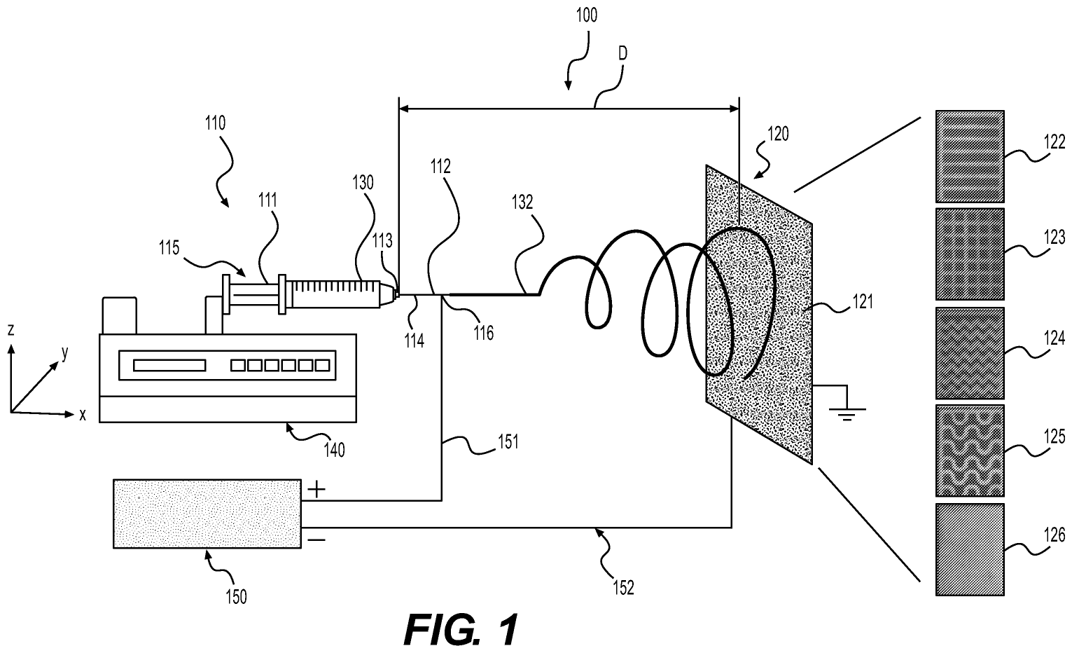

The distance between the collector and the ejection device is measured by an imaginary horizontal line running from the tip of the ejector and the collection surface of the collector (see, e.g. FIG. 1, D). The distance can be a suitable distance. For example, the distance can be in a range from about 5 cm and about 100 cm, from about 5 cm and about 90 cm, from about 5 cm and about 80 cm, from about 5 cm and about 70 cm, from about 5 cm and about 60 cm, from about 5 cm and about 50 cm, from about 5 cm and about 40 cm, from about 5 cm and about 30 cm, from about 5 cm and about 20 cm, such as about 15 cm.

For example, the collector is located at an angle (θ) in a range from 0° to 40°, from 0° to 20°, or from 0° to 10° between a horizontal axis of the collector and an imaginary longitudinal line running through the center of the tip of the ejector and at a distance from the ejection device in a range from about 5 cm to about 50 cm, from about 5 cm to about 25 cm, or from about 10 cm to about 20 cm, such as about 15 cm.

The height of the ejector where the polymer jet starts can be the same as or different from the height(s) of the collector(s). The tip of the ejector can align with any point on the collection surface. For example, the tip of the ejector aligns with the center of the collection surface, a point towards the top of the collection surface, or a point towards the bottom of the collection surface.

The tip of the ejector can be pointed in a horizontal orientation or at an angle (θ′) less than about 90° relative to the horizontal level, such as an angle that is about 10°, 20°, 30°, 40°, 50°, 60°, 70°, or 80° relative to the horizontal level.

### 1. Exemplary Configuration

An exemplary electrospinning system is illustrated in FIG. 1. As shown in FIG. 1, an electrospinning system 100 includes an ejection device 110 and a collector 120. The collector 120, such as an etched PCB collector, contains a collection surface 121 that is exposed to the ejecting polymer. The collection surface 121 can contain different patterns, such as lines 122, squares 123, zigzags 124, or sinusoids 125, depending on the desired electrostatic field distribution. A solid plate 126 can be used as a control. The ejection device 110 includes a reservoir 111 and a needle 112 attached thereto. When in use, the reservoir 111 holds a polymer solution 130. The needle 112 is attached to a longitudinal end 113 of the reservoir via a connecting portion 114 of the ejector. When in use, the polymer solution 130 is extruded and/or drawn by an electrostatic force through the needle 112 and the polymer jet 132 towards the collection surface 121.

The reservoir 111 is a syringe that can be sealed in a pressure-tight manner thorough a plunger flange 115. The reservoir 111 is mounted on a syringe pump 140 that can apply pressure to the polymer solution 130 by pushing the plunger flange 115 of the syringe towards the needle 112. The applied pressure delivers the polymer solution 130 from the reservoir to the tip 116 of the needle. The syringe pump 140 can also move the ejector in the x, y, or z direction. Moving along an x-direction can position the ejector closer or farther away from the collector. Moving along a y-direction can change the alignment of the ejector with the collector in the horizontal axis. Moving along a z-direction can change the alignment of the ejector with the collector in the vertical axis.

A power supply 150 is connected to the tip portion of the needle via a wire 151 and to the collector 152 such that an electrostatic charge to the ejection device 110 while the collector is grounded.

### II. Methods of Making the Electrospun Fiber Constructs

Methods of generating electrospun fiber constructs using the electrospinning system are described herein. The terms “electrospun fiber construct” and “fiber construct” are used interchangeably herein.

Generally, the method includes (i) applying an electrostatic charge to the ejection device or a component thereof. The ejection device includes a reservoir and an ejector, where a polymer solution is contained within the reservoir. Following step (i), the polymer solution is extruded out of the ejection device via the ejector. The ejector is configured to direct the polymer solution towards the collection surface of the collector. During ejection, the polymer solution is further directed to form patterned fiber constructs via the electrostatic field lines between the ejector and the collection surface. Typically, the formed fiber construct has a grammage that is at least 5% higher than a grammage of a fiber construct formed on an un-patterned collector under the same conditions. The term “un-patterned” refers to a collector that has a collection surface containing a uniform surface, i.e. a solid surface that has no pattern on it.

Optionally, the ejection device includes at least two reservoirs, where a polymer solution is contained within at least a first reservoir and an active agent solution or suspension is contained within at least a second reservoir. The first and second reservoirs can attach to the same ejector or to two different ejectors. In these embodiments, the active agent solution or suspension is extruded simultaneously with or intermittently with the polymer solution to form an electrospun fiber construct on the collection surface. The active agent can be encapsulated within and/or coated on the surface of the formed electrospun fiber construct.

The term “grammage” refers to the density of a porous fiber construct. Grammage is area density (μg/mm²), which is calculated as follows:

Fiber Grammage=fiber scaffold mass÷fiber(scaffold) area

The same conditions means that the fiber construct is generated using the same operation parameters, such as the same ejection device, same polymer concentration, same solvent, same polymer molecular weight, same applied voltage, same distance to target, same alignment between the ejection device and the collector, and same environmental conditions (e.g. temperature, humidity, pressure).

Optionally, the method of generating fiber constructs using the electrospinning system includes the step of selecting a collector, wherein the collector has a pattern sufficient to generate the fiber construct having a grammage that is at least 5% higher than a grammage of a fiber construct formed on an un-patterned collector under the same conditions, and (i) applying an electrostatic charge to the ejection device or a component thereof. The ejection device includes a reservoir and an ejector, where a polymer solution is contained within the reservoir. Following step (i), the polymer solution is extruded out of the ejection device via the ejector. The ejector directs the polymer solution towards the collection surface of the collector. During ejection, the electrostatic field lines between the ejector and the collection surface guide the polymer solution to form patterned fiber constructs.

Optionally, the method is performed inside a sterile environment, i.e. the electrospinning system is placed inside a sterile environment such as a traditional cell culture hood, allowing for sterile generation of electrospun fiber constructs.

### A. Applying Electrical Voltage to Injection Device

Typically, a user applies an electrostatic charge to the ejection device or a component thereof using a power supply. The power supply is connected to both the ejection device or a component of the ejection device and the collector via conductive wires. Typically, the collector is grounded. For example, the electrostatic charge is applied to the ejector or a portion of the ejector (e.g. the tip portion of the ejector) of the ejection device via a conductive wire.

### 1. Electrostatic Charge

The electrostatic charge applied to the ejection device or a component thereof is sufficient to generate an electrostatic field. When the electrostatic field intensity is sufficiently strong (i.e. reaches a threshold electrostatic field intensity), forces on the surface of the polymer solution at the tip of the ejector (e.g. a needle or nozzle) overcome the surface tension; the surface of the polymer solution elongates and forms a jet of the polymeric solution in the direction of the applied field.

The threshold electrostatic field intensity depends on many parameters, such as the type of polymer and solvent, the solution concentration and viscosity, and the temperature of the system. These parameters can be varied to form polymeric fiber constructs having the desired properties, e.g. strength, flexibility, thickness, hydrophobicity/contact angle, etc.

Optionally, the electrostatic charge applied to the ejection device or a component thereof to generate an electrostatic field intensity is sufficient to overcome the surface tension of polymer solutions is in a range from about −15 kV to about 30 kV, from about −15 kV to about 20 kV, from about −10 kV to about 30 kV, from about −10 kV to about 20 kV, from about −5 kV to about 30 kV, from about −5 kV to about 20 kV, from about 0 to about 30 kV, from about 0 to about 20 kV, from about 0 to about 15 kV, such as about 12 kV.

### 2. Polymer Solutions

When a polymer solution is in the reservoir of the ejection device. The polymer solution contains one or more polymers for forming the fiber construct and a solvent. The polymer solution can further contain an additive, optionally more than one additive or an active agent, optionally more than one active agent or a combination thereof.

Optionally, the ejection device includes more than one reservoir. In such embodiments, each reservoir can contain a polymer solution that is the same as or different from the other reservoirs. For example, the ejection device includes two or more reservoirs, where at least two reservoirs contain the same polymer solution and at least one reservoir contains a polymer solution that is different from the other reservoirs.

Following applying an electrostatic charge to the ejection device or a component thereof using a power supply, the polymer solution is extruded out of the ejection device via the ejector at an extrusion rate. The extruded polymer solution forms a jet that is directed to form patterned fiber constructs via the electrostatic field lines between the ejector and the collection surface.

a. Polymers

Generally, the polymer used for generating the fiber constructs is a biocompatible polymer. The term “biocompatible” refers to a material that is, with any metabolites or degradation products thereof, generally non-toxic to the recipient, and cause no significant adverse effects to the recipient. Generally, biocompatible polymers do not elicit a significant inflammatory or immune response when administered to a patient. Optionally, a biocompatible polymer elicits no detectable change in one or more biomarkers indicative of an immune response. For example, a biocompatible polymer elicits no greater than a 10% change, no greater than a 20% change, or no greater than a 40% change in one or more biomarkers indicative of an immune response.

The biocompatible polymer used for generating the fiber construct can be biodegradable or non-biodegradable. The term “biodegradable” refers to material, structure, device, or device component that degrades or breaks down into its component subunits, or digestion products, e.g., by a biochemical process, of the material into smaller (e.g., non-polymeric) subunits. Optionally, a biodegradable polymer, structure, device, or device component degrades into CO₂, H₂O, and other biomass materials. Optionally, the degradation of a biodegradable polymer forming the fiber construct occurs over a period less than 30 days, less than 60 days, less than 90 days, less than 120 days, less than 180 days, less than 1 year. Optionally, the degradation of a biodegradable polymer forming the fiber construct occurs over a period greater than 30 days, greater than 60 days, greater than 90 days, greater than 120 days, greater than 180 days, or greater than 1 year. Optionally, degradation of a polymer, structure, device, or device component is said to be complete when at least 80% by mass has degraded, when at least 85% by mass has degraded, when at least 90% by mass has degraded, when at least 95% by mass has degraded, or when at least 99% by mass has degraded. The biodegradation rate of the polymer, structure, device or device component depends upon several factors, both environmental and material. Non-limiting examples of environmental factors influencing biodegradation rates include temperature, pH, oxygen concentrations, and microbial and enzymatic activities. Non-limiting examples of material properties influencing biodegradation rates of the polymer, structure, device or device component include degree of branching of the polymer chains, the presence and amount of hydrophilic groups, stereochemistry, molecular weight, the degree of crystallinity, the crosslinking, surface roughness, and the surface to volume ratio.

For application to tissues to prevent inflammation, enlargement and/or over-proliferation, polymers degrading substantially within two months, six months, or twelve months after implantation can be used. For prevention of adhesions or controlled release, the time over which degradation occurs should be correlated with the time required for healing, e.g., in excess of two weeks but less than six months.

Suitable biocompatible polymers for forming the electrospun fiber construct are commercially available or readily synthesizable using methods known to those skilled in the art. These polymers include: soluble and insoluble, biodegradable and non-biodegradable natural or synthetic polymers. These can be hydrogels or thermoplastics, homopolymers, copolymers or blends, natural or synthetic. The term “hydrogel” refers to an aqueous phase with an interlaced polymeric component, preferably with 90% of its weight as water.

i. Exemplary Polymers

Exemplary biocompatible polymers for preparing the polymer solution to form the electrospun fiber construct include natural polymers, synthetic polymers, and a combination thereof. The polymers may be selected, modified, or combined to achieve a desired property of the formed electrospun fiber construct, such as a desired mechanical property, bioactivity and/or biocompatibility, degradation rate, and hemocompatibility.

### (1) Natural Polymers

Representative natural polymers suitable for use in the polymer solution include proteins, such as zein, modified zein, casein, gelatin, gluten, serum albumin, elastin, fibronectin, fibrin, or collagen, and polysaccharides, such as cellulose, alginate, and other polysaccharides including dextran and cellulose, collagen, dextrans, hyaluronic acid, polymers of acrylic and methacrylic esters and alginic acid, and cellulosics. Natural gums may be utilized to form the fiber construct, such as Guar gum, carageenen, Okra gum, locust bean gum, honey locust gum, tara gum, sterculia foetida, khaya gum, and other natural gums, such as described in Avichat el al., “Recent Investigations of Plant Based Natural Gums, Mucilages and Resins in Novel Drug Delivery Systems, Ind. J. Pharm. Edu. Res., 24(a):86-99 (2010). These may be utilized, although they are somewhat less desirable due to higher levels of variability in the characteristics of the final products, and in degradation following administration. Synthetically modified natural polymers include alkyl celluloses, hydroxyalkyl celluloses, cellulose ethers, cellulose esters, and nitrocelluloses, acrylic or methacrylic esters of above natural polymers to introduce unsaturation into the biocompatible polymers.

### (2) Synthetic Polymers

Representative synthetic polymers suitable for forming the fiber construct are: poly(hydroxy acids) such as poly(lactic acid), poly(glycolic acid), and poly(lactic acid-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-coaprolactone), derivatives, copolymers and blends thereof. Synthetic polymers can include polyesters, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polysiloxanes, polyurethanes and copolymers thereof. Other polymers include celluloses such as methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, acrylates such as poly(methyl methacrylate), poly(ethyl methacrylate), poly(butyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(vinyl acetate), polyvinyl chloride, polystyrene, polyvinyl pyrrolidone, and polyvinylphenol. Representative bioerodible polymers include polylactides, polyglycolides and copolymers thereof, poly(hydroxy butyric acid), poly(hydroxyvaleric acid), poly(lactide-co-caprolactone), poly[lactide-co-glycolide], polyanhydrides, polyorthoesters, derivatives, blends and copolymers thereof. As used herein, “derivatives” include polymers having substitutions, additions of chemical groups, for example, alkyl, alkylene, hydroxylations, oxidations, and other modifications routinely made by those skilled in the art.

Optionally, the synthetic polymer suitable for forming the fiber construct is polyhydroxyalkanoate, polyglycerol sebacate, polyvinyledene fluoride (“PVDF”), PVDF-co-HFP, terpolymer of PTFE-co-PVDF-co-HFP, polyethyleneterephthalate (PET), polyvinylchloride (PVC), polyesters such as poly(ethylene terephthalate), polyamides such as nylon, polyacrylonitriles, polyphosphazines, polylactones such as polycaprolactone, polyanhydrides such as polybis(p-carboxphenoxy)propane anhydride, polyalkylsulfones, polycarbonate polymers and copolymers, polyhydroxybutyrates, polyhydroxyvalerates and their copolymers, polyurethanes, hydrocarbon copolymers, polyethylene, polypropylene, polyvinyl chloride and ethylene vinyl acetate, polyorthoesters, polyanhydrides, polytyrosine, PHBV, polylactide, polylactic acid, polyglycolide, copolymers of lactide and glycolide (“PLGA”), polyhydroxybutyrate, copolymers of lactic acid and lactone, copolymers of lactic acid and PEG (“PLLA-co-PEG”), copolymers of a-hydroxy acids and C-amino acids (polydepsipeptides), polyanhydrides, polyorthoesters, polyphosphazenes, copolymers of hydroxybutyrate and hydroxyvalerate, homopolymers and copolymers of delta-valerolactone, p-dioxanone, and their copolymers with caprolactone, poly (ethylene carbonate), copoly(ethylene carbonate), polyethylene terephthalate, polycaprolactone (“PCL”), poly(acrylic acids) (i.e., Carbopols™), poly(acrylates), polyacrylamides, polyvinyl alcohols, polyethylene glycols, polyethylene vinyl acetates, poly(oxyalkene) polymers and copolymers such as poly(ethylene oxide)-poly(propylene oxide) (PEO-PPO) copolymers, and copolymers and blends of these polymers with polymers such as poly(alpha-hydroxy acids), including but not limited to lactic, glycolic and hydroxybutyric acids, polycaprolactones, or polyvalerolactones, or a copolymer thereof.

For example, the synthetic polymer for forming the fiber construct is lactide homopolymers, such as poly(L-lactide), poly(D,L-lactide), copolymers of lactide and glycolide (“PLGA”), poly(oxyethylene oxide)(“PEO”), poly(oxyethylene)-poly(oxypropylene), PEO-PPO block copolymers such as triblock PEO-PPO-PEO copolymers (POLOXAMERS™ PLURONICS™), poly (ethylene oxide)-poly(butylene oxide) (PEO-PBO), tetra-functional block copolymers derived from the sequential addition of propylene oxide and ethylene oxide to ethylene diamine (POLOXAMINES™, TETRONICS™), copolymers of PEG with poly(lactic acid), oligomers of poly(lactic acid), lactides, copolymers of PEG and amino acids, or conjugates of PEG with polysaccharides, or a copolymer thereof.

Examples of biodegradable synthetic polymers suitable for use in the polymer solution polymers include polymers of hydroxyacids such as lactic acid and glycolic acid (e.g. poly(lactic-co-glycolic acid)), and copolymers with PEG, polyanhydrides, poly(ortho)esters, polyurethanes, poly(butyric acid), poly(valeric acid), poly(lactide-coaprolactone), blends and copolymers thereof. Examples of preferred non-biodegradable polymers include ethylene vinyl acetate, poly(meth)acrylic acid, polyamides, copolymers and mixtures thereof. The biodegradable polymers degrade either by enzymatic hydrolysis or exposure to water in vivo, or by surface or bulk erosion.

These polymers can be obtained from sources such as Sigma Chemical Co., St. Louis, MO., Polysciences, Warrenton, PA, Aldrich, Milwaukee, WI, Fluka, Ronkonkoma, NY, and BioRad, Richmond, CA. or else synthesized from monomers obtained from these suppliers using standard techniques.

ii. Molecular Weight

Generally, the polymer used for forming the fiber construct has a molecular weight suitable to form a polymer solution that can be ejected from the ejection device. The molecular weight of the polymer for forming the fiber construct can be selected based on the specific polymer and the solvent.

Optionally, the polymer used for forming the fiber construct has a molecular weight in a range from about 1 kDa (i.e. 1×10³ g/mol) to about 500 kDa (i.e. 500×10³ g/mol), from about 1 kDa to about 400 kDa, from about 1 kDa to about 300 kDa, from about 1 kDa to about 200 kDa, from about 1 kDa to about 100 kDa, from about 1 kDa to about 50 kDa, from about 1 kDa to about 10 kDa, from about 5 kDa to about 500 kDa, from about 5 kDa to about 400 kDa, from about 5 kDa to about 300 kDa, from about 5 kDa to about 200 kDa, from about 5 kDa to about 100 kDa, from about 5 kDa to about 50 kDa, from about 10 kDa to about 500 kDa, from about 10 kDa to about 400 kDa, from about 10 kDa to about 300 kDa, from about 10 kDa to about 200 kDa, or from about 10 kDa to about 100 kDa.

b. Solvents

When a polymer solution is used to generate a jet towards the collector and form the fiber construct on the collection surface, a solvent is used to dissolve the polymer. The solvent suitable for dissolving the polymer forming the fiber construct can be an organic solvent, an aqueous solvent, or a combination thereof.

Exemplary solvents that can dissolve a biocompatible polymer described above to form a polymer solution include, but are not limited to, acetone, dimethylformamide, dimethylsulfoxide, N-methylpyrrolidone, N, N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, and alcohol ionic compounds.

Optionally, the solvent is a mixture of two or more of these exemplary organic solvents. Optionally, the solvent is a mixture of one or more of these exemplary organic solvents and water.

When an active agent and/or an additive is added in the polymer solution, the solvent can dissolve the active agent and/or additive or form a suspension of the active agent and/or additive. For example, the active agent has a solubility that is similar to the polymer and dissolves in the solvent. Alternatively, the active agent is insoluble in the solvent dissolving the polymer and thus suspended in the solvent. For example, the additive has a solubility that is similar to the polymer and dissolves in the solvent. Alternatively, the additive is insoluble in the solvent dissolving the polymer and thus suspended in the solvent. When the polymer solution contains both an active agent and an additive, the active agent can have the same or similar solubility as the additive, or a solubility that is different from the additive.

c. Active Agents and/or Additives

The polymer solution can include an active agent, optionally more than one active agent, and/or an additive, optionally more than one additive. Optionally, the active agent, optionally more than one active agent are mixed with a solvent described above to form an active agent solution or suspension. The active agent solution or suspension can be loaded in a second reservoir that is different from the reservoir holding the polymer solution and the two reservoirs can attach to the same ejector or to two different ejectors. The active agent is extruded simultaneously with or intermittently with the polymer solution to form an electrospun fiber construct on the collection surface. The active agent can be encapsulated within or coated on the surface of the formed electrospun fiber construct. Optionally, one or more active agent(s) is added to the formed electrospun fiber construct in a post-processing step as described below.

i. Active Agents

One or more active agents can be added in the polymer solution to form the fiber construct. Examples of suitable active agents include therapeutic, prophylactic, nutraceutical, and diagnostic agents. These can include organic compounds, inorganic compounds, cells, antibodies, proteins, carbohydrates, polysaccharides, lipids, nucleic acids, steroids, hormones, or any other suitable materials.

Optionally, active agents suitable for use in the polymer solution include, but not limited to, anti-fibrotic/anti-scarring agents, anti-inflammatory agents, anti-infectious agents, growth factors, anti-cancer agents (e.g. antimitotic agents, anti-proliferative agents, etc.), anesthetic agents, prothrombotic/antithrombotic agents, and anti-allergenic agents, and a combination thereof.

### (1) Anti-Fibrotic/Anti-Scarring Agents

Exemplary anti-fibrotic/anti-scarring agents suitable for use in the polymer solution include, but are not limited to, Bleomycin (e.g. Blenoxane®), interferon, steroids, and botulinum toxins.

### (2) Anti-Inflammatory Agents

Exemplary anti-inflammatory agents suitable for use in the polymer solution include, but are not limited to, triamcinolone acetonide, fluocinolone acetonide, prednisolone, dexamethasone, loteprendol, fluorometholone, ketorolac, nepafenac, and diclofenac.

### (3) Anti-Infectious Agents

Exemplary anti-infectious agents suitable for use in the polymer solution include, but are not limited to, antiviral agents, antibacterial agents, antiparasitic agents, and anti-fungal agents. Exemplary antibacterial agents such as antibiotics include moxifloxacin, ciprofloxacin, erythromycin, levofloxacin, cefazolin, vancomycin, tigecycline, gentamycin, tobramycin, ceftazidime, ofloxacin, gatifloxacin; antifungals: amphotericin, voriconazole, natamycin.

### (4) Growth Factors

Exemplary growth factors, such as epidermal growth factors (EGF), suitable for use in the polymer solution include, but are not limited to, heparin-binding EGF-like growth factor, amphiregulin, epiregulin, epigen, betacellulin, neuregulin-1, neuregulin-2, neuregulin-3, neuregulin-4, transforming growth factor-alpha (TGF-α), transforming growth factor-beta (TGF-β), platelet-derived growth factors (PDGF), fibroblast growth factors (FGF), including FGF acidic isoforms 1 and 2, FGF basic form 2 and FGF 4, 8, 9 and 10, nerve growth factors (NGF) including NGF 2.5 s, NGF 7.0 s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors, and a combination thereof.

### (5) Anti-Cancer Agents

Exemplary anti-cancer agents that are suitable for the polymer solution include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel, epothilones A-F, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-ß inhibitors, and antibodies to the epidermal growth factor receptor such as panitumumab (VECTIBIX®) and cetuximab (ERBITUX®).

Other suitable anti-neoplastic agents include cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, piposulfan, altretamine, asparaginase, bleomycin, busulfan, carboplatin, carmustine, chlorambucil, cisplatin, cladribine, cyclophosphamide, cytarabine, dacarbazine, diethylstilbestrol, ethinyl estradiol, etoposide, mitomycin, mitotane, mitoxantrone, paclitaxel, pentastatin, pipobroman, plicamycin, prednisone, procarbazine, streptozocin, tamoxifen, teniposide, vinblastine, and vincristine.

Optionally, the polymer solution contains one or more anti-cancer agents that are anti-cancer antibodies, such as alemtuzumab, bevacizumab, panitumumab, cetuximab, ofatumumab, trastuzumab, rituximab, arcitumomab, ibritumomab tiuxetan, capromab pendetide, tositumomab, ipilimumab, tremelimumab, nivolumab, pembrolizumab (MK-3475), pidilizumab, MEDI0680, PDR001, atezolizumab (MPDL33280A), durvalumab (MEDI4736), avelumab, BMS986016, LAG525, lirilumab, monalizumab, IPH4102, TSR-022, CM-24 (MK-6018), or MEDI9447, or a combination thereof.

### (6) Anesthetic Agents

Exemplary anesthetic agents suitable for use in the polymer solution include, but are not limited to, analgesics (e.g. acetaminophen, non-steroidal anti-inflammatory, narcotics, etc.), anxiolytics, local anesthetics, general anesthetics (e.g. Propofol (Diprivan®), Ketamine, Etomidate, etc.), and paralytics (e.g. Zemuron® and Nimbex®).

### (7) Prothrombotic/Antithrombotic Agents

Exemplary prothrombotic/antithrombotic agents suitable for use in the polymer solution include, but are not limited to, thrombolytics (e.g. alteplase, streptokinase, urokinase, alteplase, urokinase, reteplase, and tenecteplase), antiplatelet agents (e.g. aspirin, dipyridamole, thienopyridines, pentoxifylline, cilostozol, and cyclo-pentyl-triazolo-pyrimidine (CPTP) inhibitors, such as ticagrelor), anticoagulants, and thrombolytic agents, such as direct Xa inhibitors, heparin, and warfarin.

### (8) Anti-Allergenic Agents

Exemplary anti-allergenic agents suitable for use in the polymer solution include, but are not limited to, antihistamine (e.g. azelastine, carbinoxamine, cyproheptadine, desloratadine, emedastine, hydroxyzine, levocabastine, levocetirizine, brompheniramine, cetirizine, chlorpheniramine, clemastine, diphenhydramine, fexofenadine, and loratadine), decongestants, steroids, and mast cell stabilizers.

### (9) Cells

Exemplary cells suitable for use in the polymer solution include, but are not limited to, cells derived from animals, plants, bacteria, fungi, archaea, viruses, and viroids, and a combination thereof.

### (10) Diagnostic Agents

Optionally, the active agents in the polymer solution are diagnostic agents for imaging or otherwise assessing the tissue of interest. Exemplary diagnostic agents include, but are not limited to, paramagnetic molecules, fluorescent compounds, magnetic molecules, and radionuclides, x-ray imaging agents, MRI imaging agents, ultrasound imaging agents, and contrast media.

ii. Form of Active Agents

The active agents suitable for use in the polymer solution may be present in their neutral form, or in the form of a pharmaceutically acceptable salt (together also referred to herein as “active agents”). In some cases, it may be desirable to prepare a formulation containing a salt of an active agent due to one or more of the salt's advantageous physical properties, such as enhanced stability or a desirable solubility or dissolution profile.

Optionally, the active agent(s) is associated with a carrier, such as particles. The active agent(s) can be complexed with the carrier, covalently attached to the carrier, or encapsulated in the carrier. Exemplary particles that can be used to carry the active agent(s) include, but are not limited to, polymeric particles, lipid particles, liposomes, and dendrimers.

iii. Additives

One or more active agents can be included in the polymer solution to form the fiber construct. Examples of suitable additives for use in the polymer solution include surface modifying agents, thickeners, rheology modifying agents, metal nanofibers/nanotubes (e.g. carbon nanotubes), graphene, and a combination thereof.

Addition of surface modifying agents and/or thickeners in the polymer solution can alter the solution viscosity and charge capacity, and hence alter the fiber physical and mechanical properties. Exemplary surface modifying agents and/or thickeners are gums, such as guar, carageenen, welan gum, xanthan gum, and starch ether. Optionally, the surface modifying agents can also chemically modify the surface of the jet by blooming to the surface of the polymeric fiber in the construct. Optionally, the viscosifying agents can also slowly elute from the surface, thereby modulating cellular behavior on the surface and also conferring a time-dependent mechanical property to the fiber in shorter time scale (e.g. 0-7 days, greater than 7 days, such as 14 days or greater than 14 days following formation of the construct).

Exemplary surface modifying agents include amphiphilic macromers, hydrophilic macromers, and hydrophobic macromers, such as PU backbone with PEG side groups and tecophilic PU, and PU backbone with fluorinated side groups. Exemplary viscosifying agents include macromers such as F-127, hydroxypropyl methylcellulose (HPMC), d,l PLA-PEG copolymers, and tecophilic PU.

Optionally metal nanofibers and/or nanotubes are included in the polymer solution in a sufficient amount to reinforce the mechanical properties of the formed polymeric fibers in the electrospun fiber constructs. Exemplary metal nanofibers and nanotubes include chopped fibers of metals (e.g. magnesium), metal oxide (e.g. Fe₂O₃), and glass and carbon nanotubes.

iv. Concentration of Active Agents and/or Additives

Generally, the concentration of the active agent (or additive) or the total concentration of two or more active agents (or two or more additives) is varied such that a desired amount of the active agent(s) (or additive(s)) is incorporated in the formed fiber construct. The specific amount of the active agent(s) required will vary from subject to subject according to their need and specific use. The specific amount of the additive(s) required will vary depending on the desired property of the polymer solution. The term “total concentration of two or more active agents” refers to the total weight of the active agents relative to the volume of the polymer solution. The term “total concentration of two or more additives” refers to the total weight of the additives relative to the volume of the polymer solution.

Optionally, the concentration of the active agent or the total concentration of two or more active agents is in a range from about from about 0.01% to about 20% (w/v), from about 0.01% to about 15% (w/v), from about 0.01% to about 10% (w/v), from about 0.01% to about 5% (w/v), from about 0.01% to about 1% (w/v), from about 0.01% to about 0.5% (w/v), from about 0.01% to about 0.1% (w/v), from about 0.1% to about 20% (w/v), from about 0.1% to about 15% (w/v), from about 0.1% to about 10% (w/v), from about 0.1% to about 5% (w/v), from about 0.1% to about 1% (w/v), from about 0.1% to about 0.5% (w/v), from about 0.5% to about 20% (w/v), from about 0.5% to about 15% (w/v), from about 0.5% to about 10% (w/v), from about 0.5% to about 5% (w/v), from about 0.5% to about 1% (w/v), from about 1% to about 20% (w/v), from about 1% to about 15% (w/v), from about 1% to about 10% (w/v), or from about 1% to about 5% (w/v) of the polymer solution.

When the polymer solution contains two or more active agents, the concentration of each active agent can be in a suitable range to provide a total concentration of the two or more active agents of from 0.01% to about 20% (w/v). For example, the concentration of each active agent in the polymer solution is in a range from about 0.01% to about 19.99% (w/v), from about 0.02% to about 19.98% (w/v), from about 0.05% to about 19.95% (w/v), from about 0.1% to about 19.9% (w/v), from about 0.5% to about 19.5% (w/v), from about 1% to about 19% (w/v), from about 2% to about 18% (w/v), from about 5% to about 15% (w/v), from about 0.01% to about 10% (w/v), from about 0.01% to about 5% (w/v), from about 0.01% to about 2% (w/v), from about 0.01% to about 1% (w/v), from about 0.05% to about 5% (w/v), from about 0.05% to about 2% (w/v), from about 0.05% to about 1% (w/v), from about 0.1% to about 10% (w/v), from about 0.1% to about 5% (w/v), or from about 0.1% to about 1% (w/v) of the polymer solution.

Optionally, the concentration of the additive or the total concentration of two or more additives is in a range from about from about 0.01% to about 20% (w/v), from about 0.01% to about 15% (w/v), from about 0.01% to about 10% (w/v), from about 0.01% to about 5% (w/v), from about 0.01% to about 1% (w/v), from about 0.01% to about 0.5% (w/v), from about 0.01% to about 0.1% (w/v), from about 0.1% to about 20% (w/v), from about 0.1% to about 15% (w/v), from about 0.1% to about 10% (w/v), from about 0.1% to about 5% (w/v), from about 0.1% to about 1% (w/v), from about 0.1% to about 0.5% (w/v), from about 0.5% to about 20% (w/v), from about 0.5% to about 15% (w/v), from about 0.5% to about 10% (w/v), from about 0.5% to about 5% (w/v), from about 0.5% to about 1% (w/v), from about 1% to about 20% (w/v), from about 1% to about 15% (w/v), from about 1% to about 10% (w/v), or from about 1% to about 5% (w/v) of the polymer solution.

When the polymer solution contains two or more additives, the concentration of each additive can be in a suitable range to provide a total concentration of the two or more additives of from 0.01% to about 20% (w/v). For example, the concentration of each additive in the polymer solution is in a range from about 0.01% to about 19.99% (w/v), from about 0.02% to about 19.98% (w/v), from about 0.05% to about 19.95% (w/v), from about 0.1% to about 19.9% (w/v), from about 0.5% to about 19.5% (w/v), from about 1% to about 19% (w/v), from about 2% to about 18% (w/v), from about 5% to about 15% (w/v), from about 0.01% to about 10% (w/v), from about 0.01% to about 5% (w/v), from about 0.01% to about 2% (w/v), from about 0.01% to about 1% (w/v), from about 0.05% to about 5% (w/v), from about 0.05% to about 2% (w/v), from about 0.05% to about 1% (w/v), from about 0.1% to about 10% (w/v), from about 0.1% to about 5% (w/v), or from about 0.1% to about 1% (w/v) of the polymer solution.

d. Polymer Concentrations

The concentration of the polymer or the total concentration of two or more polymers in the polymer solution can be varied to achieve a desired solution viscosity, surface tension of polymer solution, solution conductivity, and/or polymer jet velocity, each of which can affect the morphology and diameter of the polymer fiber being formed. The term “total concentration of two or more polymers” refers to the total weight of the polymers in the polymer solution relative to the volume of the polymer solution.

Optionally, the concentration of the polymer or the total concentration of two or more polymers is in a range from about 1% to about 90% (w/v), from about 1% to about 85% (w/v), from about 1% to about 80% (w/v), from about 1% to about 75% (w/v), from about 1% to about 70% (w/v), from about 1% to about 65% (w/v), from about 1% to about 60% (w/v), from about 1% to about 55% (w/v), from about 1% to about 50% (w/v), from about 1% to about 45% (w/v), from about 1% to about 40% (w/v), from about 1% to about 35% (w/v), from about 1% to about 30% (w/v), from about 1% to about 25% (w/v), from about 1% to about 20% (w/v), from about 1% to about 15% (w/v), or from about 1% to about 10% (w/v), such as about 7.5% (w/v) of the polymer solution.

When the polymer solution contains two or more polymers, the concentration of each polymer can be in a suitable range to provide a total concentration of the two or more polymers of from about 1% to about 90% (w/v). For example, the concentration of each polymer is in a range from about 0.1% to about 89.9% (w/v), from about 0.5% to about 89.5% (w/v), from about 1% to about 89% (w/v), from about 5% to about 85% (w/v), from about 0.1% to about 10% (w/v), from about 0.1% to about 20% (w/v), from about 0.1% to about 50% (w/v), from about 1% to about 85% (w/v), from about 1% to about 50% (w/v), from about 1% to about 30% (w/v), from about 1% to about 20% (w/v), from about 0.1% to about 15% (w/v), from about 1% to about 15% (w/v), from about 0.5% to about 50% (w/v), from about 0.5% to about 20% (w/v), or from about 0.5% to about 15% (w/v).

e. Polymer Hydrodynamic Radius

The polymer can have different hydrodynamic radius/chain conformation depending on the selection of the solvent. For example, a 20% (w/v) Tecoflex® in THF has a chain confirmation that is different from a 20% (w/v) Tecoflex® in dimethylacetamide (DMAC). The hydrodynamic radius of the polymer can be varied to modify the properties of formed polymer fiber, such as the polymer fiber diameter.

### 3. Extrusion Rate

Following applying an electrostatic charge to the ejection device or a component thereof using a power supply, the polymer solution is extruded out of the ejection device via the ejector by an electrostatic force at an extrusion rate. Generally, the polymer solution is extruded at an extrusion rate in a range from about 0.01 mL/h to about 50 mL/h.

Optionally, the polymer solution is extruded at an extrusion rate in a range from about 0.01 mL/h to about 48 mL/h, from about 0.01 mL/h to about 45 mL/h, from about 0.01 mL/h to about 40 mL/h, from about 0.01 mL/h to about 35 mL/h, from about 0.01 mL/h to about 30 mL/h, from about 0.01 mL/h to about 25 mL/h, from about 0.01 mL/h to about 20 mL/h, from about 0.01 mL/h to about 15 mL/h, from about 0.01 mL/h to about 10 mL/h, from about 0.01 mL/h to about 5 mL/h, from about 0.05 mL/h to about 20 mL/h, from about 0.05 mL/h to about 48 mL/h, from about 0.05 mL/h to about 45 mL/h, from about 0.05 mL/h to about 40 mL/h, from about 0.05 mL/h to about 35 mL/h, from about 0.05 mL/h to about 30 mL/h, from about 0.05 mL/h to about 25 mL/h, from about 0.05 mL/h to about 20 mL/h, from about 0.05 mL/h to about 15 mL/h, from about 0.05 mL/h to about 10 mL/h, from about 0.1 mL/h to about 48 mL/h, from about 0.1 mL/h to about 45 mL/h, from about 0.1 mL/h to about 40 mL/h, from about 0.1 mL/h to about 35 mL/h, from about 0.1 mL/h to about 30 mL/h, from about 0.1 mL/h to about 25 mL/h, from about 0.1 mL/h to about 20 mL/h, from about 0.1 mL/h to about 15 mL/h, from about 0.1 mL/h to about 10 mL/h, from about 0.1 mL/h to about 5 mL/h, from about 1 mL/h to about 48 mL/h, from about 1 mL/h to about 45 mL/h, from about 1 mL/h to about 40 mL/h, from about 1 mL/h to about 35 mL/h, from about 1 mL/h to about 30 mL/h, from about 1 mL/h to about 25 mL/h, from about 1 mL/h to about 20 mL/h, from about 1 mL/h to about 15 mL/h, from about 1 mL/h to about 10 mL/h, from about 1 mL/h to about 5 mL/h, such as about 2.5 mL/h.

### B. Selecting a Collector

The method can include a step of selecting a collector prior to applying an electrostatic charge to the ejection device or a component thereof.

A user can select a collector such that the pattern on the collection surface is sufficient to generate the fiber construct having a desired grammage. For example, the collector is selected such that the pattern on the collection surface is sufficient to generate the fiber construct having a grammage that is at least 5% higher than a grammage of a fiber construct formed on an un-patterned collector under the same conditions.

By selecting collector with a certain pattern on the collection surface, the direction and/or trajectory of the electrostatic field generated between the ejector and the collector can be manipulated or changed. Accordingly, the properties of the fiber construct, such as the grammage of the fiber construct can be manipulated.

Without being bound to theories, the grammage of the fiber construct can be influenced by the imposed electrostatic field between the ejector and the grounded collector. A driver of fiber deposition is the magnitude and direction of the electrical field generated between the syringe needle and the target (Angammana and Jayaram, IEEE Transactions on Industry Applications 2012, 48, 808-815; Kong, et al., Journal of Materials Science 2007, 42, 8106-8112). When the same electric field magnitude is used, any influence of the electric field on fiber construct properties can be varied by varying direction or trajectory of the electrostatic field between the ejector and the grounded collector. The direction and/or trajectory of the electrostatic field can be manipulated or changed by varying the pattern on the collection surface of the collector.

For example, in the lines pattern, the electrostatic field forms clear and distinct organized field lines and vectors between the ejector and grounded collector, with small variations near the collector. The zigzag pattern increases distortion of the electric field, with fringe effects near the edges of the zigzag pattern of the collector occur, influencing polymer jet trajectory, resulting in less overall deposition over the copper trace patterns.

### C. Optional Steps

In addition to the steps described above, which include: step (i) applying an electrostatic charge on the ejection device or a component thereof, and optionally (ia) selecting a collector prior to step (i), the method may include one or more additional steps. The additional steps can occur prior to, simultaneous with, or subsequent to step (ia) but prior to step (i).

1. Loading a Reservoir with the Polymer Solution

The method may include a step of loading the polymer solution in the reservoir of the ejection device. For example, a reservoir, such as a syringe, is filled with the polymer solution or the reservoir is pre-filled with the polymer solution. Typically, following the loading step, the polymer solution in the reservoir does not contain air bubbles. Techniques for filling a reservoir, such as a syringe, without air bubbles are known in the art.

The method may further include a step of mixing a polymer, optionally more than one polymer, with a solvent to form the polymer solution prior to loading the reservoir.

The particular solvent can be selected based on the solubility of the polymer used to form the fiber construct such that the polymer is dissolved in the solvent.

Optionally, the solvent is selected based on the solubility of the polymer and an active agent and/or additive. The polymer and active agent and/or additive can be mixed to form the polymer solution.

In some embodiments, a second reservoir, such as a second syringe, that is different from the reservoir containing the polymer solution is filled with or contains an active agent solution or suspension. Optionally, the method includes a further step of mixing an active agent, optionally more than one active agent, with a solvent to form the active agent solution or suspension prior to filling the reservoir. The solvent for the active agent solution or suspension can be selected based on the solubility of the active agent such that the active agent is dissolved or suspended in the solvent.

### 2. Assembling the Electrospinning System

The method may include a step of assembling the electrospinning system to a configuration subsequent to step (ia) and prior to step (i). Generally, the configuration of the assembled electrospinning system is suitable for the extruded polymer solution to reach the collection surface of the collector.

The assembled electrospinning system can have any of the configuration described above. For example, the collector is located at an angle (θ) in a range from 0° to 80°, from 0° to 60°, from 0° to 40°, from 0° to 20°, or from 0° to 10°, such as about 0° between a horizontal axis of the collector and an imaginary longitudinal line running through the center of the tip of the ejector and at a distance from the ejection device in a range from about 5 cm to about 100 cm, 5 cm to about 80 cm, 5 cm to about 50 cm, from about 5 cm to about 25 cm, or from about 10 cm to about 20 cm, such as about 15 cm.

### 3. Applying Pressure/Vacuum

The method may include a step of applying pressure/vacuum to the reservoir to deliver the polymer solution to the ejector. The pressure and/or vacuum can be applied simultaneous with or subsequent to applying the electrostatic charge to the ejection device or a component thereof.

For example, a pressurization component, such as a pressurized air tank and/or a vacuum pump, is connected to the reservoir. A user, such as a human, can manually operate the air tank or the vacuum pump to apply and/or adjust pressure and/or vacuum to the syringe containing the polymer solution such that the polymer solution is pushed to the tip portion of the ejector.

Optionally, the pressurization component can be adjusted by a programmed processor in response to data from one or more sensors to maintain a predetermined pressure and/or vacuum or to modify the pressure and/or vacuum in the reservoir.

4. Repeating Step (Ia) and/or Step (i)

The method may include repeating step (i) applying an electrostatic charge to the ejector or a component thereof to generate fiber construct of multiple layers, i.e. at least two layers.

Optionally, the method includes repeating the step of selecting a collector and the step of applying an electrostatic charge to multiple fiber constructs of different properties and shape.

For example, a user selects a first collector containing a first pattern. Following formation of a first fiber construct on the first pattern, the user selects a second collector containing a second patter. Following formation of a second fiber construct on the second pattern, the user attached the first and second fiber constructs to form a fiber construct of a desired 3D shape.

### 5. Adjusting Temperature of Reservoir

The method may include the step of adjusting the temperature of the reservoir and/or the ejector prior to or simultaneously with applying the electrostatic charge to the ejector or a component thereof. Varying the temperature of the reservoir and/or the ejector can vary the temperature of the polymer solution and hence change the conductivity and viscosity of the polymer solution.

A user, such as a human, may adjust the temperature of the reservoir and/or the ejector and hence the polymer solution via a temperature control component such that the polymer solution can be extruded from the ejector and form a jet towards the collector.

For example, the user can manually active a heating component wrapped around the reservoir and/or the ejector to apply heat to the polymer solution contained within. Alternatively, the user can manually active a cooling component wrapped around the reservoir and/or the ejector to lower the temperature of the polymer solution contained within. The heating and/or cooling components can be any of those described above.

Optionally, the temperature control component can be adjusted by a programmed processor in response to data from one or more sensors to maintain a predetermined temperature or to modify the temperature of the polymer solution.

### 6. Post-Processing of Fiber Constructs

The method may include one or more post-processing steps subsequent to step (i).

Optionally, in a post-processing step, the formed fiber construct is shaped into a desired shape. Techniques for shaping a formed fiber construct are known in the art, such as laser-based processing and/or nanoimprinting lithography.

Optionally, in a post-processing step, the formed fiber construct is cut into a desired length. The length of the fiber construct after cutting depends on the specific use of the fiber construct. For example, after cutting, the fiber construct can have a length in a range from about 10 nm to about 10 cm, from about 100 nm to about 10 cm, from about 10 nm to about 1 cm, from about 1 μm to about 10 cm, from about 1 μm to about 1 cm, from about 10 μm to about 10 cm, or from about 10 μm to about 1 cm. Techniques for cutting a formed fiber construct are known in the art, such as laser cutting.

Optionally, in the post-processing step, the user triggers cross-linking in the formed fiber construct using ultraviolet irradiation or temperature. For example, the fiber construct is formed from polyglycerol sebacate and the user triggers cross-linking of the secondary hydroxyl groups on the glycerol of the formed fiber construct via ultraviolet irradiation and/or heating the fiber construct.

Optionally, in the post-processing step, the user deposits a coating material on the surface of the formed fiber construct. Techniques for coating a formed fiber construct are known in the art, such as physical vapor deposition and chemical vapor deposition. Examples of materials suitable for coating the formed fiber construct include, but are not limited to, NiTi, carbon nanotubes, metals (e.g. gold, silver, etc.), pyrolitic C, metal oxides (e.g. Ir oxide). Coating the formed fiber construct using the above-described materials achieve surface mineralization or metallization, which provides a load bearing property and/or alter the surface property of the fiber construct.

Optionally, in the post-processing step, the user heats the formed fiber construct at a temperature that is the midpoint between Tg and Tm to induce crystallinity in the fiber construct. For example, the fiber construct is formed from polylactic acid and the user heats the fiber construct at midpoint between Tg and Tm (such as for example, ˜ 110° C.) to induce crystallinity and thus fiber strength. Heat can be applied from the surface of the electrospun fiber construct. This can create a thermal gradient, resulting in a distribution of crystallinity in the fibers, i.e. the inner fibers are less crystalline than the outer fibers.

Optionally the method includes one or more of the following post-processing steps: crosslining the fibers in the formed fiber construct, shaping the formed fiber construct, cutting the formed fiber construct to a desired length, depositing a coating material on the surface of the formed fiber construct, and/or heating the formed fiber construct.

7. Adding Active Agents and/or Additives

The method may include a step of adding one or more active agents and/or one or more additives in the polymer solution and/or onto/into the formed polymer construct. The active agent(s) and/or additive(s) can be added into the polymer solution prior to applying the electrostatic charge to the ejection device or a component thereof. The active agent and/or additive can dissolve in the polymer solution or suspended in the polymer solution.

When two or more active agents or additives are added into the polymer solution, all of the active agents or additives can dissolve or be suspended in the polymer solution. Alternatively, one or more of the active agents or additive can dissolve in the polymer solution and one or more active agent or additive can be suspended in the polymer solution.

Alternatively or additionally, the active agent(s) and/or additive(s) can be added onto and/or into the formed fiber construct. For example, an active agent and/or an additive can be coated onto the formed fiber construct and/or dispersed inside the formed fiber construct.

Any of the steps described above for the method of generating electrospun fiber construct may be modified to a generate fiber construct of desired dimensions for a desired purpose.

### III. Electrospun Fiber Constructs

### 1. Components of Fiber Construct

The formed electrospun fiber construct contains polymeric fibers formed by the biocompatible polymer solution ejected onto the collection surface of the collector. Optionally, the formed fiber construct also contains one or more active agent(s) and/or one or more additive(s).

a. Polymers

The fiber construct can contain polymeric fibers formed by any one of the biocompatible polymers described above. Optionally, the fiber construct contains polymeric fibers formed by a combination of the biocompatible polymers described above. For example, the fiber construct contains polymeric fibers formed by a natural polymer described above, a synthetic polymer described above, or a combination thereof.

The biocompatible polymers forming the polymeric fibers of the fiber construct can be biodegradable or non-biodegradable. Optionally, the polymeric fibers of the fiber construct are formed from one or more biodegradable polymers described above. Optionally, the polymeric fibers of the fiber construct are formed from one or more non-biodegradable polymers described above. Optionally, the polymeric fibers of the fiber construct are formed from one or more biodegradable polymers and one or more non-biodegradable polymers described above.

When the fiber construct are formed by two or more polymers, the weight percentage of each polymer in the fiber construct can be in a suitable range from about 0.1% to about 90%, from about 0.1% to about 89.9%, from about 0.5% to about 89.5%, from about 1% to about 89%, from about 5% to about 85%, from about 0.1% to about 10%, from about 0.1% to about 20%, from about 0.1% to about 50%, from about 1% to about 85%, from about 1% to about 50%, from about 1% to about 30%, from about 1% to about 20%, from about 0.1% to about 15%, from about 1% to about 15%, from about 0.5% to about 50%, from about 0.5% to about 20%, or from about 0.5% to about 15%.

Optionally, the fiber construct are formed by two different polymers. The weight ratio between the first and second polymer can be in a range from about 0.01 to about 1000, from about 0.01 to about 500, from about 0.01 to about 200, from about 0.01 to about 150, from about 0.01 to about 100, from about 0.01 to about 50, from about 0.01 to about 20, from about 0.01 to about 10, from about 0.05 to about 500, from about 0.02 to about 200, from about 0.05 to about 150, from about 0.05 to about 100, from about 0.05 to about 50, from about 0.02 to about 20, from about 0.02 to about 10, from about 0.1 to about 1000, from about 0.1 to about 500, from about 0.1 to about 250, from about 0.1 to about 100, from about 0.1 to about 50, from about 0.1 to about 20, or from about 0.1 to about 10.

b. Active Agent(s) and/or Additive(s)

The formed fiber construct can also contain one or more active agent(s) and/or one or more additive(s) described above. The active agent and/or additive can be coated on the surface of the fiber construct or embedded in the fiber construct, or a combination thereof. For example, one or more active agent(s) is coated on the surface of the fiber construct and one or more active agent(s) is embedded in the fiber construct. Optionally, one or more additive(s) is coated on the surface of the fiber construct and one or more additive(s) is embedded in the fiber construct.

When the fiber construct contains both active agent(s) and additive(s), they can be independently coated on the surface of the fiber construct or embedded in the fiber construct. For example, when the fiber construct contains both active agent(s) and additive(s), one or more active agent(s) is coated on the surface of and/or embedded in the fiber construct, and one or more additive(s) is coated on the surface of and/or embedded in the fiber construct.

The fiber construct may contain one or more active agent(s) associated with a carrier or a combination of one or more active agent(s) in free form and one or more active agent(s) associated with a carrier. Optionally, the active agent(s) or at least one active agent of two or more active agents of the fiber construct can be associated with a carrier.

For example, the active agent(s) can be complexed with the carrier, covalently attached to the carrier, or encapsulated in the carrier. The carrier containing the active agent(s) is coated on the surface of the fiber construct or embedded in the fiber construct.

### 2. Fiber Construct Parameters

The fiber construct can be characterized by physical and mechanical parameters described below. In particular, the fiber construct is characterized by its grammage and open area. The term “open area” refers to the total area of the open portions of the construct that are located between the polymeric fibers. Generally, the open area of the fiber construct corresponds to the total area of a target that is not covered by polymeric fibers after a fiber construct is formed thereon.

The microporosity and macroporosity of the fiber construct can be controlled by using collectors with different patterns. The microporosity can be described by parameters such as the grammage of the fiber construct. The macroporosity can be described by parameters such as the open area of the fiber construct. The grammage and/or open area of the fiber construct can be controlled by using collectors with different patterns. Typically, the pattern of the collector is effective to form fiber constructs of a desired grammage and/or residual open area.

In some embodiments, an electrospun fiber construct containing polymeric fibers and open spaces has a microporosity and a macroporosity, where the electrospun fiber construct has a smaller microporosity and/or a greater macroporosity than a control electrospun fiber construct formed under the same conditions and using an un-patterned collector having the same total area.

In some embodiments, the electrospun fiber construct has a microporosity and/or macroporosity such that when seeded with a type of cells and exposed to cell growth conditions, the adhesion of the cells to the electrospun fiber construct is greater than the adhesion of the same type of cells under the same cell growth conditions to the control electrospun fiber construct.

a. Grammage

Typically, the electrospun fiber constructs formed using the electrospinning system described herein has a grammage that is at least 5% higher than a grammage of a fiber construct formed on an un-patterned collector under the same conditions. For example, the formed electrospun fiber constructs using the electrospinning system described herein has a grammage that is at least 6% higher, at least 8% higher, at least 10% higher, at least 12% higher, at least 15% higher, at least 18% higher, at least 20% higher, up to 80% higher, up to 75% higher, up to 70% higher, up to 65% higher, up to 60% higher, up to 55% higher, up to 50% higher, in a range from about 5% to about 80% higher, from about 5% to about 75% higher, from about 5% to about 70% higher, from about 5% to about 65% higher, from about 5% to about 60% higher, from about 5% to about 50% higher, from about 10% to about 80% higher, from about 10% to about 75% higher, from about 10% to about 70% higher, from about 10% to about 65% higher, from about 10% to about 60% higher, from about 10% to about 50% higher, from about 15% to about 80% higher, from about 15% to about 75% higher, from about 15% to about 70% higher, from about 15% to about 65% higher, from about 15% to about 60% higher, from about 15% to about 50% higher, from about 20% to about 80% higher, from about 20% to about 75% higher, from about 20% to about 70% higher, from about 20% to about 65% higher, from about 20% to about 60% higher, or from about 20% to about 50% higher than a grammage of a fiber construct formed on an un-patterned collector under the same conditions.

Optionally, the formed electrospun fiber constructs using the electrospinning system described herein has a grammage that is at least 10% higher, at least 15% higher, at least 40% higher, from about 5% to about 80% higher, from about 10% to about 80% higher, from about 15% to about 80% higher, from about 20% to about 80% higher than a grammage of a fiber construct formed on an un-patterned collector under the same conditions. For example, the formed electrospun fiber constructs using the electrospinning system described herein has a grammage that is about 40% or about 45% higher than a grammage of a fiber construct formed on an un-patterned collector under the same conditions.

Optionally, the fiber construct has a grammage in a range from about 18 μg/mm² to about 100 μg/mm². For example, the fiber construct has a grammage in a range from about 19 μg/mm² to about 100 μg/mm², from about 20 μg/mm² to about 100 μg/mm², from about 21 μg/mm² to about 100 μg/mm², from about 22 μg/mm² to about 100 μg/mm², from about 23 μg/mm² to about 100 μg/mm², from about 24 μg/mm² to about 100 μg/mm², from about 18 μg/mm² to about 95 μg/mm², from about 18 μg/mm² to about 90 μg/mm², from about 18 μg/mm² to about 85 μg/mm², from about 18 μg/mm² to about 80 μg/mm², from about 18 μg/mm² to about 75 μg/mm², from about 18 μg/mm² to about 70 μg/mm², from about 18 μg/mm² to about 65 μg/mm², from about 18 μg/mm² to about 60 μg/mm², from about 18 μg/mm² to about 55 μg/mm², from about 18 μg/mm² to about 50 μg/mm², from about 18 μg/mm² to about 45 μg/mm², from about 18 μg/mm² to about 40 μg/mm², from about 18 μg/mm² to about 35 μg/mm², from about 18 μg/mm² to about 30 μg/mm², such as about 18 μg/mm², about 19 μg/mm², 20 μg/mm², about 22 μg/mm², or about 25 μg/mm².

b. Open Area

Typically, the electrospun fiber constructs formed using the electrospinning system described herein has an open area. Typically, the formed fiber construct has an open area in a range from about 1% to about 99% of the total of the fiber area and the open area. The term “fiber area” refers to the total of fiber covered-portions of the fiber construct. Generally, the fiber area of a fiber construct corresponds to the total area of a target that is covered by polymeric fibers after fiber construct is formed on the collection surface of the collector.

For example, the formed fiber construct has an open area in a range from about 1% to about 98%, from about 1% to about 95%, from about 1% to about 90%, from about 1% to about 85%, from about 1% to about 80%, from about 1% to about 75%, from about 1% to about 70%, from about 1% to about 65%, from about 1% to about 60%, from about 1% to about 55%, from about 1% to about 50%, from about 1% to about 45%, from about 1% to about 40%, from about 1% to about 35%, from about 1% to about 30%, from about 5% to about 98%, from about 5% to about 95%, from about 5% to about 90%, from about 5% to about 85%, from about 5% to about 80%, from about 5% to about 75%, from about 5% to about 70%, from about 5% to about 65%, from about 5% to about 60%, from about 5% to about 55%, from about 5% to about 50%, from about 5% to about 45%, from about 5% to about 40%, from about 5% to about 35%, from about 5% to about 30%, from about 8% to about 98%, from about 8% to about 95%, from about 8% to about 90%, from about 8% to about 85%, from about 8% to about 80%, from about 8% to about 75%, from about 8% to about 70%, from about 8% to about 65%, from about 8% to about 60%, from about 8% to about 55%, from about 8% to about 50%, from about 8% to about 45%, from about 8% to about 40%, from about 8% to about 35%, from about 8% to about 30%, from about 10% to about 98%, from about 10% to about 95%, from about 10% to about 90%, from about 10% to about 85%, from about 10% to about 80%, from about 10% to about 75%, from about 10% to about 70%, from about 10% to about 65%, from about 10% to about 60%, from about 10% to about 55%, from about 10% to about 50%, from about 10% to about 45%, from about 10% to about 40%, from about 10% to about 35%, from about 10% to about 30%, from about 15% to about 98%, from about 15% to about 95%, from about 15% to about 90%, from about 15% to about 85%, from about 15% to about 80%, from about 15% to about 75%, from about 15% to about 70%, from about 15% to about 65%, from about 15% to about 60%, from about 15% to about 55%, from about 15% to about 50%, from about 15% to about 45%, from about 15% to about 40%, from about 15% to about 35%, from about 15% to about 30%, from about 20% to about 98%, from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 20% to about 55%, from about 20% to about 50%, from about 20% to about 45%, from about 20% to about 40%, from about 20% to about 35%, from about 20% to about 30%, from about 25% to about 98%, from about 25% to about 95%, from about 25% to about 90%, from about 25% to about 85%, from about 25% to about 80%, from about 25% to about 75%, from about 25% to about 70%, from about 25% to about 65%, from about 25% to about 60%, from about 25% to about 55%, from about 25% to about 50%, from about 25% to about 45%, from about 25% to about 40%, or from about 25% to about 35% of the total of the fiber area and the open area.

Optionally, the formed fiber construct has an open area in a range from about 8% to about 90%, from about 10% to about 90%, from about 15% to about 90%, from about 20% to about 90%, from about 25% to about 90%, from about 8% to about 70%, from about 10% to about 70%, from about 15% to about 70%, from about 20% to about 70%, from about 25% to about 70%, from about 8% to about 50%, from about 10% to about 50%, from about 15% to about 50%, from about 20% to about 50%, from about 25% to about 50%, from about 8% to about 40%, from about 10% to about 40%, from about 15% to about 40%, from about 20% to about 40%, or from about 25% to about 40% of the total of the fiber area and the open area.

c. Fiber Diameters

The fiber construct typically contains polymeric fibers having the same or similar diameter. Generally, the diameter of the polymeric fibers is in a range from about 10 nm to about 10 μm. For example, the diameter of the polymer fiber is in a range from about 20 nm to about 10 μm, from about 30 nm to about 10 μm, from about 40 nm to about 10 μm, from about 50 nm to about 10 μm, from about 60 nm to about 10 μm, from about 70 nm to about 10 μm, from about 80 nm to about 10 μm, from about 90 nm to about 10 μm, from about 0.1 μm to about 10 μm, from about 0.1 μm to about 9 μm, from about 0.1 μm to about 8 μm, from about 0.1 μm to about 7 μm, from about 0.1 μm to about 6 μm, from about 0.1 μm to about 5 μm, from about 0.5 μm to about 10 μm, from about 0.5 μm to about 9 μm, from about 0.5 μm to about 8 μm, from about 0.5 μm to about 7 μm, from about 0.5 μm to about 6 μm, from about 0.5 μm to about 5 μm, from about 1 μm to about 10 μm, from about 1 μm to about 9 μm, from about 1 μm to about 8 μm, from about 1 μm to about 7 μm, from about 1 μm to about 6 μm, or from about 1 μm to about 5 μm, such as about 3.5 μm or about 4 μm.

Optionally, the fiber construct contains polymeric fibers of two or more different diameters. When the polymeric fibers of the fiber construct have two or more different diameters, each diameter can be in the ranges described above.

The diameter of the formed polymeric fibers can be modified by varying the operation parameters of the method for forming the fiber construct, such as the applied electrostatic charge, the extrusion rate of the polymer solution, the polymer concentration, the distance between the ejection device and the collector, temperature and humidity, etc. These operation parameters can be varied to achieve a desired polymer fiber diameter in the fiber construct.

d. Other Parameters of Fiber Construct

In addition to the parameters described above, the formed fiber construct can be characterized by other parameters, such as fiber excess and contact angle.

i. Fiber Excess

The fiber construct may be characterized by fiber excess or normalized fiber excess. The term “fiber excess” refers to the total spillover areas of the polymeric fibers that exceed the boundaries of each conductive trace on the collection surface. The fiber excess may be calculated by subtracting the conductive trace area from the fiber area. The term “normalized fiber excess” refers to the amount of fiber excess relative to the conductive trace area. This is an index of accuracy of the polymeric fibers distributing onto the collector.

Optionally, the normalized fiber excess of the fiber construct is up to 92%, up to 85%, up to about 81%, up to about 80%, up to about 75%, in a range from about 5% to about 95%, from about 10% to about 95%, from about 15% to about 95%, from about 20% to about 95%, from about 10% to about 90%, from about 10% to about 85%, from about 10% to about 80%, from about 10% to about 75%, from about 20% to about 85%, from about 20% to about 80%, from about 20% to about 75%, or from about 30% to about 75%, such as about 70%, about 80%, about 85%, or about 92%.

Without being bound by theory, as the conductive trace area decreases, the non-conductive area increases and the fiber excess generated on a given conductive trace increases, extending beyond the trace boundary (Pan, et al., Journal of Macromolecular Science, Part B 2008, 47, 735-742). A related inverse relationship may exist in terms of grammage: as the conductive trace area decreases, more dense fiber bundles are deposited on more sparse targets, i.e. increased grammage of formed fiber construct (Vaquette and Cooper-White, Acta Biomaterialia 2011, 7, 2544-2557).

ii. Contact Angle

The hydrophilicity or hydrophobicity of the fiber construct may be characterized by contact angle. The contact angle of a droplet on the surface of the fiber construct represents the hydrophilicity or hydrophobicity of the fiber construct. The hydrophilicity of the fiber construct surface can be adjusted by adjusting the hydrophilicity of the polymer forming the fibers and the grammage of the fiber construct.

Techniques of measuring contact angles of a material are known. For example, the static sessile drop angle of deionized water on the surface of the fiber construct using an optional contact angle meter. A specific example is described in the Examples. Generally, if the water contact angel is smaller than about 90, the surface of the fiber construct is considered hydrophilic. If the water contact angel is larger than 90, the surface of the fiber construct is considered hydrophobic.

Optionally, the water contact angle on the surface of the fiber construct is in a range from about 0° to about 150°, from about 0° to about 90°, or from about 90° to about 150°.

### IV. Compositions Containing Electrospun Fiber Constructs

The electrospun fiber constructs may be used on their own or may be combined with active agents, excipients, and/or carriers to form compositions.

The electrospun fiber constructs may be used for forming engineered tissues or implants, wound dressings, drug delivery formulations, and implant or device coatings. In these embodiments, the electrospun fiber constructs may include active agents, excipients, and/or carriers suitable for particular uses.

### A. Engineered Tissue or Implant

Optionally, the electrospun fiber construct is used for forming an engineered tissue or implant. In these embodiments, the electrospun fiber construct contains polymeric fibers and optionally one or more biological cell(s) and/or one or more active agent(s).

The engineered tissue or implant formed by an electrospun fiber construct or two or more electrospun fiber constructs can be used for the construction of a tissue or an organ or a portion of an organ in vivo or in vitro.

For example, the electrospun fiber construct is used as a template or scaffold for growing cells to form a tissue or organ in vivo or in vitro. For example, the engineered tissue or implant formed by one or more electrospun fiber construct is placed partially or wholly within a patient's body for one or more therapeutic or prophylactic purposes, such as for tissue augmentation, contouring, restoring physiological function, repairing or restoring tissues damaged by a disease or trauma, and/or delivering therapeutic agents to normal, damaged, or diseased organs or tissue.

### 1. Exemplary Shapes

Optionally, the electrospun fiber construct for forming an engineered tissue or implant has a shape suitable to fit a defect or site of an organ or tissue of a subject, such as a mammal. For example, the engineered tissue or implant formed by the fiber construct has a 2-D shape, such as a circle, an oval, a rectangular, a square, a triangle, a polygonal, or an irregular shape suitable for fitting a defect or site of an organ or tissue of the subject. For example, the engineered tissue or implant formed by the fiber construct has a 3-D shape, such as a cylinder, a sphere, a cone, a cube, a polyhedron, a cuboid, an ellipsoid, a donut, or an irregular 3-D shape suitable for fitting a defect or site of an organ or tissue of the subject. When the engineered tissue or implant contains two or more electrospun fiber constructs, the electrospun fiber constructs can be sutured, sealed, stapled, or stacked to one another, or a combination thereof, to form a desired 3-D shape, such as those described above.

### 2. Open Area and Grammage

In these embodiments, the electrospun fiber construct has an open area in a range from about 8% to about 90%, from about 10% to about 90%, from about 15% to about 90%, from about 20% to about 90%, from about 25% to about 90%, from about 8% to about 70%, from about 10% to about 70%, from about 15% to about 70%, from about 20% to about 70%, from about 25% to about 70%, from about 8% to about 50%, from about 10% to about 50%, from about 15% to about 50%, from about 20% to about 50%, from about 25% to about 50%, from about 8% to about 40%, from about 10% to about 40%, from about 15% to about 40%, from about 20% to about 40%, or from about 25% to about 40% of the total of the fiber area and the open area.

Generally, the grammage of the fiber construct for forming an engineered tissue or implant is in a range from about 18 μg/mm² to about 100 μg/mm², from about 18 μg/mm² to about 80 μg/mm², 18 μg/mm² to about 60 μg/mm², 18 μg/mm² to about 50 μg/mm², 18 μg/mm² to about 40 μg/mm², 18 μg/mm² to about 30 μg/mm², 18 μg/mm² to about 20 μg/mm².

For example, the fiber construct for forming an engineered tissue or implant has an open area in a range from about 8% to about 90%, from about 10% to about 90%, from about 15% to about 90%, from about 20% to about 90%, from about 25% to about 90%, from about 8% to about 70%, from about 10% to about 70%, from about 15% to about 70%, from about 20% to about 70%, from about 25% to about 70%, from about 8% to about 50%, from about 10% to about 50%, from about 15% to about 50%, from about 20% to about 50%, from about 25% to about 50%, from about 8% to about 40%, from about 10% to about 40%, from about 15% to about 40%, from about 20% to about 40%, or from about 25% to about 40% of the total of the fiber area and the open area and a grammage in a range from about 18 μg/mm² to about 100 μg/mm², from about 18 μg/mm² to about 80 μg/mm², 18 μg/mm² to about 60 μg/mm², 18 μg/mm² to about 50 μg/mm², 18 μg/mm² to about 40 μg/mm², 18 μg/mm² to about 30 μg/mm², 18 μg/mm² to about 20 μg/mm².

Optionally, the electrospun fiber construct(s) for forming an engineered tissue or implant has a grammage and open area suitable for cells to adhere to and retain on the polymer fiber bundles of the fiber construct.

### 3. Exemplary Polymers

Polymers suitable for forming polymeric fibers of the electrospun fiber constructs that form engineered tissues or implants can be any biocompatible polymers described above. Preferably, the electrospun fiber constructs forming the engineered tissues or implants are formed from biodegradable polymers, such as a natural polymer, optionally two or more natural polymers, or a biodegradable synthetic polymer, optionally two or more biodegradable synthetic polymers, or a combination thereof.

Examples of polymers suitable for forming electrospun fiber constructs for use as engineered tissue or implants include natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof, and biodegradable synthetic polymers such as polymers of hydroxyacids such as lactic acid and glycolic acid (e.g. poly(lactic-co-glycolic acid)), and copolymers with PEG, polyanhydrides, poly(ortho)esters, polyurethanes, poly(butyric acid), poly(valeric acid), poly(lactide-coaprolactone), blends and copolymers thereof.

### 4. Exemplary Cells

Optionally, the electrospun fiber construct contains one or more biological cell(s) adhered thereto. The cells are typically entrapped in the fiber area of the electrospun fiber construct. The terms “cells” and “biological cells” are used interchangeably herein.

In these embodiments, the electrospun fiber construct has a microporosity and/or a macroporosity such that when seeded with a type of cells and exposed to cell growth conditions, the adhesion of the cells to the electrospun fiber construct is greater than the adhesion of the same type of cells under the same cell growth conditions to the control electrospun fiber construct.

The biological cells are cultured and grow on an electrospun fiber construct to form a tissue or organ in vivo or in vitro. The term “cultured and grow on an electrospun fiber construct” includes traditional cell culture methods as well as placing on a surface in a suitable setting, such as in natural or synthetic biocompatible matrices or tissue.

Examples of biological cells suitable for culturing and growing on the electrospun fiber constructs include stem cells, adipose derived stem cells, dental pulp stem cells, fibroblasts, and dorsal root ganglia, such as progenitor cells and adult stem cells derived or isolated from placenta, bone marrow, adipose tissue, blood vessel, amniotic fluid, synovial fluid, synovial membrane, pericardium, periosteum, dura, peripheral blood, umbilical blood, menstrual blood, baby teeth, nucleus pulposus, brain, skin, hair follicle, intestinal crypt, neural tissue, and muscle.

### 5. Active Agents

Optionally, the electrospun fiber construct also contains one or more active agents. Typically, the one or more active agents is a bioactive factor or an extracellular matrix component that can support cell growth, or an anti-inflammatory or anti-infectious agent, or a combination thereof. Any anti-inflammatory and anti-infectious agents described above can be included in the electrospun fiber construct for culturing and growing cells.

Examples of bioactive factors suitable for use for culturing cells on the electrospun fiber constructs include, but are not limited to, an osteogenic growth factor, collagen, glycosaminoglycans, osteonectin, bone sialo protein, an osteoinductive factor, a chondrogenic factor, a cytokine, a mitogenic factor, a chemotactic factor, a transforming growth factor (TGF), a fibroblast growth factor (FGF), an angiogenic factor, an insulin-like growth factor (IGF), a platelet-derived growth factor (PDGF), an epidermal growth factor (EGF), a vascular endothelial growth factor (VEGF), a nerve growth factor (NGF), a neurotrophin, a bone morphogenetic protein (BMP), osteogenin, osteopontin, osteocalcin, cementum attachment protein, erythropoietin, thrombopoietin, tumor necrosis factor (TNF), an interferon, a colony stimulating factor (CSF), stem cell derived factor-1 (SDF-1), and an interleukin. The bioactive factor may be a BMP, PDGF, FGF, VEGF, TGF, and insulin. Examples of BMPs include, but are not limited to, BMP-2, BMP-3, BMP-4, BMP-5, BMP-6, BMP-7, BMP-8, BMP-9, BMP-10, BMP-11, BMP-12, BMP-13, BMP-14, BMP-15, truncated BMPs described in PCT/US2012/053584, which is incorporated by reference in its entirety herein, and a combination thereof.

Examples of extracellular matrix components suitable for use for culturing cells on the electrospun fiber constructs include, but are not limited to, collagen, glycosaminoglycans, osteocalcin, osteonectin, bone sialo protein, osteopontin, fibronectin, laminin, vitronectin, elastin, and a combination thereof.

### 6. Cell Adhesion and Retention

Typically, the electrospun fiber construct(s) for forming an engineered tissue or implant has a grammage and open area suitable for cells to adhere to and retain on the polymer fiber bundles of the fiber construct.

Cell adhesion and retention on the fiber construct can be evaluated by counting the number of cells per microscope field. For example, the electrospun fiber constructs have a grammage and open area sufficient for cells to adhere and retain such that a desired number of cells is achieved on the fiber construct at about 2 hours, about 5 hours, about 12 hours, about 24 hours, about 36 hours, at about 48 hours, about 60 hours, or about 72 hours following cell culture.

For example, at about 2 hours, about 5 hours, about 12 hours, about 24 hours, about 36 hours, at about 48 hours, about 60 hours, or about 72 hours following cell culture, the average number of cell per microscope field (magnification 10×, field of view 1600μ×1300 μm) on the fiber construct is at least 150 per field, at least 160 per field, at least 165 per field, at least 170 per field, at least 175 per field, at least 180 per field, at least 185 per field, at least 190 per field, at least 200 per field, at least 220 per field, or at least 230 per field. The term “average number of cell per microscope field” can be determined using a standard technique, such as using ImageJ™ using the cell counter plug-in, using colormetric/fluorimetric agent and quantifying cells according to a standard curve of cell count, colorimetric quantification of enzyme released by cells in relation to cell number. Optionally, the “average number of cell per microscope field” are determined using ImageJ™ using the cell counter plug-in.

Without being bound to theories, cell adhesion and retention on the fiber construct increases with an increase of the open area of the fiber construct. From a macroscopic perspective, cells favor a more porous design polymer construct for in-migration, attachment, survival and growth. Additionally, cell adhesion and retention on the fiber construct increases with an increase of the grammage of the fiber construct. As cells functionally migrate within the fiber construct having adequate substratum adhesion surfaces and points of attachment is needed. Creating a denser, yet open, fiber construct provides a more effective distribution of underlying surface area for points of cell attachment.

Optionally, the electrospun fiber construct(s) for forming an engineered tissue or implant has a contact angle suitable for cells to adhere to and retain on the polymer fiber bundles of the fiber construct. Without being bound to theories, increasing the overall hydrophobicity of the fiber construct surface creates a less favorable environment for cell adhesion and retention. For example, the electrospun fiber construct has a contact angle of less than 120°, less than 110°, in a range from about 0° to about 120°, from about 0° to about 110°, from about 10° to about 120°, from about 20° to about 120°, from about 30° to about 120°, from about 40° to about 120°, from about 50° to about 120°, from about 60° to about 120°, from about 70° to about 120°, from about 80° to about 120°, from about 90° to about 120°, or from about 90° to about 110°, such as about 100°.

### 7. Degradation Rate

The fiber construct forming the engineered tissue or organ can degrade in vivo once the tissues or organs have been replaced or repaired by natural structures and cells.

The degradation rate of the electrospun fiber construct in vivo may be characterized by the amount of fiber construct mass lost per unit of time, such as 1 hour, 1 day, 1 week, 1 month, 3 months, 6 months, or 1 year. For example, the electrospun fiber construct loses about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of its initial mass in 1 hour, 1 day, 1 week, 1 month, 3 months, 6 months, or 1 year following its placement on a subject, such as a mammal.

Optionally, the degradation rate of the electrospun fiber construct in vivo may be characterized by the loss of strength of the fiber construct per unit of time, such as the loss of ultimate tensile strength (UTS) of the fiber construct per unit of time. For example, the electrospun fiber construct loses about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of its initial UTS in 1 hour, 1 day, 1 week, 1 month, 3 months, 6 months, or 1 year following its placement on a subject, such as a mammal.

### B. Wound Dressings

Optionally, the electrospun fiber construct is used for forming a wound dressing. In these embodiments, the electrospun fiber construct contains polymeric fibers formed by one or more biocompatible polymers and optionally one or more active agent(s).

### 1. Form of Fiber Construct

The electrospun fiber construct forming a wound dressing can have any suitable 2-D or 3-D shape and any suitable area, as long as it can cover a wound or a portion of the wound. The wound may be located on the surface of the body of a subject, such as a mammal, or in a body cavity that is open to the outside environment, such as an oral cavity and a nasal cavity.

For example, the electrospun fiber construct is in the form of a patch of any suitable shape having an area sufficient to cover the wound or a portion of the wound. The term “patch” refers to a piece or segment of biomaterial that can be placed on and/or affixed to a target anatomical structure, such as a soft tissue, to treat, protect, repair and/or reinforce a target site. The patch can be any geometric shape but is typically substantially planar and may, in position, conform to the shape of an underlying or overlying tissue.

The electrospun fiber construct patch can have a thickness in a range from about 10 μm to about 0.1 mm, from about 20 μm to about 0.1 mm, from about 30 μm to about 0.1 mm, from about 50 μm to about 0.1 mm, from about 10 μm to about 50 μm, from about 15 μm to about 50 μm, from about 20 μm to about 50 μm, from about 10 μm to about 40 μm, from about 20 μm to about 40 μm, or from about 20 μm to about 30 μm.

### 2. Exemplary Polymers

Polymers suitable for forming polymeric fibers of the electrospun fiber constructs that form wound dressings can be any biocompatible polymers described above. Optionally, the electrospun fiber constructs forming the wound dressings are formed from biodegradable polymers (e.g. natural polymers and biodegradable synthetic polymers) and/or non-biodegradable polymers (e.g. non-biodegradable synthetic polymers). These are known in the art and described above.

For example, electrospun fiber constructs for use as wound dressings can be formed from one or more of the following polymers: dextran, glycosaminoglycans, polyalginate, polylysine, hyaluronic acid, polyglycolic acid, polylactic acid, polyacrylic acid, poly-ε-caprolactone, polyvinylpyrrolidone, polyvinyl alcohol, polyethylene glycol, polyurethane, gelatin, keratin, fibrin, chitosan, pectin, collagen, alginates, agar, or cellulose, or a combination thereof.

### 3. Active Agents

Optionally, the electrospun fiber construct also contains one or more active agents. The one or more active agents can be one or more cell(s), one or more cell growth factors, one or more anesthetic agents, one or more anti-scarring, anti-inflammatory, or anti-infectious agents, or a combination thereof. Any growth factors, anesthetic agents, anti-scarring agents, anti-inflammatory agents, and anti-infectious agents described above can be included in the electrospun fiber construct for forming the wound dressing.

The active agent(s) can be coated on the surface of the electrospun fiber construct and/or embedded in the fiber construct. Optionally, the active agent(s) is encapsulated in particles and the particles are coated on the surface of the electrospun fiber construct and/or embedded in the Examples of cells suitable for use on/in the electrospun fiber constructs forming a wound dressing include, but are not limited to, cells, such as progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, or precursor cells, or a combination thereof.

### 4. Properties

The wound dressing formed by one or more electrospun fiber construct(s) can be characterized by water absorption content, equilibrium water content, oxygen transmission rate, and/or water vapor transmission rate.

For example, the electrospun fiber construct forming the wound dressing has a water absorption content of at least 100%, at least 150%, at least 200%, at least 250%, at least 300%, at least 350%, at least 400%, or at least 450%, an equilibrium water content of at least 20%, at least 30%, at least 40%, at least 50%, at least 60%, at least 70%, or at least 80%, an oxygen transmission rate of at least 5000 cm³·m⁻²·24 h⁻¹, at least 6000 cm³·m⁻²·24 h⁻¹, at least 7000 cm³·m⁻²·24 h⁻¹, at least 8000 cm³·m⁻²·24 h⁻¹, at least 9000 cm³·m⁻²·24 h⁻¹, at least 10,000 cm³·m⁻²·24 h⁻¹, or at least 15,000 cm³·m⁻²·24 h⁻¹, and/or a water vapor transmission rate of at least 1500 g·m⁻²·24 h⁻¹, at least 1800 g·m⁻²·24 h⁻¹, at least 2000 g·m⁻²·24 h⁻¹, at least 2200 g·m⁻²·24 h⁻¹, or at least 2500 g·m⁻²·24 h⁻¹. Methods for measuring the water absorption content, equilibrium water content, oxygen transmission rate, and water vapor transmission rate of an electrospun fiber construct are known, see e.g. U.S. Pat. No. 8,728,498 to Zhang, et al.; Lamke, et al., Burns, 3:159 (1977); Yan, et al., Fibers and Polymers, 12:207-213 (2011); Gaylor, et al., Biomaterials, 8:5 (1987); Letha, et al., The Journal of The Textile Institute, (2021); Wang, et al., ACS Sustainable Chemistry & Engineering, 6 (2018); and Wharram, et al., Macromolecular Bioscience, (2010).

### C. Electrospun Fiber Constructs for Drug Delivery

In some embodiments, the electrospun fiber construct contains one or more active agents suitable for delivery, typically in a controlled manner, to a patient in need thereof. Optionally, the electrospun fiber constructs are included in a drug formulation. In these embodiments, the electrospun fiber construct contains polymeric fibers formed by one or more biocompatible polymer(s) and one or more active agent(s), and optionally one or more excipient(s). The electrospun fiber construct can be in any suitable form as long as the formed formulation can be delivered to a subject, such as a mammal, via a suitable administration route.

### 1. Exemplary Polymers

Polymers suitable for forming polymeric fibers of the electrospun fiber constructs that form drug delivery formulations can be any biocompatible polymers described above. Optionally, the electrospun fiber constructs forming the drug delivery formulations are formed from biodegradable polymers (e.g. natural polymers and biodegradable synthetic polymers) and/or non-biodegradable polymers (e.g. non-biodegradable synthetic polymers).

Optionally, the polymeric fibers of the electrospun fiber construct are formed by two or more different polymers. Each of the two or more polymers can be a degradable polymer or a non-degradable polymer. Examples of polymers suitable for forming electrospun fiber constructs for use in drug delivery formulations include, polyhydroxyalkanoate, polyglycerol sebacate, polyvinyledene fluoride (“PVDF”), PVDF-co-HFP, terpolymer of PTFE-co-PVDF-co-HFP, polyethyleneterephthalate (PET), polyvinylchloride (PVC), polyesters such as poly(ethylene terephthalate), polyamides such as nylon, polyacrylonitriles, polyphosphazines, polylactones such as polycaprolactone, polyanhydrides such as polybis(p-carboxphenoxy)propane anhydride, polyalkylsulfones, polycarbonate polymers and copolymers, polyhydroxybutyrates, polyhydroxyvalerates and their copolymers, polyurethanes, hydrocarbon copolymers, polyethylene, polypropylene, polyvinyl chloride and ethylene vinyl acetate, polyorthoesters, polyanhydrides, polytyrosine, PHBV, polylactide, polylactic acid, polyglycolide, copolymers of lactide and glycolide (“PLGA”), polyhydroxybutyrate, copolymers of lactic acid and lactone, copolymers of lactic acid and PEG (“PLLA-co-PEG”), copolymers of a-hydroxy acids and C-amino acids (polydepsipeptides), polyanhydrides, polyorthoesters, polyphosphazenes, copolymers of hydroxybutyrate and hydroxyvalerate, homopolymers and copolymers of delta-valerolactone, p-dioxanone, and their copolymers with caprolactone, poly (ethylene carbonate), copoly(ethylene carbonate), polyethylene terephthalate, polycaprolactone (“PCL”), poly(acrylic acids) (i.e., Carbopols™), poly(acrylates), polyacrylamides, polyvinyl alcohols, polyethylene glycols, polyethylene vinyl acetates, poly(oxyalkene) polymers and copolymers such as poly(ethylene oxide)-poly(propylene oxide) (PEO-PPO) copolymers, and copolymers and blends of these polymers with polymers such as poly(alpha-hydroxy acids), including but not limited to lactic, glycolic and hydroxybutyric acids, polycaprolactones, and polyvalerolactones, and a copolymer thereof.

For example, the polymers for forming electrospun fiber constructs for use in drug delivery formulations are lactide homopolymers, such as poly(L-lactide), poly(D,L-lactide), copolymers of lactide and glycolide, poly(oxyethylene oxide)(“PEO”), poly(oxyethylene)-poly(oxypropylene), PEO-PPO block copolymers such as triblock PEO-PPO-PEO copolymers (POLOXAMERS™, PLURONICS™), poly (ethylene oxide)-poly(butylene oxide) (PEO-PBO), tetra-functional block copolymers derived from the sequential addition of propylene oxide and ethylene oxide to ethylene diamine (POLOXAMINES™, TETRONICS™), copolymers of PEG with poly(lactic acid), oligomers of poly(lactic acid), lactides, copolymers of PEG and amino acids, or conjugates of PEG with polysaccharides.

Optionally, the polymeric fibers of the electrospun fiber construct is formed by a single polymer, the polymeric fibers having the same as or different diameters. Optionally, the polymeric fibers of the electrospun fiber construct is formed by two or more different polymers, the polymeric fibers having the same as or different diameters.

The incorporation of different polymers and/or polymeric fibers having different diameters can achieve multiphased drug delivery due to the different degradation rates of the polymeric fibers.

a. Polymer Types

Optionally, the polymeric fibers of the electrospun fiber construct are formed by two or more different polymers, each polymer having an in vivo degradation rate as described above. Optionally, the two or more different polymers forming the polymeric fibers of the electrospun fiber construct have the same or similar degradation rate or different degradation rates. For example, the polymeric fibers of the electrospun fiber construct are formed by two or more different polymers, at least one polymer having a degradation rate that is different than the other polymers, i.e. degrades faster or slower than the other polymers in vivo. The combination of two different polymers for forming polymeric fibers can achieve multiphased drug delivery due to the different degradation rates of the polymers.

For example, the polymeric fibers are formed from two different polymers, where a first set of polymeric fibers is formed from a first polymer and a second set of polymeric fibers is formed from a second polymer. The first polymer has a first degradation rate and the second polymer has a second degradation rate. The first degradation rate is higher or lower than the second degradation rate in vivo. For example, the first polymer forming a first set of polymeric fibers can be a natural polymer, such as gelatin, keratin, fibrin, chitosan, pectin, collagen, alginates, agar, or cellulose, and the second polymer forming a second set of polymeric fibers can be PLGA, polyglycolic acid, polylactic acid, polyacrylic acid, poly-ε-caprolactone, polyvinylpyrrolidone, polyvinyl alcohol, polyethylene glycol, or polyurethane, or a copolymer thereof.

Optionally, the first set of polymeric fibers has a first degradation rate that is at least 2 times higher, at least 4 times higher, at least 5 times higher, at least 10 times higher, at least 15 times higher, at least 20 times higher, at least 25 times higher, at least 30 times higher, at least 35 times higher, at least 40 times higher, at least 45 times higher, at least 50 times higher, at least 100 times higher, at least 150 times higher, at least 200 times higher than s second degradation rate of the second set of polymeric fibers.

For example, the first set of polymeric fibers loses about 90% of its initial mass in about 1 week, about 2 weeks, about 3 weeks, about 1 month, about 2 months, or about 3 months, and the second set of polymeric fibers loses about 90% of its initial mass in about 6 month, about 7 months, about 8 months, about 10 months, about 12 months, about 15 months, about 18 months, or about 24 months.

b. Polymer Fiber Diameter

Polymeric fibers formed from the same polymer can have the same, similar, or different diameters. For example, the polymeric fibers of the fiber construct are formed from a single polymer, the polymeric fibers have the same or a similar diameter in a range as described above.

Optionally, polymeric fibers formed from the same polymer have different diameters. For example, the polymeric fibers of the electrospun fiber construct are formed by the same polymer, where a first set of polymeric fibers has a first diameter that is greater or smaller than a second diameter of a second set of polymeric fibers. For example, the first diameter of the first set of polymeric fibers is in a range from about 10 nm to about 1 μm, such as from about 20 nm to about 1 μm, from about 50 nm to about 1 μm, from about 100 nm to about 1 μm, from about 10 nm to about 0.5 μm, from about 20 nm to about 0.5 μm, from about 50 nm to about 0.5 μm, from about 0.1 μm to about 1 μm, or from about 0.5 μm to about 1 μm, and the second diameter of the second set of polymeric fibers has a second diameter in a range from about 1 μm to about 10 μm, such as from about 1 μm to about 9 μm, from about 1 μm to about 8 μm, from about 1 μm to about 7 μm, from about 1 μm to about 6 μm, from about 1 μm to about 5 μm, or from about 2 μm to about 8 μm.

Optionally, the polymeric fibers of the fiber construct are formed from two or more different polymers, where each polymer forms a set of polymeric fibers having a different diameter. For example, the polymeric fibers of the fiber construct are formed from two different polymers, where a first set of polymeric fibers is formed from a first polymer and a second set of polymeric fibers is formed from a second polymer. The first set of polymeric fibers has a first diameter that is different from a second diameter of the second set of polymeric fibers.

### 2. Active Agents

The electrospun fiber construct forming a drug delivery formulation also contains one or more active agents. The one or more active agents can be any of the active agents described above. Examples of active agents suitable for use in drug delivery formulations formed by electrospun fiber construct include anti-inflammatory drugs, antibiotics, chemotherapeutics, antimitotics, anti-proliferative drugs, antibodies, prothrombotics, antithrombotics, and growth factors.

The active agent(s) can be coated on the surface of the electrospun fiber construct and/or embedded in the fiber construct. Optionally, the active agent(s) is associated with a carrier, such as particles, e.g. polymeric particles, metallic particles, lipid particles, and dendrimers. In these embodiments, the active agent(s) can be complexed with, covalently attached to, or encapsulated in the particles. The resulting particles can be coated on the surface of the electrospun fiber construct and/or embedded in the fiber construct.

Optionally, the particles for use in these embodiments has a size sufficient to allow encapsulation of an active agent described above and/or to allow embedment in the fiber construct. For example, the particles have an average diameters of less than 10 μm, less than 5 μm, less than 1 μm, less than 500 nm, less than 200 nm, less than 100 nm, up to 20 μm, up to 15μ, in a range from about 20 nm to about 20 μm, from about 50 nm to about 20 μm, from about 20 nm to about 10 μm, from about 50 nm to about 10 μm, from about 20 nm to about 5 μm, from about 50 nm to about 5 μm, from about 20 nm to about 2 μm, from about 50 nm to about 2 μm, from about 20 nm to about 1 μm, from about 50 nm to about 1 μm, from about 20 nm to about 0.5 μm, from about 50 nm to about 0.5 μm, from about 20 nm to about 0.1 μm, or from about 50 nm to about 0.1 μm.

a. Embedment Location

When a single polymer is used for forming polymeric fibers of the fiber construct, the same as or different active agent(s) and/or active agent(s) associated with particles can be embedded into polymeric fibers having the same or similar diameter or different fiber diameters. For example, the same active agent and/or active agent associated with particles are embedded into polymeric fibers having the same or similar diameter. For example, each active agent of two or more different active agents or active agents associated with particles is embedded into a set of polymeric fibers having a fiber diameter that is different from the other sets of polymeric fibers of the fiber construct.

When different polymers are used for forming polymeric fibers of the fiber construct and/or polymeric fibers formed have different diameters, the active agent(s) can be released at different rates achieve multiphased drug delivery due to the different degradation rates of the polymeric fibers.

For example, the active agent(s) and/or active agent(s) associated with particles can be embedded into polymeric fibers formed from different polymers and/or having different fiber diameters. The same active agent and/or active agent associated with particles can be embedded into polymeric fibers formed from different polymers and/or having different fiber diameters. Alternatively, active agent and/or active agent associated with particles can be embedded into polymeric fibers formed from different polymers and/or having different fiber diameters.

Typically, polymeric fibers having a larger diameter degrade faster in vivo and hence provide sustained release compared to polymeric fibers having a smaller diameter. Accordingly, active agent(s) embedded in the polymeric fibers having a larger diameter relative to the other polymeric fibers of the fiber construct is released faster from the fiber construct.

For example, the electrospun fiber construct contains polymeric fibers having two different diameters and one active agent, the active agent may be in a free form or associated with a carrier. A first set of polymeric fibers of the fiber construct has a first diameter and a second set of polymeric fibers of the fiber construct has a second diameter, where each set of polymeric fibers may be formed from the same polymer or a different polymer. As such, the first set of polymeric fibers have a first degradation rate that is different from the second set of polymeric fibers. The active agent is embedded in the first set of polymeric fibers having the first diameter and in the second set of polymeric fibers having the second diameter. Accordingly, the active agent embedded in the first set of polymeric fibers is released from the fiber construct at a release rate that is different (i.e. lower or greater) from the active agent embedded in the second set of polymeric fibers in vivo.

For example, the electrospun fiber construct contains polymeric fibers having two different diameters and two different active agents, where each active agent may be in a free form or associated with a carrier. A first set of polymeric fibers of the fiber construct has a first diameter and a second set of polymeric fibers of the fiber construct has a second diameter, where each set of polymeric fibers may be formed from the same polymer or a different polymer. As such, the first set of polymeric fibers have a first degradation rate that is different from the second set of polymeric fibers. A first active agent is embedded in the first set of polymeric fibers having the first diameter and a second active agent is embedded in the second set of polymeric fibers having the second diameter. Accordingly, the first active agent is released from the fiber construct at a release rate that is different (i.e. lower or greater) from the second active agent in vivo.

For example, the electrospun fiber construct contains polymeric fibers formed from two different polymers and one active agent, the active agent may be in a free form or associated with a carrier. A first set of polymeric fibers formed from a first polymer and a second set of polymeric fibers formed from a second polymer may have the same as or different diameters. As such, the first set of polymeric fibers has a first degradation rate that is different from the second set of polymeric fibers. The active agent is embedded in the first set of polymeric fibers formed from the first polymer and in the second set of polymeric fibers formed from the second polymer. Accordingly, the active agent embedded in the first set of polymeric fibers is released from the fiber construct at a release rate that is different (i.e. lower or greater) from the active agent embedded in the second set of polymeric fibers in vivo.

For example, the electrospun fiber construct contains polymeric fibers formed from two different polymers and two different active agents, where each active agent may be in a free form or associated with a carrier. A first set of polymeric fibers formed from a first polymer and a second set of polymeric fibers formed from a second polymer may have the same as or different diameters. As such, the first set of polymeric fibers has a first degradation rate that is different from the second set of polymeric fibers. A first active agent is embedded in the first set of polymeric fibers formed from the first polymer and a second active agent is embedded in the second set of polymeric fibers formed from the second polymer. Accordingly, the first active agent is released from the fiber construct at a release rate that is different (i.e. lower or greater) from the second active agent in vivo.

### 3. Excipients

The drug delivery formulation may contain the electrospun fiber construct and one or more excipient(s) to form an oral formulation, a parenteral formulation, a pulmonary or mucosal formulation, or a topical formulation.

Optionally, the fiber construct may include one or more excipients, such as drug solubility modifiers, excipients that co-release with the active agent(s) to enhance drug bioavailability to the target tissue, and/or drug stability enhancers, or a combination thereof.

Optionally, the drug delivery formulation contains the electrospun fiber construction and one or more drug solubility modifiers, excipients that co-release with the active agent(s) and enhance drug bioavailability to the target tissue, and/or drug stability enhancers. The formulation may be an oral formulation, parenteral formulation, pulmonary or mucosal formulation, or topical formulation.

a. Excipients for Oral Formulation

The drug delivery formulation formed from the electrospun fiber construct can be in a form suitable for oral administration. Oral administration may involve swallowing, so that the active agent(s) coated on or embedded in the electrospun fiber construct enters the gastrointestinal tract, or buccal or sublingual administration may be employed by which the active agent(s) coated on or embedded in the electrospun fiber construct enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

Optionally the electrospun fiber construct is included in a fast-dissolving, fast-disintegrating dosage form.

For tablet or capsule dosage forms, depending on dose, the active agent(s) coated on or embedded in the electrospun fiber construct may make up from 1 weight % to 80 weight % of the dosage form, more typically from 5 weight % to 60 weight % of the dosage form. In addition to the electrospun fiber construct described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), anti-oxidants, colourants, flavouring agents, preservatives and taste-masking agents.

Exemplary tablets contain up to about 80% of the electrospun fiber construct described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations for oral administration may be formulated to be immediate and/or modified release. Modified release formulations include delayed, sustained, pulsed, controlled, targeted and programmed release formulations.

b. Excipient for Parenteral Formulations

The electrospun fiber construct can also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the electrospun fiber construct used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations.

c. Excipients for Pulmonary and Mucosal Formulations

The electrospun fiber construct can be included in a formulation suitable for pulmonary or mucosal administration. The administration can include delivery of the composition to the lungs, nasal, oral (sublingual, buccal), vaginal, or rectal mucosa.

For example, the electrospun fiber construct can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may contain a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the electrospun fiber construct is micronised to a size suitable for delivery by inhalation (typically less than 5 microns). This may be achieved by any appropriate comminuting method, such as laser cutting, lithography, spiral jet milling, fluid bed jet milling, supercritical fluid processing to form electrospun fiber construct of a desired length.

Capsules (made, for example, from gelatin or hydroxypropylmethylcellulose), blisters and cartridges for use in an inhaler or insufflator may be formulated to contain a powder mix of the electrospun fiber construct described herein, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the electrospun fiber construct per actuation and the actuation volume may vary from 1 μl to 100 μl. A typical formulation may contain one or more of the electrospun fiber construct described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff”. The overall daily dose will be administered in a single dose or, more usually, as divided doses throughout the day.

In some forms, the electrospun fiber construct can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the drug delivery formulation may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

d. Excipients for Topical Formulation

The drug delivery formulation containing the electrospun fiber construct can be a form suitable for administration to the external surface of the skin or the mucous membranes (including the surface membranes of the nose, lungs and mouth). For example, these topical formulations may deliver one or more active agent(s) that are coated on or embedded in the electrospun fiber construct cross the external surface of the skin or mucous membrane and thereby enter the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

### 4. Dosages of Active Agents

The drug delivery formulation contains an effective amount of the active agent(s) coated on or embedded in the electrospun fiber construct. The effective amount depend on many factors, including the indication being treated, the route of administration, co-administration of other therapeutic compositions, and the overall condition of the patient.

Exemplary effective amount of the active agent(s) contained in the drug delivery formulation (in unit dosage form) can be from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg.

### D. Implant/Device Coatings

In some embodiments, the electrospun fiber construct is for a coating of an implant or medical device. In these embodiments, the electrospun fiber construct contains polymeric fibers formed by one or more biocompatible polymers and optionally one or more active agent(s).

In these embodiments, the fiber construct can be in the form of a patch or a 3-D shape. Typically, the patch has a planar shape that conforms to a surface or a portion of a surface of the implant or medical device. When the fiber construct is in the form of a 3-D shape, the shape can be defined by three boundaries and have a volume inside the boundaries. The volume of the 3-D shaped fiber construct is sufficient to surround an implant or medical device.

The electrospun fiber construct coating can have a thickness in a range from about 10 μm to about 0.1 mm, from about 20 μm to about 0.1 mm, from about 30 μm to about 0.1 mm, from about 50 μm to about 0.1 mm, from about 10 μm to about 50 μm, from about 15 μm to about 50 μm, from about 20 μm to about 50 μm, from about 10 μm to about 40 μm, from about 20 μm to about 40 μm, or from about 20 μm to about 30 μm.

### 1. Exemplary Polymers

Polymers suitable for forming polymeric fibers of the electrospun fiber constructs for use as a coating can be any biocompatible polymers described above. Optionally, the electrospun fiber constructs forming the coating are formed from biodegradable polymers (e.g. natural polymers and biodegradable synthetic polymers) as described above.

Examples of polymers suitable for forming electrospun fiber constructs for use as coating include PVDF-HFP, PC polymer, and heparin-PEG blend, and a combination thereof.

### 2. Active Agents

Optionally, the electrospun fiber construct also contains one or more active agents. The one or more active agents can be one or more cell(s), one or more growth factors, one or more anesthetic agents, one or more anti-inflammatory or anti-infectious agents, or a combination thereof. Any cells, growth factors, anesthetic agents, anti-inflammatory agents, and anti-infectious agents described above can be included in the electrospun fiber construct for forming the coating on an implant or medical device.

The active agent(s) can be coated on the surface of the electrospun fiber construct and/or embedded in the fiber construct. Optionally, the active agent(s) is encapsulated in particles and the particles are coated on the surface of the electrospun fiber construct and/or embedded in the

### 3. Exemplary Medical Devices

Examples of implants and medical devices that may be coated by the electrospun fiber construct include, but are not limited to, a stent, graft, fistula, occlusion device, cavity filling device, tissue expander, barrier device, pacemaker, defibrillator, VAD, artificial heart, insulin pump, nerve stimulator, prosthetic devices (such as artificial hip joints, artificial ligaments, artificial tendons and artificial knee joints), cardiovascular devices (such as vascular grafts, artificial heart valves and stents), anatomical structures (such as anatomical bone, tooth, nerves, pancreas, eye and muscle), implantable biosensors (such as those used to monitor the level of drugs within a living body, or the level of blood glucose in a diabetic patient) and percutaneous devices (such as catheters).

### V. Methods of Using the Electrospun Fiber Constructs

Methods of using the electrospun fiber constructs are described herein. The electrospun fiber constructs can have a variety of different uses on their own or as a part of a composition, such as tissue engineering, wound healing, and drug delivery.

### A. Tissue Engineering

The electrospun fiber constructs may be used for repairing or replacing tissue or organ in a subject in need thereof. The subject may be a mammal.

Generally, the method includes (i) administering an engineered tissue or implant to a site in a subject in need thereof. The engineered tissue or implant contains one or more electrospun fiber constructs. The electrospun fiber construct(s) forming the engineered tissue or implant contains polymeric fibers formed by one or more biocompatible polymer(s) and optionally one or more active agent(s).

The engineered tissue or implant may be administer to the site in the subject via a stent, a catheter, a trocar, or a needle, typically by a medical professional.

Optionally, the method also includes attaching cells on the electrospun fiber construct and optionally culturing the cells prior to step (i). The cells may be cultured using a traditional cell culture method. For example, following attachment, the cells on the electrospun fiber construct are placed in an environment suitable for growing cells for a period in a range from about 15 minutes to about 4 weeks, from about 2 hours to about 2 weeks, from about 2 hours to about 1 week, from about 2 hours to about 96 hours, from about 2 hours to about 72 hours, from about 2 hours to about 60 hours, from about 2 hours to about 48 hours, from about 2 hours to about 36 hours, from about 12 hours to about 72 hours, from about 12 hours to about 60 hours, from about 12 hours to about 48 hours, from about 12 hours to about 36 hours, such as about 24 hours. The term “environment suitable for growing cells” refers to a temperature in a range from about 20° C. to about 40° C., such as about 37° C. and an atmosphere containing between about 1% CO₂ and about 10% CO₂.

Optionally, the method includes sterilizing the engineered tissue or implant prior to step (i). Sterilization methods are known in the art. For example, a user, such as a human, washes the engineered tissue or implant using an ethanol, or exposes the engineered tissue or implant under UV light for a period sufficient for sterilization, such as between about 1 minute to about 1 hour, between about 5 minutes to about 1 hour, between about 10 minutes to about 1 hour, or between about 10 minutes to about 30 minutes.

### B. Wound Healing

The electrospun fiber constructs may be used for promoting wound heating for a subject in need thereof. The subject may be a mammal.

Generally, the method includes (i) applying at least one wound dressing on a wound or a portion thereof. The wound dressing contains one or more electrospun fiber constructs. The electrospun fiber construct(s) forming the wound dressing contains polymeric fibers formed by one or more biocompatible polymer(s) and optionally one or more active agent(s).

The wound may be located on the surface of the body of a subject, such as a mammal, or in a body cavity that is open to the outside environment, such as an oral cavity and a nasal cavity. The wound dressing may be applied by a medical professional or the subject being treated (e.g. self-administration).

Optionally, the method includes sterilizing the wound dressing prior to step (i). For example, a user, such as a human, washes or wipes the wound dressing prior to applying it on the wound or a portion thereof.

### C. Drug Delivery

The electrospun fiber constructs may be used for delivering an active agent, such as a drug or two or more drugs, a subject in need thereof. The subject may be a mammal.

Generally, the method includes (i) administering to the subject a drug delivery formulation. The drug delivery formulation contains one or more electrospun fiber constructs. The electrospun fiber construct(s) forming the drug delivery formulation contains polymeric fibers formed by one or more biocompatible polymer(s) and one or more active agent(s), and optionally one or more excipients.

The drug delivery formulation may be administered to the subject by oral administration, parenteral administration, inhalation, mucosal administration, topical administration, or a combination thereof. The drug delivery formulation may be applied by a medical professional or the subject being treated (e.g. self-administration).

### 1. Release Rate

Following administration, the one or more active agent(s) can be released from the formulation for up to 2 years, up to 18 months, up to 1 year, up to 8 months, up to 6 months, up to 5 months, up to 4 months, up to 3 months, up to 2 months, up to 1 month, up to 2 weeks, up to 1 week, up to 72 hours, up to 60 hours, up to 48 hours, up to 36 hours, up to 24 hours, up to 12 hours, up to 10 hours, at least 5 hours, at least 3 hours, at least 2 hours, at least 1 hour, from 1 day to 2 years, from 1 day to 1 year, from 1 week to 1 year, from 1 month to 1 year, from 3 months to 1 year, from 6 month to 2 years, from 6 months to 1 year, from 1 day to 1 month, from 1 day to 2 weeks, from 1 day to 1 week, from 1 hour to 72 hours, from 1 hour to 60 hours, from 1 hour to 48 hours, from 2 hours to 48 hours, from 5 hours to 48 hours, from 1 hour to 36 hours, from 2 hours to 36 hours, from 5 hours to 36 hours, from 1 hour to 24 hours, from 2 hours to 24 hours, from 5 hours to 24 hours, from 1 hour to 12 hours, from 2 hours to 12 hours, or from 5 hours to 12 hours.

Optionally, following administration, a first active agent is released from the formulation at a first release rate and a second active agent is released from the formulation at a second release rate, and wherein the first release rate is the same or similar as or different from the second release rate. The first release rate and second release rate can be in any one of the range described above.

For example, following administration, the first active agent is released from the formulation at a release rate that is higher than the second active agent. For example, following administration, the first active agent is released from the formulation at a release rate that is lower than the second active agent.

For example, following administration, the first active agent is released from the formulation from about 1 hour to about 1 day and the second active agent is released from the formulation from about 1 hour to about 1 month or from about 1 day to about 1 month.

For example, following administration, the first active agent is released from the formulation from about 1 day to about 1 month and the second active agent is released from the formulation from about 1 day to about 1 year or from about 1 month to about 1 year.

The disclosed compositions, systems, and methods can be further understood through the following numbered paragraphs.
1. A system for generating fiber constructs via electrospinning comprising an ejection device and a collector, wherein the collector comprises a collection surface comprising
   a conductive trace or a plurality of conductive traces; and
   a non-conductive region or a plurality of non-conductive regions,
   wherein the conductive trace or the plurality of conductive traces form(s) a pattern on the collection surface,
   wherein the collector has a conductive trace area from about 5% to about 99% of the total of the conductive trace area and a non-conductive area, optionally from about 20% to about 80% of the total of the conductive trace area and non-conductive area.
2. The system of paragraph 1, wherein the pattern on the collector comprises lines, checkerboard, squares, spirals, helices, zigzags, zigzags, sinusoidal curves, non-sinusoidal wave forms, or circles, or a combination thereof.
3. The system of paragraph 1 or 2, wherein in use when an electrostatic charge is applied to the ejection device or a component thereof, the conductive trace(s) in the pattern is arranged such that the resulting electric field lines are either undisturbed electrostatic field lines or blurred electrostatic field lines, or a combination thereof, aligned around the conductive trace or each conductive trace of the plurality of conductive traces.
4. The system of any one of paragraphs 1-3, wherein the ejection device comprises a reservoir and an ejector, and optionally the system further comprises a power supply, a pressurization component, a temperature control component, a position control component, one or more sensors, a controller, or an output device, or a combination thereof.
5. The system of paragraph 4, wherein the ejector is a needle having an inner diameter in a range from about 0.08 mm to about 4.5 mm.
6. A method of generating a fiber construct using the electrospinning system of any one of paragraphs 1 to 3 or 5, wherein the ejection device comprises a reservoir and an ejector, and wherein the reservoir comprises a polymer solution contained therein;
   the method comprising
   (ia) selecting a collector, wherein the collector has a collection surface comprising a pattern sufficient to generate the fiber construct having a grammage that is at least 5% higher than a grammage of a fiber construct formed on an un-patterned collector under the same conditions; and
   (i) applying an electrostatic charge to the ejection device or a component thereof,
   wherein following step (i), the polymer solution is extruded out of the ejection device via the ejector, wherein the polymer solution is directed to the collection surface, and forms the fiber construct on the collection surface of the collector.
7. A method of generating a fiber construct using the electrospinning system of any one of paragraphs 1 to 3 or 5, wherein the ejection device comprises a reservoir and an ejector, and wherein the reservoir comprises a polymer solution contained therein;
   the method comprising
   (i) applying an electrostatic charge to the ejection device or a component thereof,
   wherein following step (i), the polymer solution is extruded out of the ejection device via the ejector, wherein the polymer solution is directed to the collection surface, and forms the fiber construct on the collection surface of the collector,
   wherein the formed fiber construct has a grammage that is at least 5% higher than a grammage of a fiber construct formed on an un-patterned collector under the same conditions.
8. The method of paragraph 6 or 7, further comprising loading the polymer solution into the reservoir of the ejection device prior to step (i), wherein the polymer solution comprises one or more biocompatible polymers and a solvent, and optionally one or more active agent and/or one or more additive.
9. The method of any one of paragraphs 6-8, wherein the system further comprises a power supply, a pressurization component, a temperature control component, a position control component, one or more sensors, a controller, or an output device, or a combination thereof.
10. The method of any one of paragraphs 6-9, wherein the conductive trace or each conductive trace of the plurality of conductive traces is arranged such that during step (i), undisturbed electric field lines or blurred electric field lines align around the conductive trace or each conductive trace of the plurality of conductive traces.
11. The method of any one of paragraphs 6-10, wherein the biocompatible polymer is a natural polymer or a synthetic polymer, or a combination thereof, and wherein the molecular weight of the natural polymer or synthetic polymer is in a range from about 1×10³ g/mol to about 500×10³ g/mol.
12. The method of any one paragraphs 6-11, wherein the total concentration of the one or more polymer(s) is in a range from about 1% (w/v) to 90% (w/v) of the polymer solution.
13. The method of any one of paragraphs 6-12, wherein the polymer solution comprises two or more polymers, and wherein each polymer is present at a concentration in a range from about 0.1% (w/v) to 89.9% (w/v) of the polymer solution.
14. The method of any one of paragraphs 6-13, wherein the polymer solution is extruded from the ejection system at an extrusion rate in a range from about 0.01 mL/h to about 50 mL/h.
15. The method of any one of paragraphs 6-14, further comprising repeating steps (ia) and (i) or repeating step (i) at least one time to generate a fiber construct having at least two layers.
16. The method of any one of paragraphs 6-15, further comprising mixing the polymer with the solvent to form the polymer solution and/or assembling the system prior to step (i).
17. The method of any one of paragraphs 6-16, further comprising (ii) post-processing the fiber construct subsequent to step (i),
   wherein step (ii) comprises triggering cross-linking in the fiber construct, shaping the fiber construct, cutting the fiber construct, heating the fiber construct, depositing a coating material on the surface of the fiber construct, and/or adding one or more active agent(s) and/or one or more additive(s) onto/into the fiber construct.
18. The method of any one of paragraphs 6-17, further comprising
   loading an active agent solution or suspension in a second reservoir prior to step (ia) or subsequent to step (ia) and prior to step (i).
19. The method of any one of paragraphs 8-18, wherein at least one of the one or more active agent(s) is associated with a carrier.
20. An electrospun fiber construct comprising polymeric fibers and open spaces having a microporosity and a macroporosity, wherein the electrospun fiber construct has a smaller microporosity and/or a greater macroporosity than a control electrospun fiber construct formed under the same conditions and using a patterned collector having the same total area.
21. The electrospun fiber construct of paragraph 20, wherein when seeded with a type of cells and exposed to cell growth conditions, the adhesion of the cells to the electrospun fiber construct is greater than the adhesion of the same type of cells under the same cell growth conditions to the control electrospun fiber construct.
22. An electrospun fiber construct comprising polymeric fibers, wherein the electrospun fiber construct has a grammage in a range from about 18 μg/mm² to about 100 μg/mm².
23. The electrospun fiber construct of any one of paragraphs 20-22, having an open area from about 8% to about 99% of the total of the open area and a fiber area.
24. The electrospun fiber construct of any one of paragraphs 20-23, wherein the electrospun fiber construct comprises a biocompatible polymer, optionally more than one biocompatible polymer, and
   wherein the biocompatible polymer or optionally each biocompatible polymer is a natural polymer, a synthetic biodegradable polymer, or a synthetic non-biodegradable polymer, or a combination thereof.
25. The electrospun fiber construct of any one of paragraphs 20-24, wherein the electrospun fiber construct comprises two or more biocompatible polymers, and wherein each polymer is present in a range from about 0.1 wt % to about 90 wt % of the electrospun fiber construct.
26. The electrospun fiber construct of any one of paragraphs 20-25, wherein the polymeric fibers have the same or similar diameter, wherein the diameter of the fibers is in a range from about 10 nm to about 10 μm.
27. The electrospun fiber construct of any one of paragraphs 20-26, wherein the polymeric fibers have two or more different diameters, wherein each diameter is in a range from about 10 nm to about 10 μm.
28. The electrospun fiber construct of any one of paragraphs 20-27, wherein the electrospun fiber construct loses about 90% of its initial mass in about 1 hour, 1 day, 1 week, 1 month, 3 months, 6 months, or 1 year.
29. An engineered tissue or implant comprising an electrospun fiber construct of any one of paragraphs 20-28, wherein the electrospun fiber construct has a shape suitable to fit a defect or site of an organ or tissue of a mammal.
30. The engineered tissue or implant of paragraph 29, wherein the engineered tissue or implant has a 2-D shape selected from the group consisting of circle, oval, rectangular, square, triangle, polygonal, and an irregular shape, or a 3-D shape selected from the group consisting of a cylinder, a sphere, a cone, a cube, a polyhedron, a cuboid, an ellipsoid, a donut, and an irregular shape.
31. The engineered tissue or implant of paragraph 29 or 30 further comprising cells entrapped in the fiber area and/or open area of the electrospun fiber construct.
32. The engineered tissue or implant of paragraph 31, wherein the cells are selected from the group consisting of stem cells, adipose derived stem cells, dental pulp stem cells, fibroblasts, and dorsal root ganglia, such as progenitor cells and adult stem cells derived or isolated from placenta, bone marrow, adipose tissue, blood vessel, amniotic fluid, synovial fluid, synovial membrane, pericardium, periosteum, dura, peripheral blood, umbilical blood, menstrual blood, baby teeth, nucleus pulposus, brain, skin, hair follicle, intestinal crypt, neural tissue, and muscle, and a combination thereof.
33. The engineered tissue or implant of any one of paragraphs 29-32, wherein the electrospun fiber construct further comprises one or more active agent(s), wherein at least one active agent is a bioactive factor, optionally a growth factor such as an epidermal growth factor, an extracellular matrix component, an anti-inflammatory agent, or an anti-infectious agent, or a combination thereof, and wherein the active agent is embedded in the electrospun fiber construct or coated on the surface of the electrospun fiber construct.
34. The engineered tissue or implant of any one of paragraphs 29-33 comprising two or more electrospun fiber constructs, wherein the electrospun fiber constructs are sutured, sealed, stapled, stacked on top of, or otherwise adhered to one another, or a combination thereof, to form the shape.
35. A method of tissue repairing or organ implanting comprising
   (i) administering the engineered tissue or implant of any one of paragraphs 29-34 to a site in a subject in need thereof.
36. The method of paragraph 35 further comprising attaching cells on the electrospun fiber construct and optionally culturing the cells prior to step (i).
37. The method of paragraph 35 or 36, wherein the engineered tissue or implant is administered to the site in the subject via a stent, a catheter, a trocar, or a needle.
38. A wound dressing comprising an electrospun fiber construct of any one of paragraphs 20-28, wherein the wound dressing has a shape effective to cover a wound or a portion of a wound.
39. The wound dressing of paragraph 38, wherein the electrospun fiber construct comprises one or more active agent(s) selected from the group consisting of cells, cell growth factors, epidermal growth factors, anesthetic agents, anti-scarring agents, anti-inflammatory agent, and anti-infectious agents, and a combination thereof.
40. The wound dressing of paragraph 38 or 39, wherein the electrospun fiber construct is in the form of a patch, and wherein the patch has a thickness in a range from about 20 μm to about 100 μm.
41. A method of promoting wound healing comprising
   (i) applying at least one wound dressing of paragraphs 38-40 on a wound or a portion of a wound of a subject.
42. The method of paragraph 41 further comprising treating the wound dressing with an alcohol prior to step (i).
43. A formulation for drug delivery comprising
   an electrospun fiber construct of any one of paragraphs 20-28; and
   one or more active agent(s).
44. The formulation of paragraph 43, wherein the drug is selected from the group consisting of epidermal growth factors, anti-fibrotic/anti-scarring agents, anti-inflammatory agents, anti-infectious agents, growth factors, anti-cancer agents, anesthetic agents, prothrombotic/antithrombotic agents, and anti-allergenic agents, and a combination thereof.
45. The formulation of paragraph 43 or 44, wherein the polymeric fibers are formed from a single polymer, and wherein a first set of polymeric fibers have a first diameter in a range from about 10 nm to about 1 μm and a second set of polymeric fibers have a second diameter in a range from about 1 μm to 10 μm.
46. The formulation of paragraphs 45, wherein the first set of polymeric fibers comprises the same active agent as the second set of polymeric fibers or an active agent that is different from the second set of polymeric fibers.
47. The formulation of any one of paragraphs 43 or 44, wherein the polymeric fibers are formed from two different polymers,
   wherein a first set of polymeric fibers is formed from a first polymer selected from the group consisting of gelatin, keratin, fibrin, chitosan, pectin, collagen, alginates, agar, and cellulose, and
   wherein a second set of polymeric fibers is formed from a second polymer selected from the group consisting of polyglycolic acid, polylactic acid, polyacrylic acid, poly-ε-caprolactone, polyvinylpyrrolidone, polyvinyl alcohol, polyethylene glycol, and polyurethane.
48. The formulation of paragraph 47, wherein the first set of polymeric fibers has a diameter that is the same or similar to or different from the second set of polymeric fibers.
49. The formulation of paragraph 47 or 48, wherein the first set of polymeric fibers comprises the same active agent as the second set of polymeric fibers or an active agent that is different from the second set of polymeric fibers.
50. The formulation of any one of paragraphs 43-49, wherein the electrospun fiber construct further comprises particles embedded in the electrospun fiber construct or coated on the surface of the electrospun fiber construct, and wherein the active agent is encapsulated in or attached to the particles.
51. The formulation of any one of paragraphs 43-50 further comprising a pharmaceutically acceptable excipient.
52. A method of delivering drug(s) to a subject in need thereof comprising
   (i) administering to the subject the formulation of any one of paragraphs 43-51.
53. The method of paragraph 52, wherein step (i) is via oral administration, parenteral administration, inhalation, mucosal administration, topical application, or a combination thereof.
54. The method of 52 or 53, wherein following step (i), the drug is released from the formulation for up to 2 years, up to 18 months, up to 1 year, up to 8 months, up to 6 months, up to 5 months, up to 4 months, up to 3 months, up to 2 months, up to 1 month, up to 2 weeks, up to 1 week, up to 72 hours, up to 60 hours, up to 48 hours, up to 36 hours, up to 24 hours, up to 12 hours, up to 10 hours, at least 5 hours, at least 3 hours, at least 2 hours, at least 1 hour, from 1 day to 2 years, from 1 day to 1 year, from 1 week to 1 year, from 1 month to 1 year, from 3 months to 1 year, from 6 month to 2 years, from 6 months to 1 year, from 1 day to 1 month, from 1 day to 2 weeks, from 1 day to 1 week, from 1 hour to 72 hours, from 1 hour to 60 hours, from 1 hour to 48 hours, from 2 hours to 48 hours, from 5 hours to 48 hours, from 1 hour to 36 hours, from 2 hours to 36 hours, from 5 hours to 36 hours, from 1 hour to 24 hours, from 2 hours to 24 hours, from 5 hours to 24 hours, from 1 hour to 12 hours, from 2 hours to 12 hours, or from 5 hours to 12 hours.
55. The method of any one of paragraphs 52-54, wherein following step (i), a first active agent is released from the formulation at a first release rate and a second active agent is released from the formulation at a second release rate, and wherein the first release rate is the same as, similar to, or different from the second release rate.
56. A coating for a medical device comprising an electrospun fiber construct of any one of paragraphs 20-28.
57. The coating of paragraph 56, wherein the electrospun fiber construct comprises one or more active agents selected from the group consisting of cells, cell growth factors, epidermal growth factors, anesthetic agents, anti-scarring agents, anti-inflammatory agent, and anti-infectious agents, and a combination thereof.
58. The coating of paragraph 56 or 57, wherein the medical device is a stent, graft, fistula, occlusion device, cavity filling device, tissue expander, barrier device, pacemaker, defibrillator, ventricular assist device (VAD), artificial heart, insulin pump, nerve stimulator, prosthetic device, cardiovascular device, anatomical structure, implantable biosensor, or percutaneous device.

The present invention will be further understood by reference to the following non-limiting examples.

### EXAMPLES

### Materials and Methods

### Patterned Collector Fabrication

Five differing patterns were created for collectors: lines, squares, zigzags, sinusoids, and solid. The patterned collectors (3×1 cm each) were designed using Copper Connection PCB Editor (ExpressPCB, LLC). Designs were laser printed onto Press-N-Peel Blue Transfer Sheets (Techniks Inc, Indianapolis, IN, USA) and heat transferred to a clean, copper-clad circuit board laminate. The copper boards were submerged in an etching solution (2:1 mixture of 0.9 M H₂O₂:12 M HCl) for 40 minutes. Etched boards were then washed with acetone to remove transfer sheet material, revealing copper designs as copper traces. Individual patterns are shown in FIG. 1.

### Polymer Scaffold Fabrication

PLGA (95:5) solution was prepared in dichloromethane (DCM) as solvent at 7.5% concentration (w/v) and vortexed for 45 minutes at room temperature. The resultant homogenous solution was loaded within a horizontal syringe pump (New Era Pump Systems, Inc. Farmingdale, NY, USA). The extruding gauge needle (23 G) was connected to the cathode of a direct current power supply (12 kV) while the copper collector was grounded, creating electrical potential. With current flowing, the PLGA solution was extruded at a rate of 2.5 mL/hr jetted towards copper collector targets positioned at a distance of 15 cm (FIG. 1). A non-fibrous, i.e. continuous, PLGA sample was fabricated for reference, by spin coating PLGA solution at 500 RPM for 10 seconds using a spin coater (Model PWM32, Headway Research Inc., Garland, Texas, USA). Spin coated continuous samples were used as control for comparison to electrospun fibrous PLGA scaffolds for all studies performed.

### Scaffold Physical Characterization

The mass of fiber scaffolds was determined using an analytical balance (Mettler AE 100, Mettler Toledo, Columbus, OH, USA). Fiber diameter was determined via measurements from scanning electron microscope (SEM) images obtained using a Hitachi-S4800 field emission SEM (Chiyoda, Tokyo, Japan; 500× magnification). Scaffold images were analyzed using ImageJ™ software (NIH Bethesda, MD) to determine fiber diameter. The scale bar on each SEM image is presented as a dimensional control. High-resolution images of each collector and PLGA scaffold were taken with a Nikon D7100 DSLR camera (Nikon Inc. Melville, NY, USA) in a controlled lighting environment. Resultant images were converted to binary and analyzed in Adobe Photoshop CS5 Extended (Version 12.1, Adobe Systems Incorporated, San Jose, CA, USA) to determine pixel count of exposed copper traces, or PLGA fibers, for each design studied. A predetermined pixel:area ratio was utilized to convert pixel count to mm². To determine the surface area of the exposed copper target (copper traces) and the adherent polymer scaffold construct, as a % of the total target area (i.e. copper trace conductive area+non-conductive insulated area), the following were calculated:

$$Copper Trace Area ( \% ) = ( copper trace area \div total area ) \times 100 \qquad ( 1 )$$

$$Fiber Target Coverage ( \% ) = ( fiber ( scaffold ) area \div total area ) \times 100 \qquad ( 2 )$$

where copper trace area and fiber (scaffold) area were calculated from image pixel counts and converted to mm² and total area is equal to 3×1 cm scaffold area (300 mm²) for all patterned designs.

Residual open area (%), a parameter allowing quantitation of macroscopic porosity of the fiber constructs generated was calculated as:

$$Residual Open Area ( \% ) = 100 - fiber target coverage \qquad ( 3 )$$

where fiber target coverage was calculated from Eq. 2.

To investigate the efficacy of each pattern as a target, and the accuracy of fiber deposition, i.e. how much coverage was achieved (full, less or excessive), fiber excess was determined as the total area of PLGA fiber adhesion which exceeded the bounds of the copper trace patterned area and was calculated as follows:

$$Fiber Excess = fiber ( scaffold ) area - copper trace area \qquad ( 4 )$$

where fiber (scaffold) area and copper trace area were calculated from image pixel counts and converted to mm² as previously mentioned for Eq. 1. While fiber excess is a characteristic parameter of the patterned collectors, in order to compare this parameter across differing designs it was normalized as follows:

$$Normalized Fiber Excess ( \% ) = ( fiber excess \div copper trace area ) \times 100 \qquad ( 5 )$$

where fiber excess was calculated from Eq. 4.

Grammage, a common variable utilized to determine densities of porous fibrous constructs (e.g. paper), was also calculated. Grammage is area density, and was calculated for the constructs as fiber grammage (μg/mm²) as follows:

$$Fiber Grammage = fiber scaffold mass \div fiber ( scaffold ) area \qquad ( 6 )$$

where fiber scaffold mass was measured utilizing an analytical balance and fiber (scaffold) area was measured as described in Eq. 1.

### Scaffold Mechanical Characterization

Mechanical characterization of scaffolds was determined using a Pyris Diamond Dynamic Mechanical Analyzer (DMA) (Perkin Elmer Waltham, MA, USA). Uniaxial tensile analysis was performed on scaffolds while stretched at 50 μm/minute to the point of failure. Elastic modulus and tensile strength were extrapolated from the stress versus strain data of each scaffold. Tensile strength was identified as the highest stress measured from the polymer construct during strain. All samples were strained to the point of failure. Elastic modulus was calculated according to Eq. 7, where F is force exerted on the polymer sample, A is the sample cross-sectional area, L₀ is the initial sample length, and ΔL is the change in sample length after straining:

$$E = Stress / Strain = ( F / A ) / ( Δ L / {L}_{0} ) \qquad ( 7 )$$

### Attenuated Total Reflectance-Fourier Transform Infrared (ATR-FTIR) Spectroscopy

The chemical composition of the electrospun PLGA fibers of each design were determined via Attenuated Total Reflectance-Fourier Transform Infrared (ATR-FTIR) Spectroscopy utilizing a Nicolet Avatar 360 FTIR spectrometer (Varian Inc., CA) equipped with a DTGS detector and Harrick MNP-Pro (Pleasantville, NY, USA) and attenuated total reflectance (ATR). Similar spectra were obtained of raw (pre-processing) PLGA and of spin coat PLGA as controls. Each spectrum was collected for 32 scans at a spectral resolution 2 cm⁻¹ over a wavenumber range of 4000-400 cm⁻¹. A background spectrum was carried out under the same experimental conditions and was subtracted from each sample spectrum. Spectral data were acquired with EZ-OMnic software.

### Contact Angle Determination

Static sessile drop angle of deionized water (DIW) on the surface of the fiber scaffolds was measured using an Optical System Cam 100 Optical Contact Angle Meter (DataPhysics Instruments USA Corp. 4424 Taggart Creek Road, Suite 102, Charlotte, NC 28208, USA). The hydrophobicity of differing fiber scaffolds was determined as follows: 10 μL of DIW was placed on top of each scaffold design. Three temporal images were taken on intervals of 1 sec. Computational analysis was used to calculate the contact angle of the obtained images. Test procedures were performed in triplicate per each fiber scaffold (n=3).

### Cell Culture

Primary human umbilical artery smooth muscle cells (HUASMCs) were cultured in smooth muscle cell growth medium (PromoCell GmbH, Heidelberg, Germany) supplemented with 1% (v/v) antibiotic-antimycotic (Life Technologies, Carlsbad, CA, USA), 5% (v/v) fetal bovine serum (Life Technologies, Carlsbad, CA, USA), 0.5 ng/mL EGF, 2 ng/mL bFGF, and 5 μg/mL insulin. All cells were incubated at 37° C., 5% CO₂ and grown to 70% confluency before use in experiments. Cells between passages 3 and 6 were employed for adhesion studies.

### Cell Adhesion and Retention

Prior to cell experiments, scaffolds were dry-incubated at 55° C. for 3 hours followed by a 15-min UV light exposure for sterilization. Scaffolds were cut into squares (1×1 cm) and pre-soaked in media for 10 minutes before cell seeding. HUASMCs (25,000 cells/mL) were added to each scaffold and incubated in a humidified incubator at 37° C. and 5% CO₂ for 24 hours. At 24 hrs, seeded scaffolds were washed in phosphate buffered saline (PBS)×3 to remove non-adherent cells. Scaffolds were then fixed in 4% (v/v in PBS) paraformaldehyde solution, followed by blocking buffer rinse (3% BSA v/v in PBS) and permeabilization (0.5% Triton v/v in PBS). Fluoroshield with DAPI (20 μL) was added to each scaffold which was then mounted on a microscope slide for imaging. Each scaffold was viewed and studied in five random regions, and three images were captured per region at 10× using an inverted fluorescent microscope (Axiovert 135, Carl Zeiss Microscopy LLC). Cells/microscope field were determined from resultant images using ImageJ™ using the cell counter plug-in.

### Statistical Analysis

All experiments were performed in triplicate, at a minimum. A two-tailed Welch's t-test was used to compare between means of each scaffold characterization, with a p-value <0.05 indicating significant differences. Correlation analysis between cell adhesion and scaffold parameters were performed using Pearson's correlation coefficient (R2).

### Results

### Physical Characterization of Patterned Scaffolds

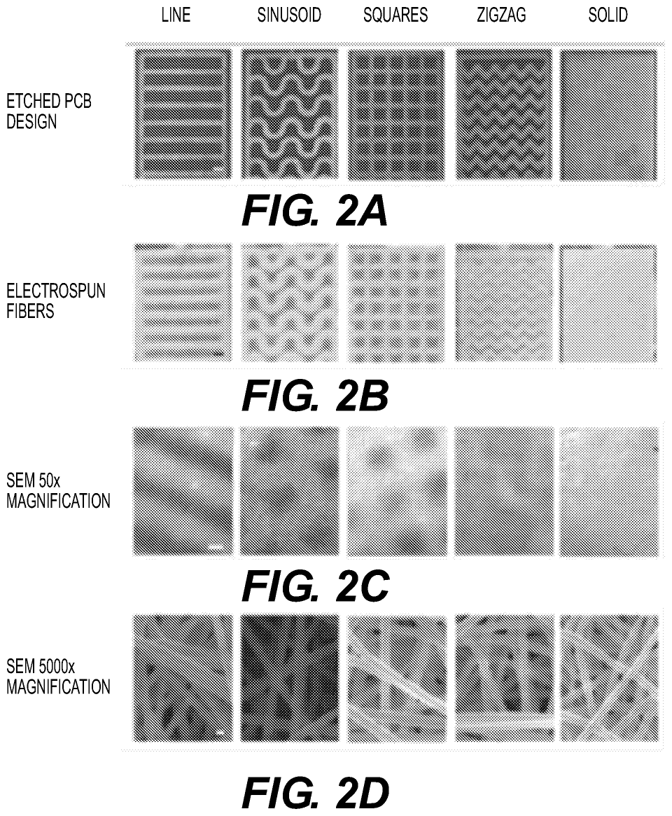

For the patterned collector designs tested, all were successfully transposed and etched onto copper-clad circuit board laminate as targets (FIG. 2A). Similarly, all trace designs generated were successful in serving as conductive targets, all accumulating PLGA fibers upon polymer jetting via electrospinning (FIG. 2B). No gapping or uneven distribution of fibers occurs macroscopically, or microscopically in terms of density, i.e. grammage. The efficacy of employing designated pattern electrospinning conductive targets was examined, with specific exposed electro-conductive trace patterns, as a means of generating defined, reproducible fiber constructs with a range of complexity. At the macroscopic level, each unique design was effective in generating a corresponding, distinguishable, electrospun polymer construct (FIG. 2B). Despite design complexity differences, and a range of target copper trace areas, i.e. ranging from 223.2 mm² (74% of the target exposed) to 132.3 mm² (44% of the target exposed), PLGA fibers remained largely confined to the traces, with the non-conductive inter-trace areas remaining polymer free.

As to individual fiber dimensions, variable, such as polymer concentration, solvent, molecular weight, applied voltage, distance to target, all remained constant in this study (i.e. use of a uniform electrospinning protocol) (Mo, et al., Biomaterials 2004, 25, 1883-1890; Yang, et al., Journal of Applied Physics 2008, 103, 104307). SEM of all fiber constructs generated here all revealed similar morphology at the level of resolution achievable by electron microscopic imaging. Despite differences in copper trace patterns, fiber diameter generated during electrospinning remained within a narrow range, varying from 4.1 μm to 3.4 μm, with a decline to 3.2 μm for a solid collector (Table 1). The differences observed in fiber diameter for each trace pattern were statistically significant. The mean fiber diameter for all designs tested was 3.6 μm.

Noticeably, differing patterns of collector trace designs differed in their degree of coverage by electrospun fibers. An inverse relationship was revealed between fiber excess and the degree of total target area exposed as usable copper target traces, i.e. the copper trace area (%). As exposed copper trace area increased from 44.1% to 74.4%, fiber excess decreased from 85.5+7.6 mm² to 52.2+11.9 mm² (p<0.05). Similarly, normalized fiber excess decreased from 64.6% to 23.4%. Having less exposed copper trace per overall target area (300 mm²) resulted in more generous coverage of traces, resulting in more robust deposition of fibers on traces, with an apparent spill-over of polymer bundles beyond trace boundary edges, (see e.g. FIGS. 2A and 2B for lines and sinusoid designs).

Differing target designs also impacted the area density or fiber grammage of fibers deposited, ranging from 24.9 μg/mm² for the line target pattern to 17.2 μg/mm² for a solid target (Table 1). Relatedly, fiber excess, with polymer bundle spill over, was also linearly related to fiber grammage. Fiber grammage was found to be directly related to fiber excess, ranging from 24.9 μg/mm² with a normalized excess of 64.6% (line design), to 17.6 μg/mm² with a normalized excess of 23.4% (zigzag design) (Table 1). Similar to fiber excess, fiber grammage was noted to be inversely related to copper trace area. As exposed target copper trace area decreased, the grammage or packing density of the fibers increased for a given design. Statistically significant differences in fiber grammage were found between line and zigzag target design (p<0.05) as well as between line and solid (p<0.05).

The results show that the different patterns on the targets controlled the subsequent electrospun fiber deposition, construct bulk configuration and scaffold macroscopic properties. For all designs examined, effective deposition of fibers was reproducibly detected, showing its efficacy as a viable processing methodology to create complex, shape-dictated constructs. Over a range of exposed traces ranging from 72.6% to 100% conductive surface, in no case did uneven distribution of fibers occur macroscopically, or microscopically in terms of density, i.e. grammage.

| | | Copper | Fiber | | | | | Normalized | Fiber | | |
|---|---|---|---|---|---|---|---|---|---|---|---|
| | Total | Trace | (scaffold) | Fiber | | Fiber | Fiber | Copper | Target | Normalized | Open |
| | Area | Area | Area | Diameter | Weight | Grammage | Excess | Trace Area | Coverage | Fiber | Area |
| | (mm²) | (mm²) | (mm²) | (μm) | (mg) | (μm/mm²) | (mm²) | (%) | (%) | Excess (%) | (%) |
| Lines | 300 | 132.3 | 217.8 ± 7.6 | 4.0 ± 0.0 | 5.4 ± 1.4 | 24.9 ± 0.8 | 85.5 ± 7.6 | 44.1 | 72.6 ± 1.3 | 64.6 ± 5.8 | 27.4 ± 2.5 |
| Sinusoids | 300 | 163.8 | 244.2 ± 12.1 | 3.4 ± 0.1 | 4.8 ± 1.4 | 19.6 ± 0.9 | 80.5 ± 12.7 | 54.6 | 81.4 ± 2.1 | 49.2 ± 7.8 | 18.6 ± 4.2 |
| Squares | 300 | 192.0 | 253 ± 19.9 | 3.6 ± 0.1 | 4.9 ± 2.1 | 19.6 ± 1.3 | 61.2 ± 19.9 | 64.0 | 84.4 ± 3.3 | 31.9 ± 10.3 | 15.6 ± 6.6 |
| Zigzags | 300 | 223.2 | 275.4 ± 11.9 | 4.1 ± 0.0 | 4.8 ± 1.5 | 17.6 ± 0.7 | 52.2 ± 11.9 | 74.4 | 91.8 ± 2.0 | 23.4 ± 5.3 | 8.2 ± 4.0 |
| Solid | 300 | 300 | 300 ± 0.0 | 3.2 ± 0.0 | 5.2 ± 1.9 | 17.2 ± 0.0 | 0.0 ± 0.0 | 100 | 100 ± 0.0 | 0.0 ± 0.0 | 0.0 ± 0.0 |

Values are mean ± standard deviation (n + 4). Total Area is the area (mm²) of the collector, including both conductive and non-conductive areas. Copper Trace Area is the area (mm²) of exposed copper trace for each design. Fiber (scaffold) Area is actual fiber coverage area (mm²) for each design. Fiber Diameter is the diameter (μm) measured of the fibers from SEM images. Fiber Grammage is “area density” of the scaffold, calculated as mass per Fiber (scaffold) Area of the scaffold. Normalized Copper Trace Area is the percentage of exposed copper of Total Area (300 mm²) of each collector. Fiber Target Coverage is the percent of Total Area (300 mm²) covered by electrospun fibers. Fiber Excess is the “spillover” area of the electrospun fibers which exceed the bounds of the copper trace. Normalized Fiber Excess is the amount of fiber excess in relation to the Copper Trace Area, an index of accuracy of the fibers distributing onto the collector. Residual Open Area is the percent of full target (300 mm²) not covered by electrospun scaffold, the inverse of Fiber Target Coverage.

### Mechanical Characterization of Patterned Scaffolds

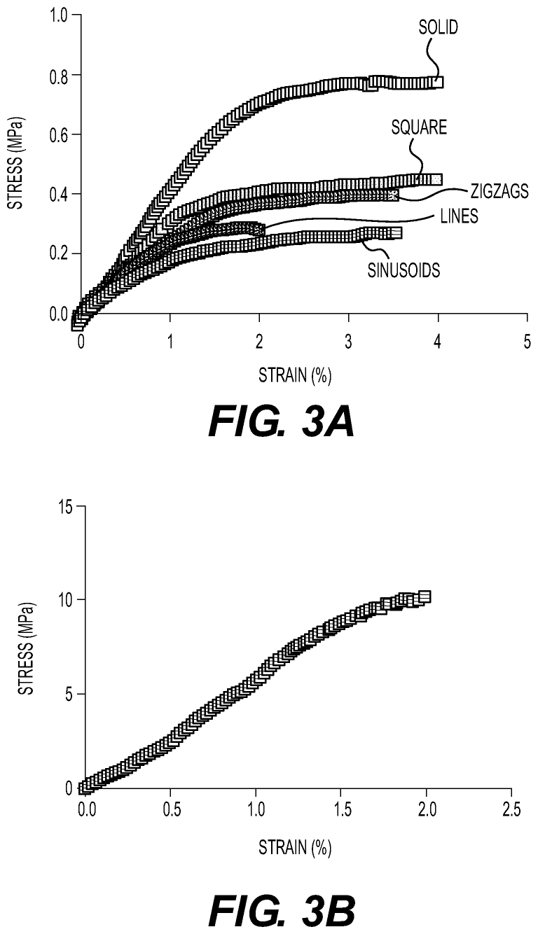

To define the bulk mechanical properties of the patterned scaffolds, dynamic mechanical analysis was performed on each design (3×1 cm). FIG. 3A reveals the stress-strain relationship with uniaxial tensile testing of each PLGA scaffold design. Spin-coat PLGA was also measured as comparative control (FIG. 3B). Young's modulus and tensile yield point of each scaffold design are listed in Table 2. Electrospun scaffold stiffness ranged from 0.3±0.2 MPa (line and sinusoid designs) to 0.6±0.4 MPa (solid design). As to tensile strength, the average yield point between scaffolds trended similarly to that of Young's Modulus, ranging between 0.4±0.4 MPa (lines) to 0.9±0.8 MPa (solid), though no statistical difference was noted for these parameters for the scaffold designs tested. Notably, the spin-coat scaffold had significantly higher stiffness and yield stress than all electrospun scaffolds tested, exhibiting approximately 10-fold increase in stiffness (Young's modulus=9.7±2.3 MPa) and 15-fold greater yield point (15.1±2.5 MPa). No significant difference in mechanical properties were noted between designs tested.

TABLE 2
Young's Modulus and Yield Point of Electrospun
Scaffolds and spin coated film

| | Young's Modulus | Yield Point |
|---|---|---|
| | (MPa) | (MPa) |
| Lines | 0.3 ± 0.2 | 0.4 ± 0.4 |
| Sinusoids | 0.3 ± 0.2 | 0.4 ± 0.3 |
| Squares | 0.4 ± 0.2 | 0.5 ± 0.4 |
| Zigzags | 0.3 ± 0.2 | 0.5 ± 0.4 |
| Solid | 0.6 ± 0.4 | 0.9 ± 0.8 |
| Spin Coat | 9.8 ± 5.6 | 15.1 ± 6.1 |

Values represent ± standard error (n = 6)

### Chemical Characterization of Scaffolds

FTIR was performed on all 5 electrospun target designs, i.e. lines, sinusoids, squares, zigzags, and solid. The spectra all show C—O peaks between 1000 and ˜1400 cm⁻¹, C═O peaks between 1700 and 1800 cm⁻¹, C—H peaks between 2800 and 3100 cm⁻¹, and O—H peaks between 3700 and 3900 cm⁻¹ (data not shown). No differences were detected between spectra generated from PLGA fibers scaffold designs tested. Similarly, no difference in spectra were noted between basal pre-processed PLGA and any of the post-spun PLGA, derived from the various scaffold designs. The results show that differing constructs had identical chemical composition and similar bulk mechanical properties.

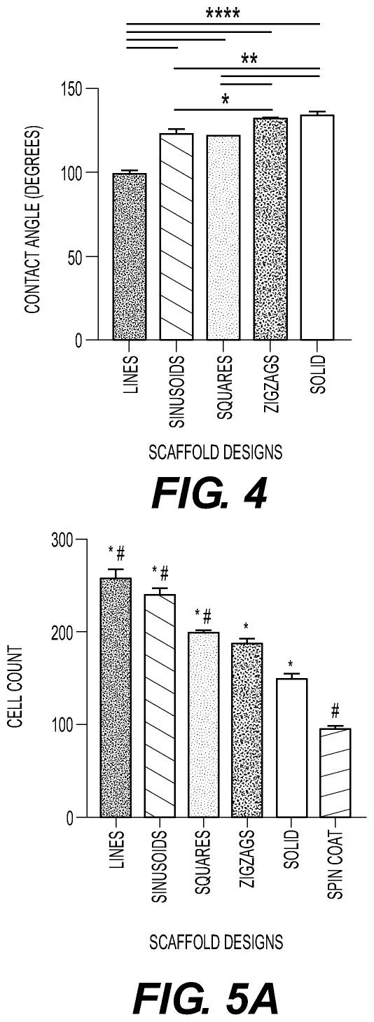

Wettability of the fiber scaffolds generated via differing targets were also assessed via measurement of contact angle (FIG. 4). Contact angle ranged from 98° with the line design, to 134° for the solid target design, a change of 36% relative to the line design. Contact angle was noted to vary inversely with fiber excess and with fiber grammage.

All fiber constructs generated utilized identical electrospinning conditions, i.e. polymer concentration, solvent, molecular weight, applied voltage, distance to target. Despite generating a range of shapes with differing fiber grammage, no difference in chemical composition was noted (Khalil, et al., International Journal of Electrochemical science 2013, 8, 3483-2493; Esmaili, et al., Journal of Biomedical Materials Research Part A 2017, 106, 285-292). What did differ between scaffold designs was surface free energy and overall hydrophobicity.

### Cell Adhesion and Retention of Scaffolds

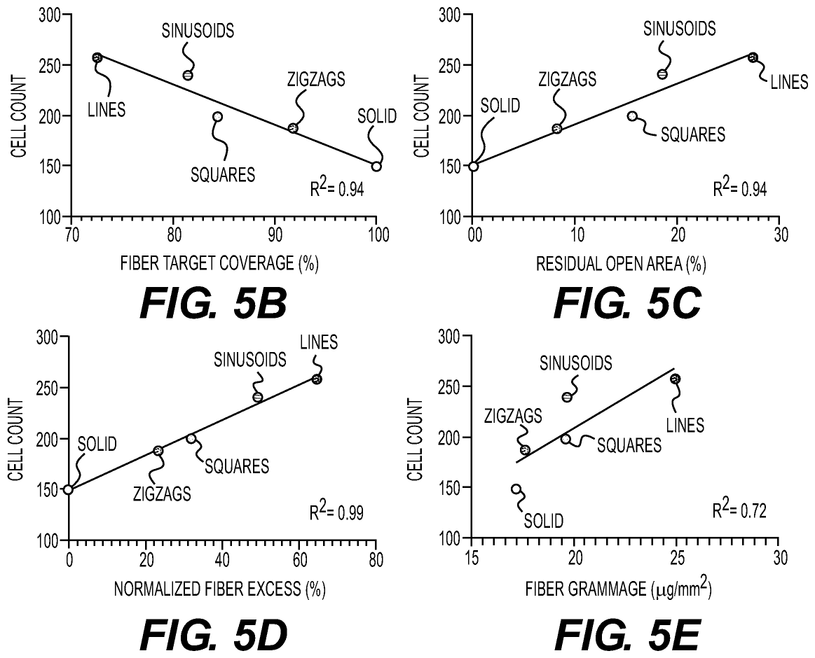

The impact of electrospun scaffold design on cell adhesion and retention at 24 hrs was determined via measurement of the number of vascular smooth muscle cells (VSMC) per microscope field detected in random regions of each construct post-incubation and washing. Of all scaffold designs tested, the lines design exhibited the highest adhesion and retention, followed by sinusoids and squares, which were all significantly greater than the solid electrospun sample (p<0.01; FIG. 5A). Cell counts ranged from 257.9 VSMC/field for the line design, to 149.8 VSMC/field for the solid target at 24 hours, a decrease of 42% relative to the line design. Cell adhesion was significantly lower with the spin-coat PLGA than all electrospun scaffolds tested (p<0.01).

As to the influence of bulk scaffold construct properties on cell adhesion and retention, there was an inverse relationship between cell adhesion and fiber target coverage (%). As overall target surface area coverage increased from 72.6% with the line design to 100% for the solid design, cell adhesion and retention decreased from 257.9 VSMC/field for the line design, to 149.8 VSMC/field at 24 hours, (R2=0.94; FIG. 5B).

As to macroporosity, there was a direct correlation between cell adhesion and retention at 24 hrs and scaffold residual open area (%). Cell adhesion varied from 149.8 VSMC/field for the solid design, to 257.9 VSMC/field for the line design, (R2=0.94; FIG. 5C).

As to the impact of bulk fiber density, there was a direct correlation between cell adhesion and retention at 24 hrs and normalized fiber excess (R2=0.99; FIG. 5D). At 0% fiber excess, as exists in the continuous solid target design, baseline cell adhesion of 149.8 VSMC/field was detected. As fiber excess increased to a maximum of 64.6% for the line design, cell adhesion and retention increased to 257.9 VSMC/field, an increase of 172% relative to the solid design. Similarly, this construct density to cell adhesion and retention relationship was also noted for fiber grammage (R2=0.72; FIG. 5E). As bulk construct density increased from a minimum of 17.2 μg/mm² with the solid construct, to a maximum of 24.9 μg/mm² for the line design, cell adhesion increased correspondingly.

The results show differing pattern constructs having distinct physical characteristics impact cell adhesion and retention. Specifically, macroscopic spacing between bulk fiber bundles, fiber bundle density and construct surface tension all influenced cell adhesion, retention and growth at 24 hours. Overall, the results demonstrate the ability to tune macro-scale fiber construct scaffold design, via manipulation of electrospinning target patterns, which ultimately influences micro-scale cell-material interactions.

Alteration of conductive electrospinning trace patterns is shown to be an effective means of directing and manipulating the macroscopic fabrication of electrospun fiber constructs. While utilizing similar electrical jetting parameters and a common polymeric solution a range of fiber target coverage can be achieved despite having an underlying common overall target area. Varying trace conductive area and spacing is an effective means of altering macroscopic fiber deposition, effectively modifying macroporosity. Trace spacing, via electrical field manipulation, alters fiber deposition density. All of these manipulations, while not impacting chemical and mechanical properties of constructs, directly alter cell adhesion and retention with greater macroporosity, fiber excess and fiber grammage directly impacting cell adhesion and retention, demonstrating their value as manipulable variables for construct fabrication for varying tissue engineering purposes.

1-5. (canceled)

6. A method of generating a fiber construct using an electrospinning system,
wherein the electrospinning system comprises an ejection device and a collector, wherein the collector comprises a collection surface comprising
a conductive trace or a plurality of conductive traces; and
a non-conductive region or a plurality of non-conductive regions,
wherein the conductive trace or the plurality of conductive traces form(s) a pattern on the collection surface,
wherein the collector has a conductive trace area from about 5% to about 99% of the total of the conductive trace area and a non-conductive area, optionally from about 20% to about 80% of the total of the conductive trace area and non-conductive area,
wherein the ejection device comprises a reservoir and an ejector, and wherein the reservoir comprises a polymer solution contained therein;
the method comprising
(ia) selecting the collector, wherein the collector has a collection surface comprising a pattern sufficient to generate the fiber construct having a grammage that is at least 5% higher than a grammage of a fiber construct formed on an un-patterned collector under the same conditions; and
(i) applying an electrostatic charge to the ejection device or a component thereof,
wherein following step (i), the polymer solution is extruded out of the ejection device via the ejector, wherein the polymer solution is directed to the collection surface and forms the fiber construct on the collection surface of the collector.

7. A method of generating a fiber construct using an electrospinning system,
wherein the electrospinning system comprises an ejection device and a collector, wherein the collector comprises a collection surface comprising
a conductive trace or a plurality of conductive traces; and
a non-conductive region or a plurality of non-conductive regions,
wherein the conductive trace or the plurality of conductive traces form(s) a pattern on the collection surface,
wherein the collector has a conductive trace area from about 5% to about 99% of the total of the conductive trace area and a non-conductive area, optionally from about 20% to about 80% of the total of the conductive trace area and non-conductive area,
wherein the ejection device comprises a reservoir and an ejector, and wherein the reservoir comprises a polymer solution contained therein;
the method comprising
(i) applying an electrostatic charge to the ejection device or a component thereof,
wherein following step (i), the polymer solution is extruded out of the ejection device via the ejector, wherein the polymer solution is directed to the collection surface, and forms the fiber construct on the collection surface of the collector,
wherein the formed fiber construct has a grammage that is at least 5% higher than a grammage of a fiber construct formed on an un-patterned collector under the same conditions.

8. The method of claim 6, further comprising loading the polymer solution into the reservoir of the ejection device prior to step (i), wherein the polymer solution comprises one or more polymers and a solvent, and optionally one or more active agent and/or one or more additive.

9. The method of claim 6, wherein the system further comprises a power supply, a pressurization component, a temperature control component, a position control component, one or more sensors, a controller, or an output device, or a combination thereof.

10. The method of claim 6, wherein the conductive trace or each conductive trace of the plurality of conductive traces is arranged such that during step (i), undisturbed electric field lines or blurred electric field lines align around the conductive trace or each conductive trace of the plurality of conductive traces.

11. The method of claim 8, wherein the polymer is a natural polymer or a synthetic polymer, or a combination thereof, and wherein the molecular weight of the natural polymer or synthetic polymer is in a range from about 1×10³ g/mol to about 500×10³ g/mol.

12. The method of claim 8, wherein the total concentration of the one or more polymer(s) is in a range from about 1% (w/v) to 90% (w/v) of the polymer solution.

13. The method of claim 8, wherein the polymer solution comprises two or more polymers, and wherein each polymer is present at a concentration in a range from about 0.1% (w/v) to 89.9% (w/v) of the polymer solution.

14. The method of claim 6, wherein the polymer solution is extruded from the ejection system at an extrusion rate in a range from about 0.01 mL/h to about 50 mL/h.

15. The method of claim 6, further comprising repeating steps (ia) and (i) or repeating step (i) at least one time to generate a fiber construct having at least two layers.

16. The method of claim 8, further comprising mixing the polymer with the solvent to form the polymer solution and/or assembling the system prior to step (i).

17. The method of claim 6, further comprising (ii) post-processing the fiber construct subsequent to step (i),
wherein step (ii) comprises triggering cross-linking in the fiber construct, shaping the fiber construct, cutting the fiber construct, heating the fiber construct, depositing a coating material on the surface of the fiber construct, and/or adding one or more active agent(s) and/or one or more additive(s) onto/into the fiber construct.

18. The method of claim 6, further comprising loading an active agent solution or suspension in a second reservoir prior to step (ia) or subsequent to step (ia) and prior to step (i).

19. The method of claim 8, wherein at least one of the one or more active agent(s) is associated with a carrier.

20. The method of claim 6, wherein the pattern on the collector comprises lines, checkerboard, squares, spirals, helices, zigzags, sinusoidal curves, non-sinusoidal wave forms, or circles, or a combination thereof.

21-58. (canceled)

59. The method of claim 7, further comprising loading the polymer solution into the reservoir of the ejection device prior to step (i), wherein the polymer solution comprises one or more polymers and a solvent, and optionally one or more active agent and/or one or more additive.

60. The method of claim 7, wherein the conductive trace or each conductive trace of the plurality of conductive traces is arranged such that during step (i) undisturbed electric field lines or blurred electric field lines align around the conductive trace or each conductive trace of the plurality of conductive traces.

61. The method of claim 59, wherein the polymer is a natural polymer or a synthetic polymer, or a combination thereof, and wherein the molecular weight of the natural polymer or synthetic polymer is in a range from about 1×10³ g/mol to about 500×10³ g/mol.

62. The method of claim 7, further comprising (ii) post-processing the fiber construct subsequent to step (i),
wherein step (ii) comprises triggering cross-linking in the fiber construct, shaping the fiber construct, cutting the fiber construct, heating the fiber construct, depositing a coating material on the surface of the fiber construct, and/or adding one or more active agent(s) and/or one or more additive(s) onto/into the fiber construct.

63. The method of claim 7, wherein the pattern on the collector comprises lines, checkerboard, squares, spirals, helices, zigzags, sinusoidal curves, non-sinusoidal wave forms, or circles, or a combination thereof.
